(19) United States
(12) Patent Application Publication
(10) Pub. No.: US 2006/0160814 A1
(43) Pub. Date: Jul. 20, 2006

(54) Phosphodiesterase 10 inhibitors

(76) Inventors: Mark Phillip Arrington, Westwood, NJ (US); Ruiping Liu, Huntington, NY (US); Richard D. Conticello, Ossining, NY (US); Carla Marcia Gauss, White Plains, NY (US); Allen Hopper, Glen Rock, NJ (US); Truc Minh Nguyen, New York, NY (US); Ashok Tehim, Ridgewood, NJ (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/217,664

(22) Filed: Sep. 2, 2005

Related U.S. Application Data
(60) Provisional application No. 60/606,895, filed on Sep. 3, 2004.

(51) Int. Cl. A61K 31/502; C07D 403/02

(52) U.S. Cl. 514/248; 544/235

(57) ABSTRACT

The present disclosure relates generally to the field of phosphodiesterase 10 (PDE10) enzyme inhibition by cinnoline compounds of Formulas I and II: 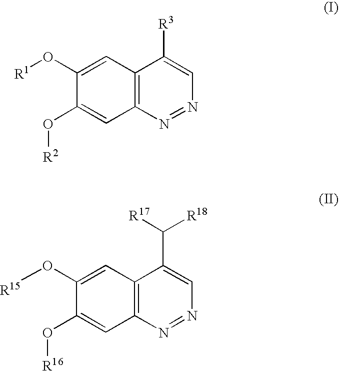
wherein R¹—R³ and R¹⁵—R¹⁸ are as defined herein. The disclosure also relates to methods of preparing such compounds, compositions containing such compounds, and methods of use thereof.

This application claims the benefit of U.S. application Ser. No. 60/606,895, filed Sep. 3, 2004, the entire disclosure of which is hereby incorporated by reference in its entirety.

### FIELD OF THE INVENTION

The present invention relates generally to the field of phosphodiesterase 10 (PDE10) enzyme inhibition. More specifically, this invention relates to selective PDE10 inhibition by novel compounds, e.g., cinnoline compounds, methods of preparing such compounds, compositions containing such compounds, and methods of use thereof.

### BACKGROUND OF THE INVENTION

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified so far, based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE 1 is stimulated by Ca²⁺/calmodulin. PDE 2 activity is stimulated by cGMP. PDE 3 is inhibited by cGMP. PDE 4 is cAMP specific and is specifically inhibited by rolipram. PDE 5 is cGMP-specific. PDE6 is expressed in retina. Less is known about the expression patterns and functional attributes of the higher number PDEs (7 through 11).

PDE10 sequences were first identified by using bioinformatics and sequence information from other PDE gene families (Fujishige et al., J. Biol. Chem. 274:18438-18445, 1999; Loughney, K. et al., Gene 234:109-117, 1999; Soderling, S. et al., Proc. Natl. Acad. Sci. USA 96:7071-7076, 1999). PDE10 is defined as a unique gene family based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least 3 alternative exons encoding the N and 2 for the C-termini. PDE10A1 is a 779 amino acid protein that hydrolyzes both cAMP and cGMP. The Km values for cAMP and cGMP are 0.05 and 3.0 micromolar, respectively. In addition to human variants, several variants with high homology have been isolated from both rat and mouse tissues and sequence banks.

PDE10 transcripts were initially detected in RNA from human testis and brain. Immunohistochemical analysis identified specific brain regions enriched in PDE10. The basal ganglia express the highest amounts of PDE10. Specifically, striatal neurons in the olfactory tubercle, caudate nucleus and nucleus accumbens are especially enriched in PDE10. Western blots did not reveal the expression of PDE10 in other brain tissues, although immunprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors may play an important role in the basal ganglia. PDE10A selective inhibitors could be used to raise levels of cAMP and/or cGMP within cells that express the PDE10 enzyme, especially neurons that comprise the basal ganglia. Selective PDE10A inhibition could lead to altered basal ganglia function and may be effective in treating a variety of neuropsychiatric conditions involving the basal ganglia.

### SUMMARY OF THE INVENTION

The present invention relates to novel compounds that inhibit, preferably selectively, PDE10 enzymes. In particular, the present invention relates to cinnoline compounds that are PDE10 inhibitors, compositions containing the same, methods of use thereof, and the synthesis thereof.

Still further, the present invention provides methods for synthesizing compounds with such activity and selectivity, as well as methods of and corresponding pharmaceutical compositions for treating a patient, e.g., mammals, including humans, in need of PDE inhibition. Treatment is preferably for a disease state that involves elevated intracellular PDE10 levels or decreased cAMP and/or cGMP levels, e.g., involving neurological or psychiatric syndromes, especially those states associated with psychoses, most especially schizophrenia or bipolar disorder, obsessive-compulsive disorder, and/or Parkinson's disease. In particular, such psychoses, obsessive-compulsive disorder, and/or Parkinson's disease are due at least in part to catabolism of intracellular cAMP and/or cGMP levels by PDE10 enzymes or where such an impaired condition can be improved by increasing cAMP and/or cGMP levels.

### DETAILED DESCRIPTION OF THE INVENTION

The present invention relates to inhibition of PDE10 enzymes, preferably selectively, by novel compounds, especially cinnoline compounds, methods of preparing such compounds, compositions containing such compounds, and methods of use thereof.

The present invention includes compounds of formulas I and II: 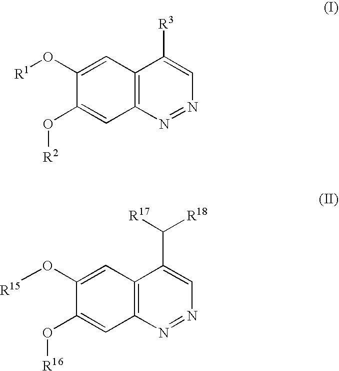
wherein
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from formulas (a)-(h): 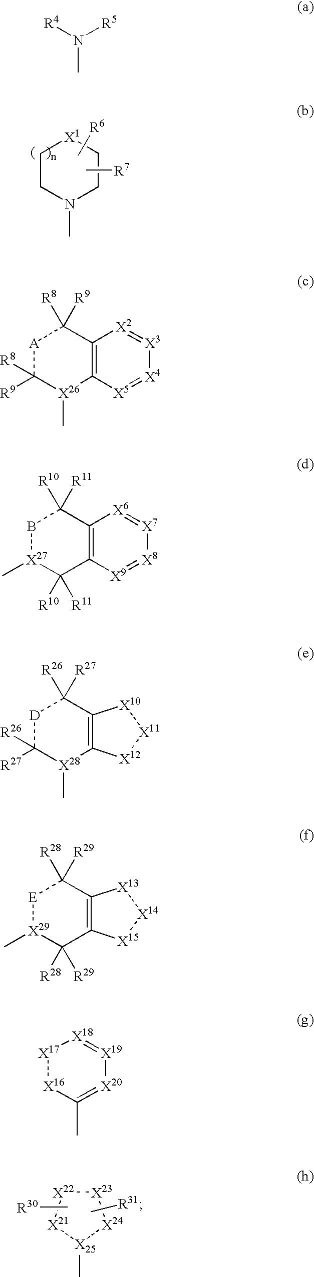
n is 0, 1, 2, or 3;
-A- is a single bond, a double bond, —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁸—, ═CR⁸—CR⁸═, —CR⁸R⁹—CR⁸R⁹—CR⁸R⁹—,═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸CR⁸—, —CR⁸═CR⁸═, or ═CR⁸—CR⁸R⁹—CR⁸═;
—B— is a single bond, a double bond, —CR¹⁰R¹¹—,═CR¹⁰—, —CR¹⁰═, —CR¹⁰R¹¹—CR¹⁰R¹¹—, ═CR¹⁰—CR¹⁰R¹¹—, —CR¹⁰R¹¹—CR¹⁰═, —CR¹⁰═CR¹⁰—, ═CR¹⁰—CR¹⁰═, —CR¹⁰R¹¹—CR¹⁰R¹¹—CR¹⁰R¹¹—, ═CR¹⁰—CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹—CR¹⁰R¹¹—CR¹⁰═, ═CR¹⁰—CR¹⁰═CR¹⁰—, —CR¹⁰═CR¹⁰—CR¹⁰═, or ═CR¹⁰—CR¹⁰R¹¹—CR¹⁰═,
   with the proviso that when X²⁷ is N, then —B— is not a double bond, ═CR¹⁰—, ═CR¹⁰—CR¹⁰R¹¹—, ═CR¹⁰—CR¹⁰═, ═CR¹⁰—CR¹R¹¹—CR¹⁰R¹¹—, ═CR¹⁰—CR¹⁰═CR¹⁰—, or ═CR¹⁰—CR¹⁰R¹¹—CR¹⁰═;
-D- is a single bond, a double bond, —CR²⁶R²⁷—, ═CR²⁶—, —CR²⁶═, —CR²⁶R^{27 —CR}²⁶R²⁷—, ═CR²⁶—CR²⁶R²⁷—,—CR²⁶R²⁷—CR²⁶═, —CR²⁶═CR²⁷—, ═CR²⁶—CR²⁶═, —CR²⁶R²⁷—CR²⁶R²⁷—CR²⁶R²⁷—, ═CR²⁶—CR²⁶R²⁷—CR²⁶R²⁷—, —CR²⁶═CR²⁶—CR²⁶R²⁷—, —CR²⁶R²⁷—CR²⁶═CR²⁶—, —CR²⁶R²⁷—CR²⁶R²⁷—CR²⁶═, ═CR²⁶—CR²⁶═CR²⁶—, —CR²⁶═CR²⁶—CR²⁶═, or ═CR²⁶—CR²⁶R²⁷—CR²⁶═;
-E- is a single bond, a double bond, —CR²⁸R²⁹—, ═CR²⁸—, —CR²⁸═, —CR²⁸R²⁹—CR²⁸R²⁹—, ═CR²⁸—CR²⁸R²⁹—, —CR²⁸ R²⁹—CR²⁸═, —CR²⁸═CR²⁹—, ═CR²⁸—CR²⁸═, —CR²⁸R²⁹—CR²⁸R²⁹—CR²⁸R²⁹—, ═CR²⁸—CR²⁸R²⁹—CR²⁸R²⁹—, —CR²⁸═CR²⁸—CR²⁸R²⁹—, —CR²⁸R²⁹—CR²⁸═CR²⁸—, —CR²⁸═R²⁹—CR²⁸═, ═CR²⁸—CR²⁹═CR²⁸—, —CR²⁸═CR²⁸—CR²⁸═, or ═CR²⁸—CR²⁸R²⁹—CR²⁸═,
   with the proviso that when X²⁹ is N, then -E- is not a double bond, ═CR²⁸—, ═CR²⁸—CR²⁸R²²⁹—, ═CR²⁸—CR²⁸═,═CR²⁸—CR²⁸R²⁹—CR²⁸R²⁹—, ═CR²⁸—CR²⁸═CR²⁸—, or ═CR²⁸—CR²⁸R²⁹—CR²⁸═;
the dotted lines in formula (e) independently represent a single bond or a double bond, wherein there is at least one double bond between X¹⁰ and X¹¹ or X¹¹ and X¹²;
the dotted lines in formula (f) independently represent a single bond or a double bond, wherein there is at least one double bond between X¹³ and X¹⁴ or X¹⁴ and X¹⁵;
the dotted line in formula (g) independently represents a single bond or a double bond (i.e., when there is a double bond between X¹⁶ and X¹⁷, formula (g) is aromatic);
the dotted lines in formula (h) independently represent a single bond or a double bond, with the proviso that when two double bonds are present, they are not adjacent to each other;
R⁴ and R⁵ are each independently
H,
straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹²,—CONHR¹²,—NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄ -alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently
H,
straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR², —CONHR², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹², or combinations thereof, or
R⁶ and R⁷ optionally form a cycloalkyl group, spiro or fused, having 3 to 8 carbon atoms, or
R⁶ and R⁷ together with the carbon to which they are attached form a C(═O) group;
X is O, S, NR¹³, CH₂, CHR⁶ or CR⁶R⁷;
X², X³, X⁴, X⁵, X⁶, X⁷, X⁸, and X⁹ are each independently N or CR¹⁴, and wherein two adjacent X²—X⁹ groups (e.g., X⁷ and X⁸) can each be CR¹⁴ in which the two R¹⁴ groups are together a methylenedioxy, ethylenedioxy, difluoromethylenedioxy, or tetrafluoroethylenedioxy group, to form a fused ring structure;
X¹⁰, X¹¹, X¹², X¹³, X¹⁴, and X¹⁵ are each independently S, O, N, NR¹⁴, C(R¹⁴)₂, or CR¹⁴ (e.g., X¹³ is S and X¹⁴ and X¹⁵ are CR¹⁴ (e.g., CH));
X¹⁶, X¹⁷, X¹⁸, X¹⁹, and X²⁰, are each independently N or CR¹⁴ (for example, CH) (e.g., (i) X¹⁶, X¹⁷, X¹⁸, and X²⁰ are CH, and X¹⁹ is CR¹⁴, (ii) X¹⁶, X¹⁷, X¹⁸ and X²⁰ are CH and X¹⁹ is N),
X¹⁶ and X¹⁷ can also each, independently, be NR¹⁴ or C(R¹⁴)₂, and
X¹⁸ and X¹⁹ or X¹⁹ and X²⁰ optionally form a fused aryl or heteroaryl, each of which may be substituted by one or more R¹⁴ groups;
X²¹, X²², X²³, and X²⁴ are each independently O, S, N, NR¹⁴, CR¹⁴, or C(R¹⁴)₂;
X²⁵ is N, C or CR¹⁴;
wherein at least two of X²¹, X²², X²³, X²⁴, and X²⁵ are each, independently, O, S, N, or NR¹⁴;
X²⁶ is N or CR⁸;
X²⁷ is C, N, or CR¹⁰;
X²⁸ is N or CR²⁶;
X²⁹ is C, N, or CR²⁸;
R⁸, R⁹, R¹⁰, R¹¹, R²⁶, R²⁷, R²⁸, and R²⁹ are, in each case, independently
   absent, H, or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof; or
   R⁸ and R⁹, R¹⁰ and R¹¹, R²⁶ and R²⁷, and/or R²⁸ and R²⁹ together optionally form a cycloalkyl group, spiro or fused, having 3 to 8 carbon atoms, or
   one or more of R⁸ and R⁹ and the carbon atom to which they are attached, or one or more of R¹⁰ and R¹¹ and the carbon atom to which they are attached, or one or more of R²⁶ and R²⁷ and the carbon atom to which they are attached, or one or more of R²⁸ and R²⁹ and the carbon atom to which they are attached, in each case form a C(═O) group;
R¹² is H or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof;
R¹³ H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   —CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR , —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   a heterocyclic group, which is saturated, partially saturated, or unsaturated, having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, (e.g., pyridinyl, thiazolyl, indolyl, thienyl, pyrimidinyl),
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof (e.g., benzyl),
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   aryloxy having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof (e.g., phenoxy),
   heteroaryloxy having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₇₋₁₆ arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   O-heterocyclic group, in which the heterocyclic group is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, (e.g., tetrahydrofuranyloxy);
   O-heterocyclicalkyl group, in which the heterocyclic group is nonaromatic, having 5 to 10 ring atoms in which at least 1 ring atom is a heteroatom (preferably 1 to 4 heteroatoms, preferably the heteroatoms are selected from N, S, and O), and the alkyl portion has 1 to 3 carbon atoms and the heterocyclic group is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, (e.g., morpholinylethoxy);
   or
   halogen (preferably F), hydroxy, C₁₋₄-alkoxy (e.g., OCH₃), C₁₋₄-alkyloxyC₁₋₄-alkoxy (e.g., methoxyethoxy (—OCH₂CH₂OCH₃)), C₄₋₁₂-cycloalkylalkyloxy (e.g., O-cyclopropylmethyl), C₁₋₄-alkyloxyC₇₋₁₆-arylalkyloxy (e.g., OCH₂CH₂OCH₂C₆H₅), halogenated C₁₋₄ alkoxy (e.g., OCHF₂, OCF₃), nitro, cyano, carboxy, amino, C₁₋₄ alkylamino, di-C₁₋₄ -alkylamino, C₁₋₄-hydroxyalkyl, C₂₋₄-hydroxyalkoxy (e.g., OCH₂CH₂OH), —COR¹², —COOR¹², —OCOR¹², C₁₋₄-alkylthio, C₁₋₄-alkylsulphinyl, C₁₋₄-alkylsulphonyl, —SO₂NHR¹² (e.g., SO₂NHCH₃), —SO₂NHR²⁵ (e.g., SO₂NH-cyclopropylmethyl), —SO₂NR¹⁹R²⁵ (e.g., SO₂N(CH₃)₂), —SO₂R³² (e.g., —SO₂-piperidine), —SO₂-pyrrolidine)), —NHSO₂R¹², —NR¹²COR¹², —CONHR¹² (e.g., CONH-alkyl, such as CONHCH₂CH₃, CONHCH₂CH(CH₃)₂, CONH-cycloalkyl, such as CONH-cyclopropyl), —CONR¹²R²⁵ (e.g., CON(CH₂CH₃)₂, C₁₋₄ alkyl-CONR¹²R²⁵, —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₇₋₁₆ arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is-unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
R¹⁹ is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
R²⁵ is H,
   alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
   cycloalkyl having 3 to 10, preferably 3 to 8 carbon atoms, which is unsubstituted or substituted one or more times with halogen, C₁₋₄-alkyl, C₁₋₄-alkoxy, oxo, or combinations thereof (e.g., cyclopropyl),
   cycloalkylalkyl having 4-12 carbon atoms which is unsubstituted or substituted one or more times with halogen, C₁₋₄-alkyl, C₁₋₄-alkoxy, oxo, or combinations thereof (e.g., cyclopropylmethyl),
   heterocyclic group which is saturated, partially saturated, or unsaturated, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof (e.g., pyrrolidinyl, piperidinyl); or
   heterocyclicalkyl group wherein the heterocyclic group has 5 to 10 ring atoms in which at least 1 ring atom is a heteroatom (preferably 1 to 4 heteroatoms, preferably the heteroatoms are selected from N, S, and O) and the alkyl portion has 1 to 4 carbon atoms, the heterocyclic group is unsubstituted or is substituted one or more times by halogen C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof (e.g., morpholinylethyl);
R³⁰ and R³¹ are, in each case, independently
   H or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof; or
   R³⁰ and R³¹ form a cycloalkyl group, spiro or fused, having 3 to 8 carbon atoms, or
   R³⁰ and R³¹ and the carbon atom to which they are attached form a C(═O) group;
R³² is a heterocyclic group which is saturated or partially saturated and has 5 to 10 ring atoms in which at least 1 ring atom is a heteroatom (preferably 1 to 4 heteroatoms, preferably the heteroatoms are selected from N, S, and O) and which is unsubstituted or substituted one or more times by halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof;

and pharmaceutically acceptable salts or solvates (e.g., hydrates) thereof, or solvates of pharmaceutically acceptable salts thereof;

with the proviso that said compound is not

4-(4-methoxyanilino)-6,7-dimethoxycinnoline,

4-(4-ethoxyanilino)-6,7-dimethoxycinnoline,

4-(4-methylanilino)-6,7-dimethoxycinnoline,

4-(3,4-dimethylanilino)-6,7-dimethoxycinnoline,

4-(2-chloroanilino)-6,7-dimethoxycinnoline,

4-(3-chloroanilino)-6,7-dimethoxycinnoline,

4-(4-chloroanilino)-6,7-dimethoxycinnoline,

4-(3-bromoanilino)-6,7-dimethoxycinnoline,

4-(3-hydroxy-4-methylanilino)-6,7-dimethoxycinnoline,

4-(2-fluoro-5-hydroxy-4-methylanilino)-6,7-dimethoxycinnoline,

4-(4-chloro-2-fluoro-5-hydroxyanilino)-6,7-dimethoxycinnoline,

6,7-dimethoxy-4-(1-piperazinyl)cinnoline,

4-amino-6,7-dimethoxycinnoline,

4-anilino-6,7-dimethoxycinnoline,

6,7-dimethoxy-α-1-naphthyl-4-cinnoline-acetonitrile,

4-(4-aminobenzyl)-6,7-dimethoxy-cinnoline,

6,7-dimethoxy-α-(3-methoxyphenyl)-4-cinnoline-acetonitrile,

α-[4,5-dihydro-4,4-dimethyl-1-(1-methylethyl)-1H-imidazol-2-yl]-6,7-dimethoxy-4-cinnolineacetonitrile,

α-(3,4-dimethoxyphenyl)-6,7-dimethoxy-4-cinnoline-acetamide,

6,7-dimethoxy-α-phenyl-4-cinnoline-acetonitrile,

α-(3,4-dimethoxyphenyl)-6,7-dimethoxy-4-cinnoline-acetonitrile,

6,7-dimethoxy-α-(4-iodophenyl)-4-cinnoline-acetonitrile,

6,7-dimethoxy-α-(4-bromophenyl)-4-cinnoline-acetonitrile,

6,7-dimethoxy-α-(4-chlorophenyl)-4-cinnoline-acetonitrile,

α-(3,4-dichlorophenyl)-6,7-dimethoxy-4-cinnoline-acetonitrile,

6,7-dimethoxy-α-(phenyl)-4-cinnoline-acetamide (also called α-(6,7-dimethoxy-4-cinnolyl)phenylacetamide),

α-(4-aminophenyl)-6,7-dimethoxy-4-cinnolinea-cetonitrile, or

4-benzyl-6,7-dimethoxycinnoline,

or a pharmaceutically acceptable salt thereof, or a solvate thereof, or a solvate of a pharmaceutically acceptable salt thereof.

In one aspect of the invention, R³ is of formula (a). In a further aspect of the invention, R³ is of formula (b).

According to another aspect, R³ is of formula (c) and (d). In a further aspect of the invention, R³ is of formula (c). In a further aspect of the invention, R³ is of formula (d).

According to another aspect, R³ is of formula (e) and (f). In a further aspect of the invention, R³ is of formula (e). In a further aspect of the invention, R³ is of formula (f).

According to another aspect, R³ is of formula (g). In a further aspect of the invention, R³ is of formula (h).

According to a further aspect, the invention includes compounds selected from subgerenric formulas I (a) and II (a) which correspond to formulas I and II, respectively, but in which R¹—R³ and R¹⁵—R¹⁸ are defined as follows:
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from: 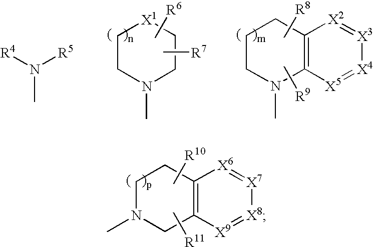
n is 0, 1, 2, or 3;
m is 0, 1, 2, or 3;
p is 0, 1, 2, or 3;
R⁴ and R⁵ are each independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹², or combinations thereof, or
   R⁶ and R⁷ optionally form a cycloalkyl group, spiro or fused, having 3 to 8 carbon atoms,
X¹ is O, S, NR¹³, CH₂, CHR⁶ or CR⁶R⁷;
X², X³, X⁴, X⁵, X⁶, X⁷, X⁸, and X⁹ are each independently N or CR¹⁴, and wherein two adjacent X²—X⁹ groups (e.g., X⁷ and X⁸) can together be a methylenedioxy, ethylenedioxy group, difluoromethylenedioxy, or tetrafluoromethylenedioxy, to form a fused ring structure;
R⁸ and R⁹ are in each case independently
   H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof, or
   R⁸ and R⁹ form a cycloalkyl group, spiro or fused, having 3 to 8 carbon atoms;
R¹⁰ and R¹¹ are in each case independently
   H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof, or
   R¹⁰ and R¹¹ form a cycloalkyl group, spiro or fused, having 3 to 8 carbon atoms;
R¹² is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
R¹³ H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   —CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄ -hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C¹⁻⁴-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C¹⁻⁴-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
R¹⁹ is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;

and pharmaceutically acceptable salts or solvates (e.g., hydrates) thereof, or solvates of pharmaceutically acceptable salts thereof;

with the proviso that said compound is not

4-(4-methoxyanilino)-6,7-dimethoxycinnoline,

4-(4-ethoxyanilino)-6,7-dimethoxycinnoline,

4-(4-methylanilino)-6,7-dimethoxycinnoline,

4-(3,4-dimethylanilino)-6,7-dimethoxycinnoline,

4-(3-chloroanilino)-6,7-dimethoxycinnoline,

4-(2-chloroanilino)-6,7-dimethoxycinnoline,

4-(4-chloroanilino)-6,7-dimethoxycinnoline,

4-(3-bromoanilino)-6,7-dimethoxycinnoline,

4-(3-hydroxy-4-methylanilino)-6,7-dimethoxycinnoline,

4-(2-fluoro-5-hydroxy-4-methylanilino)-6,7-dimethoxycinnoline,

4-(4-chloro-2-fluoro-5-hydroxyanilino)-6,7-dimethoxycinnoline,

6,7-dimethoxy-4-(1-piperazinyl)cinnoline,

4-amino-6,7-dimethoxycinnoline,

4-anilino-6,7-dimethoxycinnoline,

6,7-dimethoxy-α-1-naphthyl-4-cinnoline-acetonitrile,

4-(p-aminobenzyl)-6,7-dimethoxy-cinnoline,

6,7-dimethoxy-α-(m-methoxyphenyl)-4-cinnoline-acetonitrile,

α-[4,5-dihydro-4,4-dimethyl-1-(1-methylethyl)-1H-imidazol-2-yl]-6,7-dimethoxy-4-cinnolineacetonitrile,

α-(3,4-dimethoxyphenyl)-6,7-dimethoxy-4-cinnoline-acetamide,

6,7-dimethoxy-α-phenyl-4-cinnoline-acetonitrile,

α-(3,4-dimethoxyphenyl)-6,7-dimethoxy-4-cinnoline-acetonitrile,

6,7-dimethoxy-α-(p-iodophenyl)-4-cinnoline-acetonitrile,

6,7-dimethoxy-α-(p-bromophenyl)-4-cinnoline-acetonitrile,

α-(3,4-dichlorophenyl)-6,7-dimethoxy-4-cinnoline-acetonitrile,

6,7-dimethoxy-α-(phenyl)-4-cinnoline-acetamide (also called α-(6,7-dimethoxy-4-cinnolyl)phenylacetamide),

α-(4-chlorophenyl)-6,7-dimethoxy-4-cinnolineacetonitrile,

α-(4-aminophenyl)-6,7-dimethoxy-4-cinnolineacetonitrile, or

4-benzyl-6,7-dimethoxycinnoline,

or a pharmaceutically acceptable salt thereof, or a solvate thereof, or a solvate of a pharmaceutically acceptable salt thereof.

According to one aspect of the invention, the compounds are selected from those of formula I. In a further aspect of the invention, the compounds are selected from those of formula I(a).

According to a further aspect, the invention includes compounds of Formulas I or Ia wherein when n is 1 and X¹ is NH, R⁶ and R⁷ are not both H.

According to a further aspect, the invention includes compounds of Formulas I or Ia wherein when one of R⁴ or R⁵ is H, unsubstituted phenyl, or phenyl substituted by alkyl, hydroxyl and/or halogen, the other is not H.

According to a further aspect, the invention includes compounds of Formulas I or Ia wherein when n is 1 and X¹ is NH, R⁶ and R⁷ are not both H, and when one of R⁴ or R⁵ is H, unsubstituted phenyl, or phenyl substituted by alkyl, hydroxyl and/or halogen, the other is not H.

According to a further aspect, the invention includes compounds of Formulas I or Ia wherein when one of R⁴ and R⁵ is H or substituted or unsubstituted phenyl, the other is not H.

According to a further aspect, the invention includes compounds of Formulas I or Ia wherein when n is 1 and X¹ is NH, R⁶ and R⁷ are not both H, and when one of R⁴ and R⁵ is H or substituted or unsubstituted phenyl, the other is not H.

According to a further aspect, the invention includes compounds of Formulas I or Ia wherein —NR⁴R⁵ is not NH₂, NHCH₃, or substituted or unsubstituted anilino.

According to a further aspect, the invention includes compounds of Formulas I or Ia wherein —NR⁴R⁵ is not NH₂, unsubstituted monoalkylamino, or substituted or unsubstituted anilino.

According to a further aspect, the invention includes compounds of Formulas I or Ia wherein —NR⁴R⁵ is not NH₂, unsubstituted monoalkylamino, unsubstituted dialkylamino, or substituted or unsubstituted anilino.

According to one aspect of the invention, the compounds are selected from those of formula II. In a further aspect of the invention, the compounds are selected from those of formula II(a).

According to a further aspect, the invention includes compounds of Formulas II or II(a), wherein when R¹⁸ is cyano, then R¹⁷ is other than halo-substituted phenyl.

According to a further aspect, the invention includes compounds of Formulas II or II(a), wherein R¹⁸ is other than H.

According to a further aspect, the invention includes compounds of Formulas II or II(a) wherein R¹⁸ is not H, cyano, or —CONHR¹⁹.

According to a further aspect, the invention includes compounds of Formula III wherein 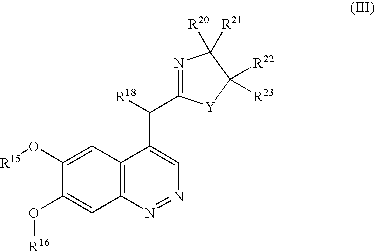
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
Y is NR²⁴, O or S;
R²⁰, R^{21,} R²², and R²³ are independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkyl amino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof, or
   cyano, carboxy, C₁₋₄-hydroxyalkyl, COR¹⁹, COOR¹⁹, CONHR¹⁹ or combinations thereof,
   wherein two of R²⁰, R²¹, R²², and R²³ together may optionally form a spiro or fused cycloalkyl group having 3 to 8 carbon atoms, and R²⁰ and R²¹ or R²² and R²³ together may optionally form an oxo group;
R²⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;

or a pharmaceutically acceptable salt or solvate thereof, or a solvate of a pharmaceutically acceptable salt thereof;

wherein said compound is not α-[4,5-dihydro-4,4-dimethyl-1-(1-methylethyl)-1H-imidazol-2-yl]-6,7-dimethoxy-4-cinnolineacetonitrile, or a pharmaceutically acceptable salt thereof, or a solvate thereof, or a solvate of a pharmaceutically acceptable salt thereof.

According to a further aspect, the invention includes compounds of Formula III, wherein if R²⁴ is isopropyl, then R²⁰ and R²¹ are not both methyl.

According to a further aspect, the invention includes compounds of Formula III, wherein if R²⁴ is isopropyl, then R²⁰ and R²¹ are not both alkyl.

According to a further aspect, the invention includes compounds of Formula III, wherein R¹⁸ is other than H.

According to a further aspect, the invention includes compounds of Formula III, wherein R¹⁸ is not H, cyano, or —CONHR¹⁹.

The compounds of the present invention are effective in inhibiting, or modulating the activity of PDE10 in animals, e.g., mammals, especially humans. These compounds exhibit activity, especially where such activity affects states associated with psychoses, especially schizophrenia or bipolar disorder, obsessive-compulsive disorder, and Parkinson's disease, including long term memory. These compounds will also be effective in treating diseases where decreased cAMP and/or cGMP levels are involved.

Assays for determining PDE10 inhibiting activity, selectivity of PDE10 inhibiting activity, and selectivity of inhibiting PDE4 isoenzymes are known within the art. See, e.g., U.S. Published Application No. 2004/0162293. See also, e.g., Example 15.

According to a method aspect, the invention includes administering to a patient a compound selected from formulas I and II: 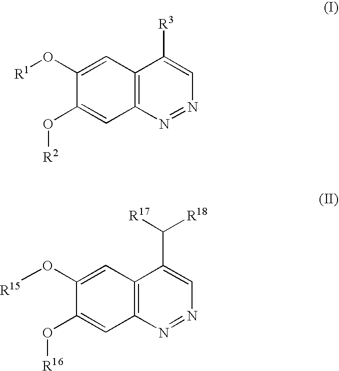
wherein
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from formulas (a)-(h): 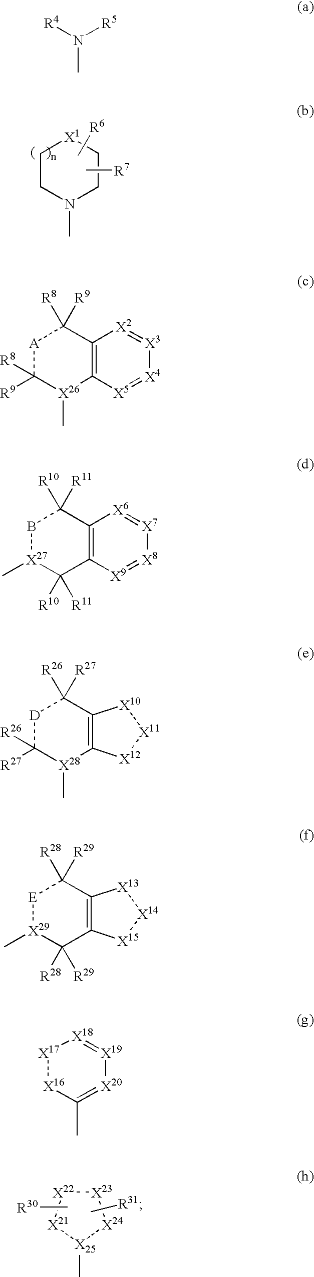
n is 0, 1, 2, or 3;
-A- is a single bond, a double bond, —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁸—, ═CR⁸—CR⁸═, —CR⁸R⁹—CR⁸R⁹—CR⁸R⁹—,═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, or ═CR⁸—CR⁸R⁹—CR⁸═;
—B— is a single bond, a double bond, —CR¹⁰R¹¹—, ═CR¹⁰—, —CR¹⁰═, —CR¹⁰R¹¹—CR¹⁰R¹¹—, ═CR¹⁰—CR¹⁰R¹¹—, —CR¹⁰R¹¹—CR¹⁰═, —CR¹⁰═CR¹⁰—, ═CR¹⁰—CR¹⁰—, —CR¹⁰R¹¹—CR¹⁰R¹¹—CR¹⁰R¹¹—, ═CR¹⁰—CR¹⁰R¹¹—CR¹⁰R¹¹—, —CR¹⁰-═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, ═CR¹⁰—CR¹⁰═CR¹⁰—, —CR¹⁰═CR¹⁰—CR¹⁰═, or ═CR¹⁰—CR¹⁰R¹¹—CR¹⁰═,
   with the proviso that when X²⁷ is N, then —B— is not a double bond, ═CR¹⁰—, ═CR¹⁰—CR¹⁰R¹¹—, ═CR¹⁰—CR¹⁰═, ═CR¹⁰—CR¹⁰R¹¹—CR¹⁰R¹¹—, ═CR¹⁰—CR¹⁰═CR¹⁰—, or ═CR¹⁰—CR¹⁰R¹¹—CR¹⁰═;
-D- is a single bond, a double bond, —CR²⁶R²⁷—, ═CR²⁶—, —CR²⁶═, —CR²⁶R²⁷—CR²⁶R²⁷—, ═CR²⁶—CR²⁶R²⁷—, —CR²⁶R²⁷—CR²⁶═, —CR²⁶═CR²⁷—,═CR²⁶—CR²⁶═, —CR²⁶R²⁷—CR²⁶R²⁷—CR²⁶R²⁷—, ═CR²⁶—CR²⁶R²⁷—CR²⁶R²⁷, —CR²⁶═CR²⁶—CR²⁶R²⁷—, —CR²⁶R²⁷—CR²⁶═CR²⁶—, —CR²⁶R²⁷—CR²⁶R²⁷—CR²⁶═, ═CR²⁶—CR²⁶═CR²⁶—, —CR²⁶═CR²⁶—CR²⁶═, or ═CR²⁶—CR²⁶R²⁷—CR²⁶═;
-E- is a single bond, a double bond, —CR²⁸R²⁹—, ═CR²⁸—, —CR²⁸═, —CR²⁸R²⁹—CR²⁸R²⁹—, ═CR²⁸—CR²⁸R²⁹, —CR²⁸R²⁹—CR²⁸═, —CR²⁸═CR²⁹—,═CR²⁸—CR²⁸═, —CR²⁸R²⁹—CR²⁸R²⁹—CR²⁸R²⁹—, ═CR²⁸—CR²⁸R²⁹—CR²⁸R²⁹—, —CR²⁸═CR²⁸—CR²⁸R²⁹—, —CR²⁸R²⁹—CR²⁸═CR²⁸—, —CR²⁸R²⁹—CR²⁸R²⁹—CR²⁸═, ═CR²⁸—CR²⁹═CR²⁸—, —CR²⁸═CR²⁸—CR²⁸═, or ═CR²⁸—CR²⁸R²⁹—CR²⁸═,
   with the proviso that when X²⁹ is N, then -E- is not a double bond, ═CR²⁸—, ═CR²⁸—CR²⁸R²²⁹—, ═CR²⁸—CR²⁸═, ═CR²⁸—CR²⁸—CR²⁸R²⁹—CR²⁸R²⁹—, ═CR²⁸—CR²⁸═CR²⁸—, or ═CR²⁸—CR²⁸R²⁹—CR²⁸═;
the dotted lines in formula (e) independently represent a single bond or a double bond, wherein there is at least one double bond between X¹⁰ and X¹¹ or X¹¹ and X¹²;
   the dotted lines in formula (f) independently represent a single bond or a double bond, wherein there is at least one double bond between X¹³ and X¹⁴ or X¹⁴ and X¹⁵;
   the dotted line in formula (g) independently represents a single bond or a double bond (i.e., when there is a double bond between X¹⁶ and X¹⁷, formula (g) is aromatic);
   the dotted lines in formula (h) independently represent a single bond or a double bond, with the proviso that when two double bonds are present, they are not adjacent to each other;
R⁴ and R⁵ are each independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹², or combinations thereof, or
   R⁶ and R⁷ optionally form a cycloalkyl group, spiro or fused, having 3 to 8 carbon atoms, or
   R⁶ and R⁷ together with the carbon to which they are attached form a C(═O) group;
X is O, S, NR¹³, CH₂, CHR⁶ or CR⁶R⁷;
X², X³, X⁴, X⁵, X⁶, X⁷, X⁸, and X⁹ are each independently N or CR¹⁴, and wherein two adjacent X²—X⁹ groups (e.g., X⁷ and X⁸) can each be CR¹⁴ in which the two R¹⁴ groups are together a methylenedioxy, ethylenedioxy, difluoromethylenedioxy, or tetrafluoroethylenedioxy group, to form a fused ring structure;
X¹⁰, X¹¹, X¹², X¹³, X¹⁴, and X¹⁵ are each independently S, O, N, NR¹⁴, C(R¹⁴)₂, or CR¹⁴ (e.g., X¹³ is S and X¹⁴ and X¹⁵ are CR¹⁴ (e.g., CH));
X¹⁶, X¹⁷, X¹⁸, X¹⁹, and X²⁰, are each independently N or CR¹⁴ (for example, CH) (e.g., (i) X¹⁶, X¹⁷, X¹⁸, and X²⁰ are CH, and X¹⁹ is CR¹⁴, (ii) X¹⁶, X¹⁷, X¹⁸ and X²⁰ are CH and X¹⁹ is N),
X¹⁶ and X¹⁷ can also each, independently, be NR¹⁴ or C(R¹⁴)₂, and
X¹⁸ and X¹⁹ or X¹⁹ and X²⁰ optionally form a fused aryl or heteroaryl, each of which may be substituted by one or more R¹⁴ groups;
X¹², X²², X²³, and X²⁴ are each independently O, S, N, NR¹⁴, CR¹⁴, or C(R¹⁴)₂;
X²⁵ is N, C or CR¹⁴;
   wherein at least two of X²¹, X²², X²³, X²⁴, and X²⁵ are each, independently, O, S, N, or NR¹⁴;
X²⁶ is N or CR⁸;
X²⁷ is C, N, or CR¹⁰;
X²⁸ is N or CR²⁶;
X²⁹ is C, N, or CR²⁸;
R⁸, R⁹, R¹⁰, R¹¹, R²⁶, R²⁷, R²⁸, and R²⁹ are, in each case, independently
   absent, H, or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof; or
   R⁸ and R⁹, R¹⁰ and R¹¹, R²⁶ and R²⁷, and/or R²⁸ and R²⁹ together optionally form a cycloalkyl group, spiro or fused, having 3 to 8 carbon atoms, or
   one or more of R⁸ and R⁹ and the carbon atom to which they are attached, or one or more of R¹⁰ and R¹¹ and the carbon atom to which they are attached, or one or more of R²⁶ and R²⁷ and the carbon atom to which they are attached, or one or more of R²⁸ and R²⁹ and the carbon atom to which they are attached, in each case form a C(═O) group;
R¹² is H or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof;
R¹³ H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄ alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   —CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   a heterocyclic group, which is saturated, partially saturated, or unsaturated, having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₁₋₄-acyl, C₁₋₄-alkylthio, C₁₋₄-alkylsulphinyl, C₁₋₄-alkylsulphonyl, or combinations thereof, (e.g., pyridinyl, thiazolyl, indolyl, thienyl, pyrimidinyl),
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof (e.g., benzyl),
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   aryloxy having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof (e.g., phenoxy),
   heteroaryloxy having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₇₋₁₆ arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   O-heterocyclic group, in which the heterocyclic group is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, (e.g., tetrahydrofuranyloxy);
   O-heterocyclicalkyl group, in which the heterocyclic group is nonaromatic, having 5 to 10 ring atoms in which at least 1 ring atom is a heteroatom (preferably 1 to 4 heteroatoms, preferably the heteroatoms are selected from N, S, and O), and the alkyl portion has 1 to 3 carbon atoms and the heterocyclic group is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, (e.g., morpholinylethoxy);
   or
   halogen (preferably F), hydroxy, C₁₋₄-alkoxy (e.g., OCH₃), C₁₋₄-alkyloxyC₁₋₄-alkoxy (e.g., methoxyethoxy (—OCH₂CH₂OCH₃)), C₄₋₁₂-cycloalkylalkyloxy (e.g., O-cyclopropylmethyl), C₁₋₄-alkyloxyC₇₋₁₆-arylalkyloxy (e.g., OCH₂CH₂OCH₂C₆H₅), halogenated C₁₋₄ alkoxy (e.g., OCHF₂, OCF₃), nitro, cyano, carboxy, amino, C₁₋₄ alkylamino, di-C₁₋₄-alkylamino, C₁₋₄-hydroxyalkyl, C₂₋₄-hydroxyalkoxy (e.g., OCH₂CH₂OH), —COR¹², —COOR¹², —OCOR¹², C₁₋₄-alkylthio, C₁₋₄-alkylsulphinyl, C₁₋₄-alkylsulphonyl, —SO₂NHR¹² (e.g., SO₂NHCH₃), —SO₂NHR²⁵ (e.g., SO₂NH-cyclopropylmethyl), —SO₂NR¹⁹R²⁵ (e.g., SO₂N(CH₃)₂), —SO₂R³² (e.g., —SO₂-piperidine), —SO₂-pyrrolidine)), —NHSO₂R¹², —NR¹²COR¹², —CONHR¹² (e.g., CONH-alkyl, such as CONHCH₂CH₃, CONHCH₂CH(CH₃)₂ [66266], CONH-cycloalkyl, such as CONH-cyclopropyl), —CONR¹²R²⁵ (e.g., CON(CH₂CH₃)₂, C₁₋₄ alkyl—CONR¹²R²⁵, —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₇₋₁₆ arylalkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
R¹⁹ is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
R²⁵ is H,
   alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
   cycloalkyl having 3 to 10, preferably 3 to 8 carbon atoms, which is unsubstituted or substituted one or more times with halogen, C₁₋₄-alkyl, C₁₋₄-alkoxy, oxo, or combinations thereof (e.g., cyclopropyl),
   cycloalkylalkyl having 4-12 carbon atoms which is unsubstituted or substituted one or more times with halogen, C₁₋₄-alkyl, C₁₋₄-alkoxy, oxo, or combinations thereof (e.g., cyclopropylmethyl),
   heterocyclic group which is saturated, partially saturated, or unsaturated, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof (e.g., pyrrolidinyl, piperidinyl); or
   heterocyclicalkyl group wherein the heterocyclic group has 5 to 10 ring atoms in which at least 1 ring atom is a heteroatom (preferably 1 to 4 heteroatoms, preferably the heteroatoms are selected from N, S, and O) and the alkyl portion has 1 to 4 carbon atoms, the heterocyclic group is unsubstituted or is substituted one or more times by halogen C₆₋₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof (e.g., morpholinylethyl);
R³⁰ and R³¹ are, in each case, independently
   H or alkyl having 1 to 8, preferably 1 to 4 carbon atoms, cycloalkyl having 3 to 12, preferably 3 to 8 carbon atoms or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or combinations thereof; or
   R³⁰ and R³¹ form a cycloalkyl group, spiro or fused, having 3 to 8 carbon atoms, or
   R³⁰ and R³¹ and the carbon atom to which they are attached form a C(═O) group;
R³² is a heterocyclic group which is saturated or partially saturated and has 5 to 10 ring atoms in which at least 1 ring atom is a heteroatom (preferably 1 to 4 heteroatoms, preferably the heteroatoms are selected from N, S, and O) and which is unsubstituted or substituted one or more times by halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof;

and pharmaceutically acceptable salts or solvates (e.g., hydrates) thereof, or solvates of pharmaceutically acceptable salts thereof.

According to a further method aspect, the invention includes administering to a patient a compound selected from subgerenric formulas I (a) and II (a) which correspond to formulas I and II, respectively, but in which R¹—R³ and R¹⁵—R¹⁸ are defined as follows:
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from: 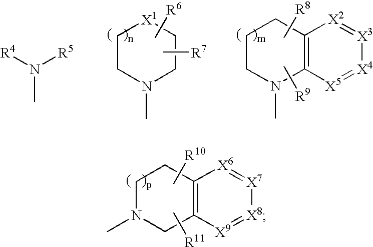
n is 0, 1, 2, or 3;
m is 0, 1, 2, or 3;
p is 0, 1, 2, or 3;
R⁴ and R⁵ are each independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently
   H,
straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR²COR¹², —CONHR¹², —NHCONHR¹², —OCONHR¹², —NHCOOR¹², —SCONHR¹², —SCSNHR¹², or —NHCSNHR¹², or combinations thereof, or
   R⁶ and R⁷ optionally form a cycloalkyl group, spiro or fused, having 3 to 8 carbon atoms,
X¹ is O, S, NR¹³, CH₂, CHR⁶ or CR⁶R⁷;
X², X³, X⁴, X⁵, X⁶, X⁷, X⁸, and X⁹ are each independently N or CR¹⁴, and wherein two adjacent X²—X⁹ groups (e.g., X⁷ and X⁸) can together be a methylenedioxy, ethylenedioxy group, difluoromethylenedioxy, or tetrafluoromethylenedioxy, to form a fused ring structure;
R⁸ and R⁹ are in each case independently
   H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof, or
   R⁸ and R⁹ form a cycloalkyl group, spiro or fused, having 3 to 8 carbon atoms;
R¹⁰ and R¹¹ are in each case independently
   H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof, or
   R¹⁰ and R¹¹ form a cycloalkyl group, spiro or fused, having 3 to 8 carbon atoms;
R¹² is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;
R¹³ H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—,
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   —CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R¹⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen (preferably F), hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄alkyl, halogenated C₁₋₄ alkyl (e.g., trifluoromethyl), hydroxy, C₁₋₄alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
R¹⁹ is H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof;

and pharmaceutically acceptable salts or solvates (e.g., hydrates) thereof, or solvates of pharmaceutically acceptable salts thereof.

According to a further method aspect, the invention includes administering to a patient a compound selected from formula I.

According to a further method aspect, the invention includes administering to a patient a compound selected from formula I wherein said compound is not

4-(4-methoxyanilino)-6,7-dimethoxycinnoline,

4-(4-ethoxyanilino)-6,7-dimethoxycinnoline,

4-(4-methylanilino)-6,7-dimethoxycinnoline,

4-(3,4-dimethylanilino)-6,7-dimethoxycinnoline,

4-(3-chloroanilino)-6,7-dimethoxycinnoline,

4-(2-chloroanilino)-6,7-dimethoxycinnoline,

4-(4-chloroanilino)-6,7-dimethoxycinnoline,

4-(3 -bromoanilino)-6,7-dimethoxycinnoline,

4-(3-hydroxy-4-methylanilino)-6,7-dimethoxycinnoline,

4-(2-fluoro-5-hydroxy-4-methylanilino)-6,7-dimethoxycinnoline,

4-(4-chloro-2-fluoro-5-hydroxyanilino)-6,7-dimethoxycinnoline,

6,7-dimethoxy-4-(1-piperazinyl)cinnoline,

4-amino-6,7-dimethoxycinnoline,

4-anilino-6,7-dimethoxycinnoline,

or a pharmaceutically acceptable salt thereof, or a solvate thereof, or a solvate of a pharmaceutically acceptable salt thereof.

According to a further method aspect, the invention includes administering to a patient a compound selected from formula I(a).

According to a further method aspect, the invention includes administering to a patient a compound selected from formula I(a) wherein said compound is not

4-(4-methoxyanilino)-6,7-dimethoxycinnoline,

4-(4-ethoxyanilino)-6,7-dimethoxycinnoline,

4-(4-methylanilino)-6,7-dimethoxycinnoline,

4-(3,4-dimethylanilino)-6,7-dimethoxycinnoline,

4-(3-chloroanilino)-6,7-dimethoxycinnoline,

4-(2-chloroanilino)-6,7-dimethoxycinnoline,

4-(4-chloroanilino)-6,7-dimethoxycinnoline,

4-(3-bromoanilino)-6,7-dimethoxycinnoline,

4-(3-hydroxy-4-methylanilino)-6,7-dimethoxycinnoline,

4-(2-fluoro-5-hydroxy-4-methylanilino)-6,7-dimethoxycinnoline,

4-(4-chloro-2-fluoro-5-hydroxyanilino)-6,7-dimethoxycinnoline,

6,7-dimethoxy-4-(1-piperazinyl)cinnoline,

4-amino-6,7-dimethoxycinnoline,

4-anilino-6,7-dimethoxycinnoline,

or a pharmaceutically acceptable salt thereof, or a solvate thereof, or a solvate of a pharmaceutically acceptable salt thereof.

According to a further method aspect, the compound administered is selected from Formula I or Formula I(a) wherein when n is 1 and X¹ is NH, R⁶ and R⁷ are not both H.

According to a further method aspect, the compound administered is selected from Formula I or Formula I(a) wherein when one of R⁴ or R⁵ is H, unsubstituted phenyl, or phenyl substituted by alkyl, hydroxyl and/or halogen, the other is not H.

According to a further method aspect, the compound administered is selected from Formula I or Formula I(a) wherein when n is 1 and X¹ is NH, R⁶ and R⁷ are not both H, and when one of R⁴ or R⁵ is H, unsubstituted phenyl, or phenyl substituted by alkyl, hydroxyl and/or halogen, the other is not H.

According to a further method aspect, the compound administered is selected from Formula I or Formula I(a) wherein when one of R⁴ and R⁵ is H or substituted or unsubstituted phenyl, the other is not H.

According to a further method aspect, the compound administered is selected from Formula I or Formula I(a) wherein when n is 1 and X¹ is NH, R⁶ and R⁷ are not both H, and when one of R⁴ and R⁵ is H or substituted or unsubstituted phenyl, the other is not H.

According to a further method aspect, the compound administered is selected from Formula I or Formula I(a) wherein —NR⁴R⁵ is not NH₂, NHCH₃, or substituted or unsubstituted anilino.

According to a further method aspect, the compound administered is selected from Formula I or Formula I(a) wherein —NR⁴R⁵ is not NH₂, unsubstituted monoalkylamino, or substituted or unsubstituted anilino.

According to a further method aspect, the compound administered is selected from Formula I or Formula I(a) wherein —NR⁴R⁵ is not NH₂, unsubstituted monoalkylamino, unsubstituted dialkylamino, or substituted or unsubstituted anilino.

According to a further method aspect, the invention includes administering to a patient a compound selected from Formula II.

According to a further method aspect, the invention includes administering to a patient a compound selected from Formula II wherein said compound is not

6,7-dimethoxy-α-1-naphthyl-4-cinnoline-acetonitrile,

4-(p-aminobenzyl)-6,7-dimethoxy-cinnoline,

6,7-dimethoxy-α-(m-methoxyphenyl)-4-cinnoline-acetonitrile,

α-[4,5-dihydro-4,4-dimethyl-1-(1-methylethyl)-1H-imidazol-2-yl]-6,7-dimethoxy-4-cinnolineacetonitrile,

α-(3,4-dimethoxyphenyl)-6,7-dimethoxy-4-cinnoline-acetamide,

6,7-dimethoxy-α-phenyl-4-cinnoline-acetonitrile,

α-(3,4-dimethoxyphenyl)-6,7-dimethoxy-4-cinnoline-acetonitrile,

6,7-dimethoxy-α-(p-iodophenyl)-4-cinnoline-acetonitrile,

6,7-dimethoxy-α-(p-bromophenyl)-4-cinnoline-acetonitrile,

α-(3,4-dichlorophenyl)-6,7-dimethoxy-4-cinnoline-acetonitrile,

6,7-dimethoxy-α-(phenyl)-4-cinnoline-acetamide (also called α-(6,7-dimethoxy-4-cinnolyl)phenylacetamide),

6,7-dimethoxy-α-(4-chlorophenyl)-4-cinnoline-acetonitrile,

α-(4-aminophenyl)-6,7-dimethoxy-4-cinnoline-acetonitrile, or

4-benzyl-6,7-dimethoxycinnoline,

or a pharmaceutically acceptable salt thereof, or a solvate thereof, or a solvate of a pharmaceutically acceptable salt thereof.

According to a further method aspect, the invention includes administering to a patient a compound selected from Formula II(a).

According to a further method aspect, the invention includes administering to a patient a compound selected from Formula II(a) wherein said compound is not

6,7-dimethoxy-α-1-naphthyl-4-cinnoline-acetonitrile,

4-(p-aminobenzyl)-6,7-dimethoxy-cinnoline,

6,7-dimethoxy-α-(m-methoxyphenyl)-4-cinnoline-acetonitrile,

α-[4,5-dihydro-4,4-dimethyl-1-(1-methylethyl)-1H-imidazol-2-yl]-6,7-dimethoxy-4-cinnolineacetonitrile,

α-(3,4-dimethoxyphenyl)-6,7-dimethoxy-4-cinnoline-acetamide,

6,7-dimethoxy-α-phenyl-4-cinnoline-acetonitrile,

α-(3,4-dimethoxyphenyl)-6,7-dimethoxy-4-cinnoline-acetonitrile,

6,7-dimethoxy-α-(p-iodophenyl)-4-cinnoline-acetonitrile,

6,7-dimethoxy-α-(p-bromophenyl)-4-cinnoline-acetonitrile,

α-(3,4-dichlorophenyl)-6,7-dimethoxy-4-cinnoline-acetonitrile,

6,7-dimethoxy-α-(phenyl)-4-cinnoline-acetamide (also called α-(6,7-dimethoxy-4-cinnolyl)phenylacetamide),

6,7-dimethoxy-α-(4-chlorophenyl)-4-cinnoline-acetonitrile,

α-(4-aminophenyl)-6,7-dimethoxy-4-cinnoline-acetonitrile, or

4-benzyl-6,7-dimethoxycinnoline,

or a pharmaceutically acceptable salt thereof, or a solvate thereof, or a solvate of a pharmaceutically acceptable salt thereof.

According to a further method aspect, the invention includes administering to a patient a compound of Formula II or Formula II(a), wherein when R¹⁸ is cyano, then R¹⁷ is other than halo-substituted phenyl.

According to a further method aspect, the invention includes administering to a patient a compound of Formula II or Formula II(a), wherein R¹⁸ is other than H.

According to a further method aspect, the invention includes administering to a patient a compound of Formula II or Formula II(a), wherein R¹⁸ is not H, cyano, or —CONHR¹⁹.

According to a further method aspect, the invention includes administering to a patient a compound selected from Formula III
wherein 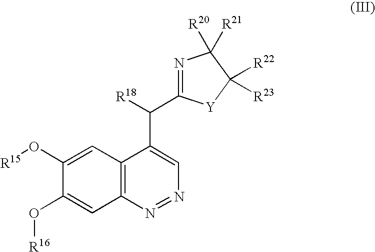
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁸ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
Y is NR²⁴ O or S;
R²⁰, R^{21,} R²², and R²³ are independently
   H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₁₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof, or
   cyano, carboxy, C₁₋₄-hydroxyalkyl, COR¹⁹, COOR¹⁹, CONHR¹⁹ or combinations thereof,
   wherein two of R²⁰, R²¹, R²², and R²³ together may optionally form a spiro or fused cycloalkyl group having 3 to 8 carbon atoms, and R²⁰ and R²¹ or R²² and R²³ together may optionally form an oxo group;
R²⁴ is H,
   straight, branched or cyclic alkyl having up to 12 carbon atoms (e.g., cycloalkyl having 3-12 carbon atoms and cycloalkylalkyl having 4-12 carbon atoms), which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C≡C—, or
   aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   arylalkyl having 7 to 16 carbon atoms (wherein the aryl portion preferably contains 6 to 14 carbon atoms and the alkyl portion preferably contains 1 to 4 carbon atoms), which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
   heteroaryl having 5 to 10 ring atoms in which at least 1 ring atom is a heteroatom (preferably 1 to 4 heteroatoms, preferably the heteroatoms are selected from N, S, and O) which is unsubstituted or substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heteroarylalkyl wherein the heteroaryl portion has 5 to 10 ring atoms in which at least 1 ring atom is a heteroatom (preferably 1 to 4 heteroatoms, preferably the heteroatoms are selected from N, S, and O) and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
   heterocycle, which is nonaromatic, having 5 to 10 ring atoms in which at least 1 ring atom is a heteroatom (preferably 1 to 4 heteroatoms, preferably the heteroatoms are selected from N, S, and O), and is unsubstituted or is substituted one or more times by halogen, C₆₋₁₄ aryl, C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
   carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C_{1-4l alkylamino, di-C}₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;

or a pharmaceutically acceptable salt thereof, or a solvate thereof, or a solvate of a pharmaceutically acceptable salt thereof.

According to a further method aspect, the invention includes administering to a patient a compound of Formula III, wherein said compound is not α-[4,5-dihydro-4,4-dimethyl-1-(1-methylethyl)-1H-imidazol-2-yl]-6,7-dimethoxy-4-cinnolineacetonitrile.

According to a further method aspect, the invention includes administering to a patient a compound of Formula III, wherein if R24 is isopropyl, then R20 and R21 are not both methyl.

According to a further method aspect, the invention includes administering to a patient a compound of Formula III, wherein if R24 is isopropyl, then R20 and R21 are not both alkyl.

According to a further method aspect, the invention includes administering to a patient a compound of Formula III, wherein R¹⁸ is other than H.

According to a further method aspect, the invention includes administering to a patient a compound of Formula II or Formula II(a), wherein R¹⁸ is not H, cyano, or —CONHR¹⁹.

Halogen herein refers to F, Cl, Br, and I. Preferred halogens are F and Cl.

Alkyl means a straight-chain or branched-chain aliphatic hydrocarbon radical. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

These alkyl radicals can optionally have one or more —CH₂CH₂— groups replaced in each case by —CH═CH— or —C≡C— groups. Suitable alkenyl or alkynyl groups include, b u t are not limited to, 1-propenyl, 2-propenyl, 1-propynyl, 1-butenyl, 2-butenyl, 3-butenyl, 1-butynyl, 1,3-butadienyl, and 3-methyl-2-butenyl.

The alkyl groups include cycloalkyl groups, e.g., monocyclic, bicyclic or tricyclic saturated hydrocarbon radical having 3 to 8 carbon atoms, preferably 3 to 6 carbon atoms. Suitable cycloalkyl groups include, but are not limited to, cyclopropyl, cyclobutyl, cyclopentyl, cyclohexyl, cycloheptyl, cyclooctyl, and norbornyl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, and bicyclo[4.2.0]octyl.

The alkyl groups also include cycloalkylalkyl in which the cycloalkyl portions have preferably 3 to 8 carbon atoms, preferably 4 to 6 carbon atoms and alkyl the portions have preferably 1 to 8 carbon atoms, preferably 1 to 4 carbon atoms. Suitable examples include, but are not limited to, cyclopentylethyl and cyclopropylmethyl.

In the arylalkyl groups and heteroalkyl groups, “alkyl” refers to a divalent alkylene group preferably having 1 to 4 carbon atoms.

In the cases where alkyl is a substituent (e.g., alkyl substituents on aryl and heteroaryl groups) or is part of a substituent (e.g., in the alkylamino, dialkylamino, hydroxyalkyl, hydroxyalkoxy, alkylthio, alkylsulphinyl, and alkylsulphonyl substituents), the alkyl portion preferably has 1 to 12 carbon atoms, especially 1 to 8 carbon atoms, in particular 1 to 4 carbon atoms.

Aryl, as a group or substituent per se or as part of a group or substituent, refers to an aromatic carbocyclic radical containing 6 to 14 carbon atoms, preferably 6 to 12 carbon atoms, especially 6 to 10 carbon atoms. Suitable aryl groups include, but are not limited to, phenyl, naphthyl and biphenyl. Substituted aryl groups include the above-described aryl groups which are substituted one or more times by, for example, halogen, alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy).

Arylalkyl refers to an aryl-alkyl-radical in which the aryl and alkyl portions are in accordance with the previous descriptions. Suitable examples include, but are not limited to, 1-phenethyl, 2-phenethyl, phenpropyl, phenbutyl, phenpentyl, and naphthylenemethyl.

Heteroaryl groups refer to unsaturated heterocyclic groups having one or two rings and a total number of 5 to 10 ring atoms wherein at least one of the ring atoms is preferably an N, O or S atom. Preferably, the heteroaryl group contains 1 to 3, especially 1 or 2, hetero-ring atoms selected from N, O and S. Suitable heteroaryl groups include, but are not limited to, furyl, thienyl, pyrrolyl, pyrazolyl, imidazolyl, pyridyl, pyrimidinyl, indolyl, quinolinyl, naphthyridinyl, azaindolyl (e.g.,7-azaindolyl), 1,2,3,4,-tetrahydroisoquinolyl, isoxazolyl, thiazolyl, and the like. Preferred heteroaryl groups include, but are not limited to, 2-thienyl, 3-thienyl, 2-, 3- or 4-pyridyl, 2-, 3-, 4-, 5-, 6-, 7- or 8-quinolinyl, 7- azaindolyl, (1,3-thiazol-2-yl), and 1-, 3-, 4-, 5-, 6-, 7- or 8-isoquinolinyl.

Substituted heteroaryl groups refer to the heteroaryl groups described above which are substituted in one or more places by preferably halogen, aryl, alkyl, alkoxy, cyano, halogenated alkyl (e.g., trifluoromethyl), nitro, oxo, amino, alkylamino, and dialkylamino.

Heterocycles are non-aromatic, saturated or partially unsaturated, cyclic groups containing at least one hetero ring atom, preferably selected from N, S, and O, for example, 3-tetrahydrofuranyl, piperidinyl, imidazolinyl, imidazolidinyl, pyrrolinyl, pyrrolidinyl, morpholinyl, piperazinyl, oxazolidinyl, and indolinyl.

Heteroarylalkyl refers to a heteroaryl-alkyl-group wherein the heteroaryl and alkyl portions are in accordance with the previous discussions. Suitable examples include, but are not limited to, pyridylmethyl, thienylmethyl, pyrimidinylmethyl, pyrazinylmethyl, isoquinolinylmethyl, pyridylethyl and thienylethyl.

Carbocyclic structures are non-aromatic monocyclic or bicyclic structures containing 5 to 14 carbon atoms, preferably 6 to 10 carbon atoms, wherein the ring structure(s) optionally contain at least one C═C bond. Suitable examples include, but are not limited to, cyclopentenyl, cyclohexenyl, tetrahydronaphthenyl and indan-2-yl.

Acyl refers to alkanoyl radicals having 2 to 4 carbon atoms. Suitable acyl groups include, but are not limited to, formyl, acetyl, propionyl, and butanoyl.

Substituted radicals preferably have 1 to 3 substituents, especially 1 or 2 substituents.

R¹ and R² are each preferably alkyl having 1 to 4 carbon atoms, which is unsubstituted or substituted one or more times by halogen, e.g., CH₃, CHF₂, CF₃, especially CH₃.

R³ is preferably

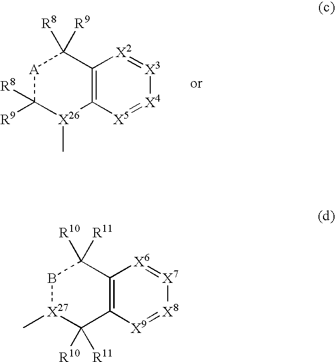

in which X², X³, X⁴, X⁵, X⁶, X⁷, X⁸, and X⁹ are each preferably CR¹⁴, and R¹⁴ is preferably H, CH₃, CN, F, CF₃, OCH₂-cyclopropyl, OCH₃, OC₂H₅, CH₂OH, OCH₂CH₂OH, OCH₂CH₂OCH₃, SO₂NHCH₃, SO₂NHCH₂-cyclopropyl, SO₂N(CH₃)₂, heterocyclic group (e.g., pyridyl (e.g., 4-pyridyl), thiazolyl, furyl, thienyl), or CO₂CH₃.

X²⁶ is preferably N or CR⁸, more preferably N. X²⁷ is preferably N, CH, or CR¹⁰, more preferably N.

R⁸, R⁹, R¹⁰, and R¹¹ are each preferably H or CH₃, especially H.

In another preferred embodiment, R³ is of formula (c), one set of R⁸ and R⁹ together with the carbon to which they are attached form a C(═O) group. In a further preferred embodiment R³ is of formula (d) and one set of R¹⁰ and R¹¹ together with the carbon to which they are attached form a C(═O) group.

In another embodiment, the invention includes compounds of Formula I in which R³ is of formula (c) or (d) and R¹⁴ is H, halogen, alkoxy, alkoxyalkyl, cycloalkylalkyloxy, or alkyloxyalkoxy.

In a further preferred embodiment, -A- represents a single bond, a double bond, —CR⁸R⁹—, ═CR⁸—, or —CR⁸═, more preferably a single bond or —CR⁸R⁹—.

In a further preferred embodiment, —B— represents a single bond, —CR¹⁰R¹¹—, or —CR¹⁰═, more preferably a single bond or —CR¹⁰R¹¹—.

For compounds in which R³ is represented by a formula other than (a) or (b), then:

-D- is preferably a single bond, a double bond, —CR²⁶R²⁷—, ═CR²⁶—, or —CR²⁶═, more preferably —CR²⁶R²⁷—.

-E- is preferably a single bond, —CR²⁸R²⁹—, or —CR²⁸═, more preferably —CR²⁸R²⁹—.

R²⁶, R²⁷, R²⁸, R²⁹, R³⁰, and R³¹ are each preferably H or CH₃, especially H.

In another preferred embodiment, R³⁰ and R³¹ together with the carbon to which they are attached form a C(═O) group.

In a further preferred embodiment, formula (h) contains no double bonds or two non-adjacent double bonds. When formula (h) contains two non-adjacent double bonds (i) there is a double bond between X²¹ and X²² and a double bond between X²³ and X²⁴, (ii) there is a double bond between X²² and X²³ and a double bond between X²⁴ and X²⁵, (iii) there is a double bond between X²¹ and X²⁵ and a double bond between X²² and X²³, or (iv) there is a double bond between X²¹ and X²⁵ and a double bond between X²³ and X²⁴.

In compounds of Formula I in which R³ is represented by Formula (f), preferred compounds include those in which -E- is CR²⁸R²⁹—, R²⁸ and R²⁹ are H, X¹³ and X¹⁴ are N, and X¹⁵ is CR¹⁴ (e.g., R¹⁴ is carboxy, CO₂R¹² (e.g., CO₂CH₃, CO₂CH₂CH₃), CONHR¹² (e.g., CONH-cyclopropyl, CONH-cyclopropylmethyl).

In compounds of Formula I in which R³ is represented by Formula (g), preferred compounds include those in which (i) X¹⁶, X¹⁷, X¹⁸, X¹⁹, and X²⁰ are C or CR¹⁴, and (ii) one of X¹⁶, X¹⁷, X¹⁸, X¹⁹, and X²⁰ is N and the rest are C or CR¹⁴.

Additional preferred compounds in which R³ is represented by Formula (g) also include those in which:
(a) X¹⁶, X¹⁷, X¹⁸ and X²⁰ are CR¹⁴ (e.g., CH) and X¹⁹ is N;
(b) X¹⁶, X¹⁷, X¹⁸ and X²⁰ are CH and X¹⁹ is CR¹⁴ (e.g., R¹⁴ is CONHR¹² (e.g., CONHCH₂CH₃, CONHCH(CH₃)₂, CONH-cyclopropyl, CONH-cyclohexyl), CONHR¹²R²⁵ (e.g., CON(CH₃)₂)); and
(c) X¹⁶, X¹⁷ and X²⁰ are CR¹⁴ (e.g., CH) and X¹⁸ and X¹⁹ form a fused aryl (e.g., unsubstituted phenyl or substituted phenyl (e.g., methoxyphenyl)).

In compounds of Formula I in which R³ is represented by Formula (h), preferred compounds include:
(a) compounds wherein X²³ is N or NR¹⁴, X²⁵ is N, and X²¹, X²² and X²⁴ are C or CHR¹⁴ (e.g., X²² and X²⁴ are CH₂, X²³ is NR¹⁴, X²¹ is N, X²¹ is C, and R³⁰ and R³¹, together with X²¹ form a C(═O) group;
(b) X²² and X²³ are N or NR¹⁴ and X²¹, X²⁴, and X²⁵ are C or CR¹⁴ (e.g., X²² is N, X²³ is NR¹⁴, X²⁵ is C, and X²¹ and X²⁴ are CH); and
(c) X²¹ is S, X²⁴ is N, X²² and X²³ are CR¹⁴ (e.g., CH) and X²⁵ is C.

R¹⁵ and R¹⁶ are each preferably alkyl having 1 to 4 carbon atoms, which is unsubstituted or substituted one or more times by halogen, especially CH₃.

R¹⁸ is preferably CN.

In Formula III, Y is preferably NR²⁴ or O, and R²⁰ and R²¹ are each preferably H, CH₃ or phenyl. R²² and R²³ are each preferably H or CH₃, especially H.

In Formula III, R²⁴ is preferably cyclopropyl, benzyl or cyclopropylmethyl.

According to a compound and/or method aspect of the invention, the compounds of the invention are selected from:
1) 6,7-Dimethoxy-4-[7-(trifluoromethyl)-3,4-dihydroisoquinolin-2(1H)-yl]cinnoline,
2) 6,7-Dimethoxy-4-(5-methyl-2,3 -dihydro-1H-indol-1-yl)cinnoline,
3) 6,7-Dimethoxy-4-[7-(trifluoromethyl)-3,4-dihydroquinolin-1(2H)-yl]cinnoline,
4) 6,7-Dimethoxy-4-(6-methyl-3,4-dihydroisoquinolin-2(1H)-yl)cinnoline,
5) 6,7-Dimethoxy-4-(8-methyl-3,4-dihydroisoquinolin-2(1H)-yl)cinnoline,
6) 4-(6,8-Dimethoxy-3,4-dihydroisoquinolin-2(1H)-yl)-6,7-dimethoxycinnoline,
7) 6,7-Dimethoxy-4-(6-methyl-3,4-dihydroquinolin-1(2H)-yl)cinnoline,
8) 4-(6,7-Dimethoxy-3-methyl-3,4-dihydroisoquinolin-2(1H)-yl)-6,7-dimethoxycinnoline,
9) 4-(6,7-Dimethoxy-3,4-dihydroisoquinolin-2(1H)-yl)-6,7-dimethoxycinnoline hydrochloride,
10) 4-(5,7-Dimethoxy-3,4-dihydroisoquinolin-2(1H)-yl)-6,7-dimethoxycinnoline,
11) 6,7-Dimethoxy-4-(5-methyl-3,4-dihydroisoquinolin-2(1H)-yl)cinnoline,
12) 6,7-Dimethoxy-4-(7-methoxy-3,4-dihydroisoquinolin-2(1H)-yl)cinnoline,
13) 6,7-Dimethoxy-4-(7-methyl-3,4-dihydroisoquinolin-2(1H)-yl)cinnoline,
14) 6,7-Dimethoxy-4-(5-methoxy-3,4-dihydroisoquinolin-2(1H)-yl)cinnoline,
15) Methyl 2-(6,7-dimethoxycinnolin-4-yl)-1,2,3,4-tetrahydroisoquinoline-6-carboxylate,
16) 4-(6,7-Dihydrothieno[3,2-c]pyridin-5(4H)-yl)-6,7-dimethoxycinnoline,
17) 6,7-Dimethoxy-4-[7-(2-methoxyethoxy)-3,4-dihydroisoquinolin-2(1H)-yl]cinnoline,
18) 2-(6,7-Dimethoxycinnolin-4-yl)-7-methoxy-3,4-dihydroisoquinolin-1(2H)-one,
19) 6,7-Dimethoxy-4-(6-nitro-2,3-dihydro-1H-indol-1-yl)cinnoline,
20) Tert-butyl [1-(6,7-dimethoxycinnolin-4-yl)-1,2,3,4-tetrahydroquinolin-6-yl]carbamate,
21) 6,7-Dimethoxy-4-(5-nitro-2,3-dihydro-1H-indol-1-yl)cinnoline,
22) 4-(5-Fluoro-2,3-dihydro-1H-indol-1-yl)-6,7-dimethoxycinnoline,
23) 1-(6,7-Dimethoxycinnolin-4-yl)-N,N-dimethylindoline-5-sulfonamide,
24) 6,7-Dimethoxy-4-(6-pyridin-4-yl-3,4-dihydroisoquinolin-2(1H)-yl)cinnoline,
25) 6,7-Dimethoxy-4-(7-phenoxy-3,4-dihydroisoquinolin-2(1H)-yl)cinnoline,
26) 4-(6,7-Dimethoxy-3,4-dihydroquinolin-1(2H)-yl)-6,7-dimethoxycinnoline,
27) 2-(6,7-Dimethoxycinnolin-4-yl)-8-fluoro-7-methoxy-3,4-dihydroisoquinolin-1(2H)-one,
28) 2-(6,7-Dimethoxycinnolin-4-yl)-6-fluoro-3,4-dihydroisoquinolin-1(2H)-one,
29) 2-(6,7-Dimethoxycinnolin-4-yl)-7-fluoro-6-methoxy-3,4-dihydroisoquinolin-1(2H)-one,
30) 7-(Cyclopropylmethoxy)-2-(6,7-dimethoxycinnolin-4-yl)-6-methoxy-3,4-dihydroisoquinolin-1(2H)-one,
31) 2-(6,7-Dimethoxycinnolin-4-yl)-8-methoxy-2,3,4,5-tetrahydro-1H-2-benzazepin-1-one,
32) 2-(6,7-Dimethoxycinnolin-4-yl)-7,8-dimethoxy-3,4-dihydroisoquinolin-1(2H)-one,
33) 6-(Cyclopropylmethoxy)-2-(6,7-dimethoxycinnolin-4-yl)-7-methoxy-3,4-dihydroisoquinolin-1(2H)-one,
34) 4-(5,6-Dimethoxy-2,3-dihydro-1H-indol-1-yl)-6,7-dimethoxycinnoline,
35) 6,7-Dimethoxy-4-[6-(1,3-thiazol-2-yl)-3,4-dihydroisoquinolin-2(1H)-yl]cinnoline,
36) 1-(6,7-Dimethoxycinnolin-4-yl)-N,N-diethylindoline-5-sulfonamide,
37) N-(Cyclopropylmethyl)-1-(6,7-dimethoxycinnolin-4-yl)indoline-5-sulfonamide,
38) 1-(6,7-Dimethoxycinnolin-4-yl)-N-methylindoline-5-sulfonamide,
39) 1-(6,7-Dimethoxycinnolin-4-yl)-N,N-dimethyl-1,2,3,4-tetrahydroquinoline-5-sulfonamide,
40) 2-(6,7-Dimethoxycinnolin-4-yl)-6,7-dimethoxy-3,4-dihydroisoquinolin-1(2H)-one,
41) 4-(2,3-Dihydro-1H-indol-1-yl)-6,7-dimethoxycinnoline,
42) 6,7-Dimethoxy-4-[5-(methylsulfonyl)-2,3-dihydro-1H-indol-1-yl]cinnoline,
43) 4-[5-(3-Furyl)-2,3-dihydro-1H-indol-1-yl]-6,7-dimethoxycinnoline,
44) 4-(1H-Indol-1-yl)-6,7-dimethoxycinnoline,
45) 4-(1-Benzyl-1H-pyrazol-4-yl)-6,7-dimethoxycinnoline,
46) 6,7-Dimethoxy-4-pyridin-3-ylcinnoline,
47) 6,7-Dimethoxy-4-[5-(3-thienyl)-2,3 -dihydro-1H-indol-1-yl]cinnoline,
48) 6,7-Dimethoxy-4-(5-pyrimidin-5-yl-2,3-dihydro-1H-indol-1-yl)cinnoline,
49) 6,7-Dimethoxy-4-(1,3-thiazol-2-yl)cinnoline,
50) 1-(6,7-Dimethoxy-1-naphthyl)-N-ethylindoline-5-sulfonamide hydroformate,
51) 1-(6,7-Dimethoxy-1-naphthyl)-N-isopropylindoline-5-sulfonamide hydroformate,
52) N-cyclopropyl-1-(6,7-dimethoxycinnolin-4-yl)indoline-5-sulfonamide,
53) 6,7-dimethoxy-4-[5-(pyrrolidin-1-ylsulfonyl)-2,3-dihydro-1H-indol-1-yl]cinnoline,
54) 1-(6,7-dimethoxycinnolin-4-yl)-N,N-diisopropylindoline-5-sulfonamide,
55) 1-(6,7-dimethoxycinnolin-4-yl)-N-(2-methoxyethyl)indoline-5-sulfonamide,
56) 1-(6,7-dimethoxycinnolin-4-yl)-N-(2-morpholin-4-ylethyl)indoline-5-sulfonamide,
57) 6,7-dimethoxy-4-(5-pyridin-4-yl-2,3 -dihydro-1H-indol-1-yl)cinnoline,
58) 6,7-dimethoxy-4-[7-(2-methoxyethoxy)-3,4-dihydroisoquinolin-2(1H)-yl]cinnoline hydrochloride,
59) 4-[5-(3,5-dimethylisoxazol-4-yl)-2,3-dihydro-1H-indol-1-yl]-6,7-dimethoxycinnoline,
60) 6,7-dimethoxy-4-(6-methoxy-2-naphthyl)cinnoline,
61) 6,7-dimethoxy-4-[5-(piperidin-1-ylsulfonyl)-2,3-dihydro-1H-indol-1-yl]cinnoline,
62) 6,7-dimethoxy-N-(5-methyl-4H-pyrazol-3-yl)cinnolin-4-amine hydroformate,
63) 6,7-dimethoxy-N-(4-methyl-1,3-thiazol-2-yl)cinnolin-4-amine hydroformate,
64) 3-(6,7-dimethoxycinnolin-4-yl)-N-ethylbenzamide,
65) 3-(6,7-dimethoxycinnolin-4-yl)-N-isobutylbenzamide,
66) N-cyclopropyl-3-(6,7-dimethoxycinnolin-4-yl)benzamide,
66) 6,7-bis(difluoromethoxy)-4-[7-(2-methoxyethoxy)-3,4-dihydroisoquinolin-2(1H)-yl]cinnoline,
68) 2-(6,7-dimethoxycinnolin-4-yl)-6,7-dimethoxy-1,4-dihydroisoquinolin-3(2H)-one,
69) 6-(benzyloxy)-2-(6,7-dimethoxycinnolin-4-yl)-3,4-dihydroisoquinolin-1(2H)-one,
70) 2-(6,7-dimethoxycinnolin-4-yl)-5-hydroxy-3,4-dihydroisoquinolin-1(2H)-one,
71) 6,7-dimethoxy-4-[6-(2-methoxyethoxy)-3,4-dihydroisoquinolin-2(1H)-yl]cinnoline,
72) 6,7-bis(difluoromethoxy)-4-[7-(2-methoxyethoxy)-3,4-dihydroisoquinolin-2(1H)-yl]cinnoline hydroformate,
73) N-cyclohexyl-3-(6,7-dimethoxycinnolin-4-yl)benzamide,
74) 3-(6,7-dimethoxycinnolin-4-yl)-N,N-diethylbenzamide,
75) 2-(6,7-dimethoxycinnolin-4-yl)-5-(2-methoxyethoxy)-3,4-dihydroisoquinolin-1(2H)-one,
76) 4-(3,4-dihydronaphthalen-2-yl)-6,7-dimethoxycinnoline hydroformate,
77) 6,7-dimethoxy-4-[7-(tetrahydrofuran-3-yloxy)-3,4-dihydroisoquinolin-2(1H)-yl]cinnoline,
78) 6,7-dimethoxy-4-[7-(2-morpholin-4-ylethoxy)-3,4-dihydroisoquinolin-2(1H)-yl]cinnoline,
79) 2-{[2-(6,7-dimethoxycinnolin-4-yl)-1,2,3,4-tetrahydroisoquinolin-5-yl]oxy}ethanol,
80) 4-[7-[2-(benzyloxy)ethoxy]-3,4-dihydroisoquinolin-2(1H)-yl]-6,7-dimethoxycinnoline,
81) 2-(6,7-dimethoxycinnolin-4-yl)-1,2,3,4-tetrahydroisoquinoline-5-carboxylic acid hydrochloride, and
82) 6,7-dimethoxy-4-[8-(2-methoxyethoxy)-3,4-dihydroisoquinolin-2(1H)-yl]cinnoline,

wherein salts listed above can also be in free base form or in the form of another pharmaceutically acceptable salt, and free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

The following table presents the structures for selected compounds of Formula I in accordance with the present invention:

| Compound | Structure |
|---|---|
| 1) | 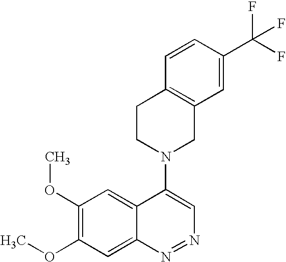 |
| 2) | 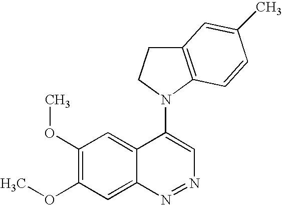 |
| 3) | 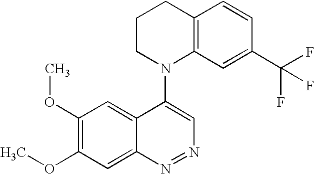 |
| 4) | 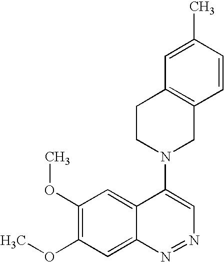 |
| 5) | 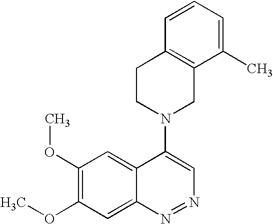 |
| 6) | 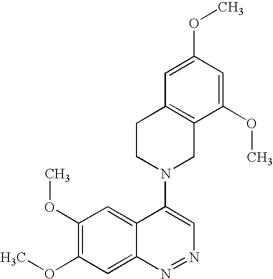 |
| 7) | 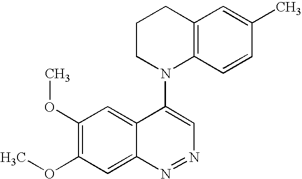 |
| 8) | 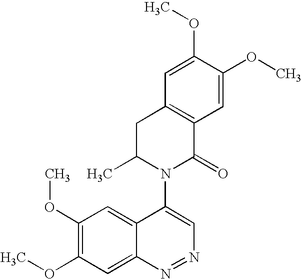 |
| 9) | 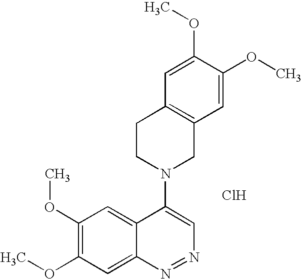 |
| 10) | 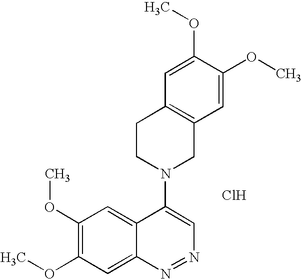 |
| 11) | 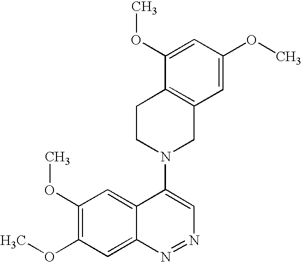 |
| 12) | 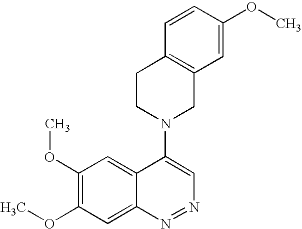 |
| 13) | 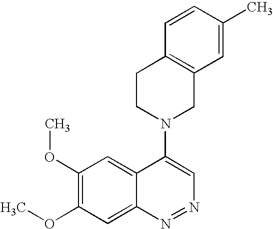 |
| 14) | 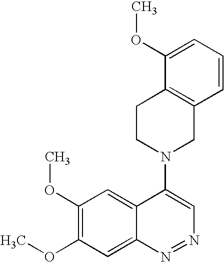 |
| 15) | 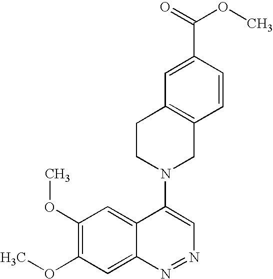 |
| 16) | 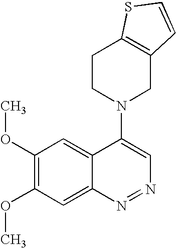 |
| 17) | 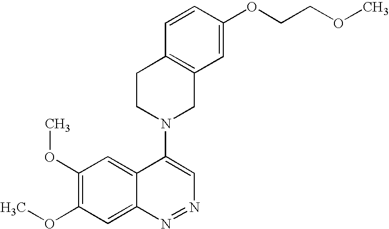 |
| 18) | 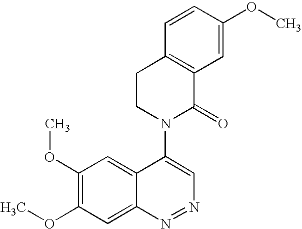 |
| 19) | 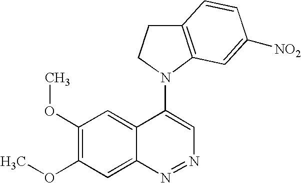 |
| 20) | 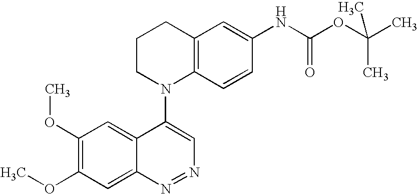 |
| 21) | 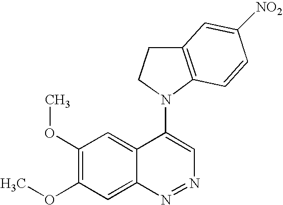 |
| 22) | 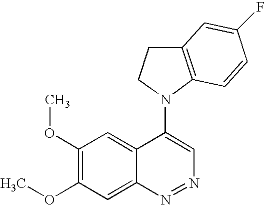 |
| 23) | 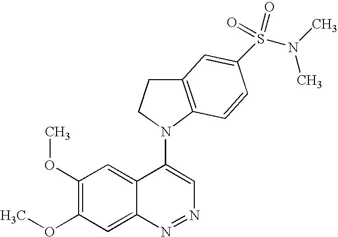 |
| 24) | 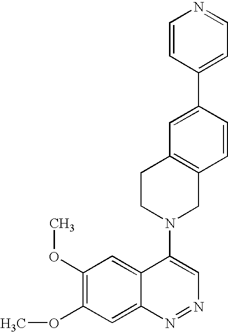 |
| 25) | 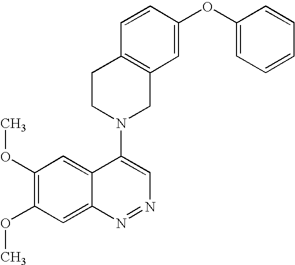 |
| 26) | 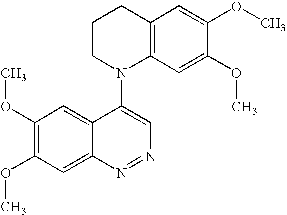 |
| 27) | 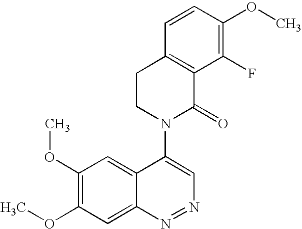 |
| 28) | 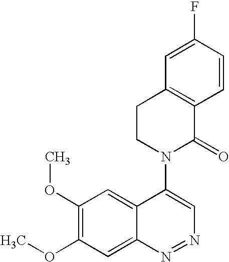 |
| 29) | 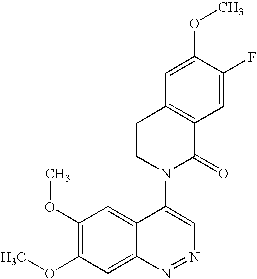 |
| 30) | 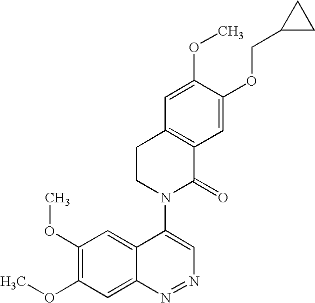 |
| 31) | 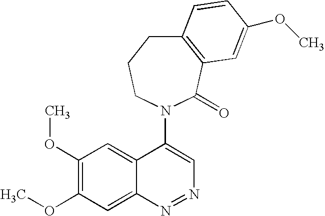 |
| 32) | 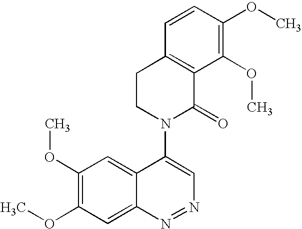 |
| 33) | 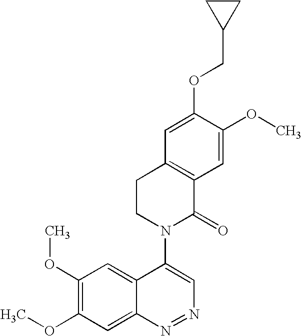 |
| 34) | 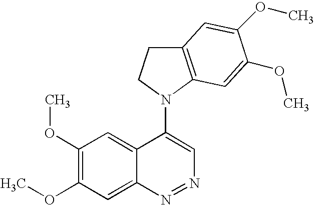 |
| 35) | 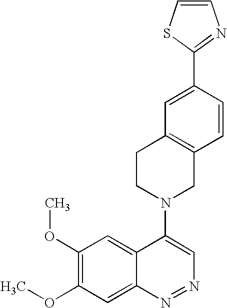 |
| 36) | 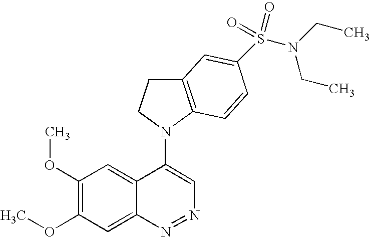 |
| 37) | 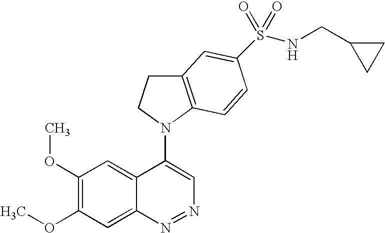 |
| 38) | 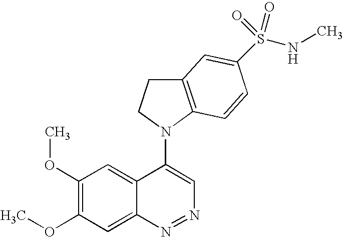 |
| 39) | 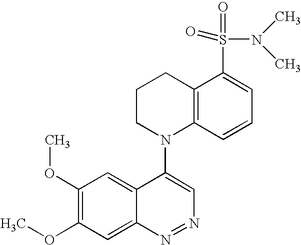 |
| 40) | 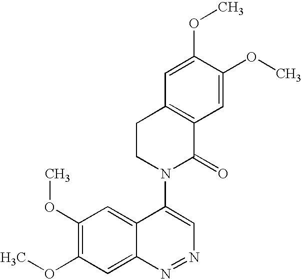 |
| 41) | 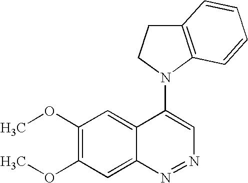 |
| 42) | 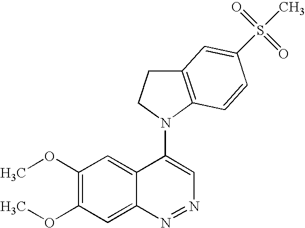 |
| 43) | 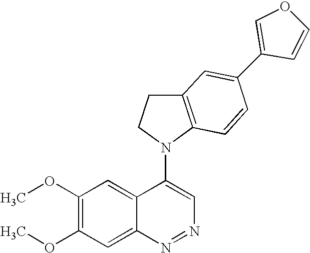 |
| 44) | 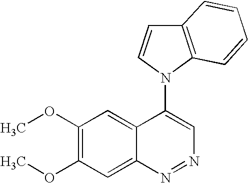 |
| 45) | 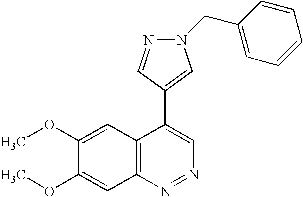 |
| 46) | 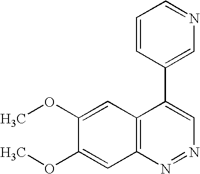 |
| 47) | 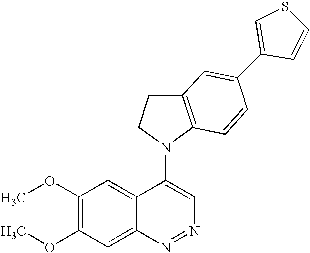 |
| 48) | 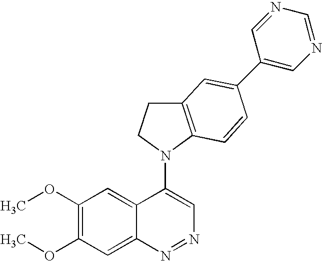 |
| 49) | 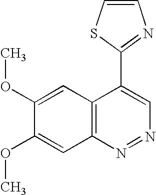 |
| 50) | 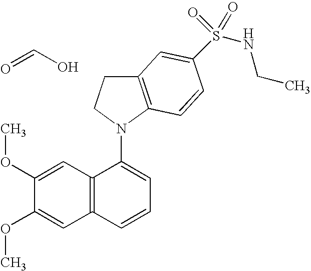 |
| 51) | 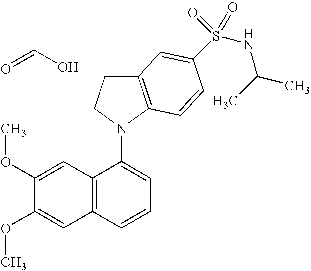 |
| 52) | 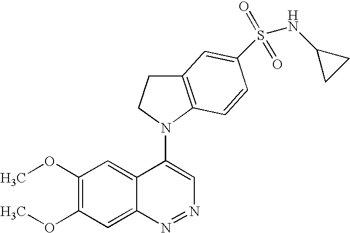 |
| 53) | 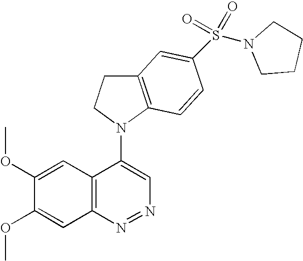 |
| 54) | 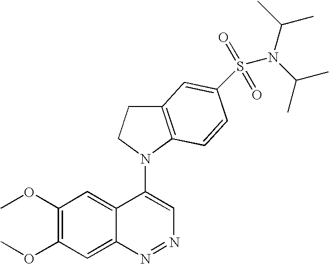 |
| 55) | 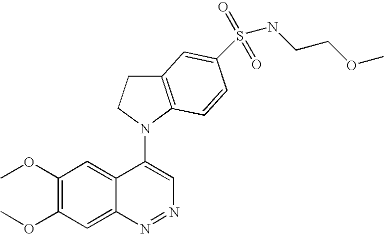 |
| 56) | 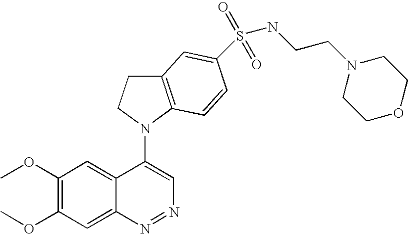 |
| 57) | 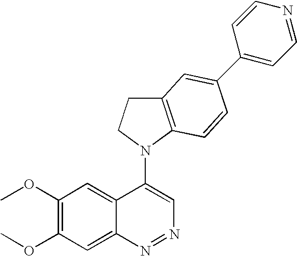 |
| 58) | 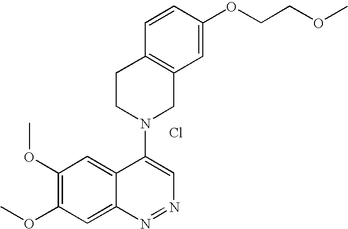 |
| 59) | 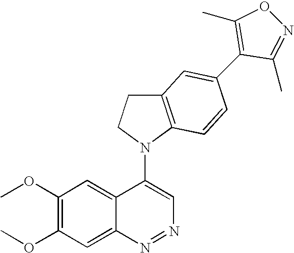 |
| 60) | 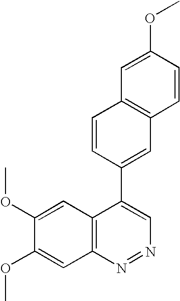 |
| 61) | 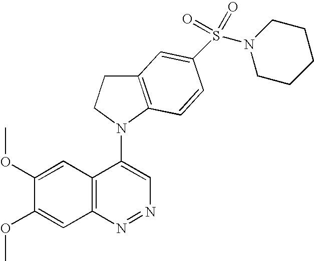 |
| 62) | 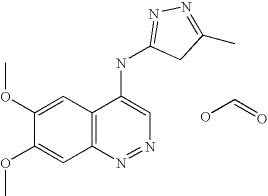 |
| 63) | 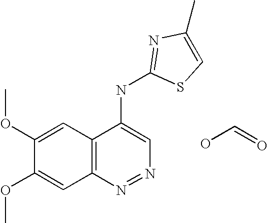 |
| 64) | 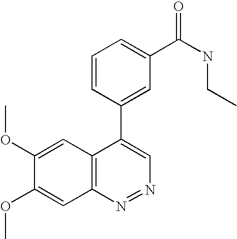 |
| 65) | 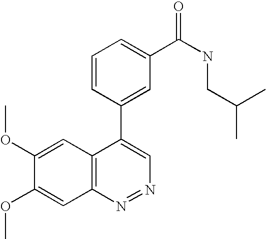 |
| 66) | 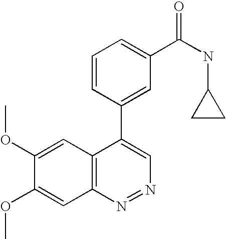 |
| 67) | 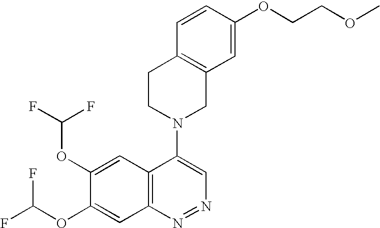 |
| 68) | 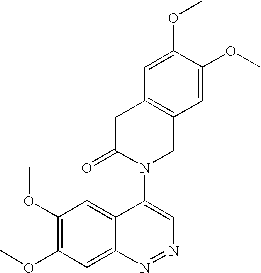 |
| 69) | 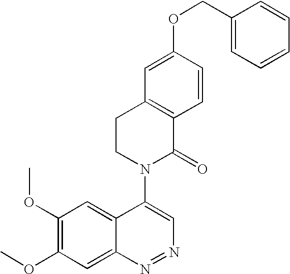 |
| 70) | 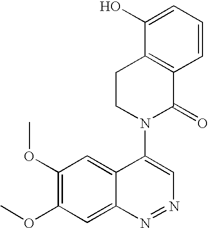 |
| 71) | 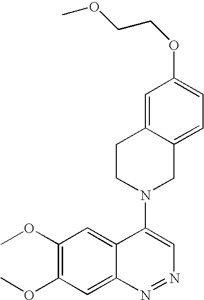 |
| 72) | 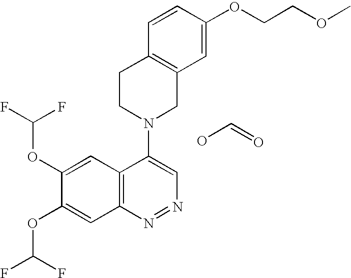 |
| 73) | 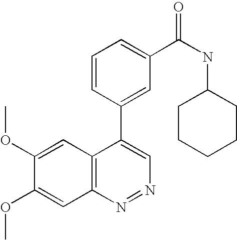 |
| 74) | 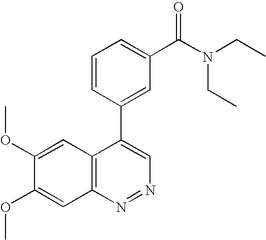 |
| 75) | 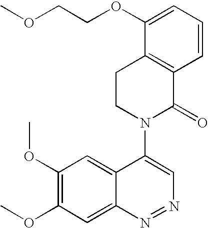 |
| 76) | 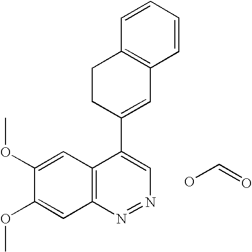 |
| 77) | 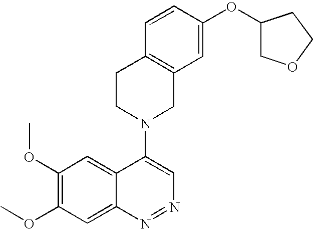 |
| 78) | 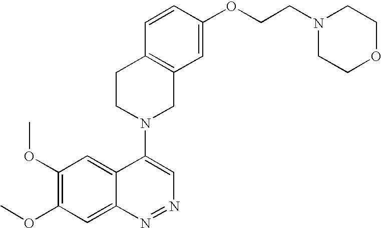 |
| 79) | 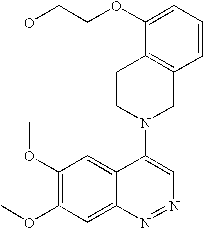 |
| 80) | 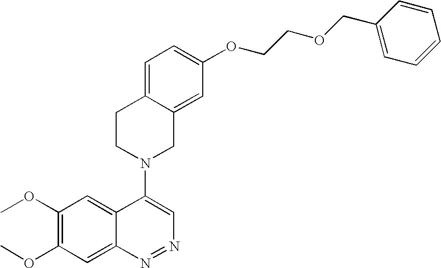 |
| 81) | 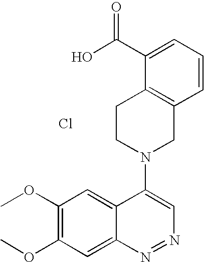 |
| 82) | 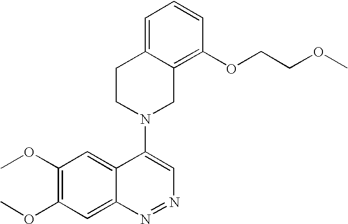 |

According to a further compound and/or method aspect of the invention, the compounds of the invention are selected from:
83) (4,5-Dihydro-4,4-dimethyl-1-isopropyl-1H-imidazol-2-yl) (6,7-dihydroxycinnolin-4-yl)-acetonitrile,
84) (1-Benzyl-1H-imidazol-2-yl)(6,7-dimethoxycinnolin-4-yl)acetonitrile,
85) (6,7-Dimethoxycinnolin-4-yl)(pyridin-3-yl)acetonitrile,
86) (6,7-Dimethoxycinnolin-4-yl)[2-(trifluoromethyl)phenyl]acetonitrile,
87) (4,5-Dihydro-1-isopropyl-1H-imidazol-2-yl) (6,7-dimethoxycinnolin-4-yl)acetonitrile,
88) 2-(6,7-Dimethoxycinnolin-4-yl)-1,2,3,4-tetrahydroisoquinoline-7-carbonitrile,
89) 4-(3,4-Dihydro-6-methoxy-isoquinolin-2(1H)-yl)-6,7-dimethoxycinnoline,
90) 4-(3,4-Dihydro-7-fluoro-isoquinolin-2(1H)-yl)-6,7-dimethoxycinnoline,
91) Methyl 2-(6,7-Dimethoxycinnolin-4-yl)-1,2,3,4-tetrahydroisoquinoline-5-carboxylate,
92) 4-(3,4-Dihydro-7-nitroisoquinolin-2(1H)-yl)-6,7-dimethoxycinnoline,
93) 4-(6,7-Diethoxy-3,4-dihydro-isoquinolin-2(1H)-yl)-6,7-dimethoxycinnoline,
94) 4-(3,4-Dihydro-5-nitroisoquinolin-2(1H)-yl)-6,7-dimethoxycinnoline,
95) 4-(1,3-Dihydro-2H-isoindol-2-yl)-6,7-dimethoxycinnoline,
96) Methyl 2-(6,7-dimethoxycinnolin-4-yl)-1,2,3,4-tetrahydroisoquinoline-7-carboxylate,
97) 4-[4-(3-Chlorophenyl)piperazin-1-yl]-6,7-dimethoxycinnoline,
98) 4-(3,4-Dihydroisoquinolin-2(1H)-yl)-6,7-dimethoxycinnoline,
99) (6,7-Dimethoxy-cinnolin-4-yl)-(4,5-dihydro-(4S)-4-phenyl-oxazol-2-yl)acetonitrile,
100) 4-(3,4-Dihydro-6,7-dimethoxy-isoquinolin-2(1H)-yl)-6,7-dimethoxycinnoline,
101) 4-(3,4-Dihydroquinolin-1(2H)-yl)-6,7-dimethoxycinnoline,
102) 6,7-Dimethoxy-(4-morpholin-4-yl)cinnoline,
103) 4-[4-(1,2-Benzisothiazol-3-yl)piperazin-1-yl]-6,7-dimethoxycinnoline,
104) 6,7-Dimethoxy-4-[(4aR, 8aS)-octahydroisoquinolin-2(1H)-yl]cinnoline,
105) 4-{4-[Bis(4-fluorophenyl)methyl]piperazin-1-yl}-6,7-dimethoxycinnoline,
106) 6,7-Dimethoxy-4-piperidin-1-ylcinnoline,
107) 4-[4-(1,3-Benzodioxol-5-ylmethyl)piperazin-1-yl]-6,7-dimethoxycinnoline,
108) 6-(6,7-Dimethoxycinnolin-4-yl)-5,6,7,8-tetrahydro-[1,3]-dioxolo[4,5-g]isoquinoline, and
109) (1-Benzyl-4,5-dihydro-1H-imidazol-2-yl)(6,7-dimethoxycinnolin-4-yl)acetonitrile,

wherein salts listed above can also be in free base form or in the form of another pharmaceutically acceptable salt, and free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof ) can also be in the form of a polymorph, and

wherein if the compound exhibits chirality it can be in the form of a mixture of enantiomers such as a racemate or a mixture of diastereomers, or can be in the form of a single enantiomer or a single diastereomer.

Intermediate compounds used in synthesizing the compounds of Formulas I and II include:
(4,5-Dihydro-4,4-dimethyl-1-isopropyl-1H-imidazol-2-yl)acetonitrile,
4-Bromo-6,7-dimethoxycinnoline,
6,7-Dimethoxycinnolin-4-ol,
(4,5-Dihydro-1-isopropyl-1H-imidazol-2-yl)acetonitrile,
[4,5-Dihydro-(4S)-4-phenyl-1,3-oxazol-2-yl]acetonitrile,
4-Chloro-6,7-dimethoxycinnoline, and
(1-Benzyl-4,5-dihydro-1H-imidazol-2-yl)acetonitrile.

According to a further method aspect of the invention, the compounds to be administered to the patient are selected from:
83) (4,5-Dihydro-4,4-dimethyl-1-isopropyl-1H-imidazol-2-yl) (6,7-dihydroxycinnolin-4-yl)-acetonitrile,
84) (1-Benzyl-1H-imidazol-2-yl)(6,7-dimethoxycinnolin-4-yl)acetonitrile,
85) (6,7-Dimethoxycinnolin-4-yl)(pyridin-3-yl)acetonitrile,
86) (6,7-Dimethoxycinnolin-4-yl)[2-(trifluoromethyl)phenyl]acetonitrile,
87) (4,5-Dihydro-1-isopropyl-1H-imidazol-2-yl) (6,7-dimethoxycinnolin-4-yl)acetonitrile,
88) 2-(6,7-Dimethoxycinnolin-4-yl)-1,2,3,4-tetrahydroisoquinoline-7-carbonitrile,
89) 4-(3,4-Dihydro-6-methoxy-isoquinolin-2(1H)-yl)-6,7-dimethoxycinnoline,
90) 4-(3,4-Dihydro-7-fluoro-isoquinolin-2(1H)-yl)-6,7-dimethoxycinnoline,
91) Methyl 2-(6,7-Dimethoxycinnolin-4-yl)-1,2,3,4-tetrahydroisoquinoline-5-carboxylate,
92) 4-(3,4-Dihydro-7-nitroisoquinolin-2(1H)-yl)-6,7-dimethoxycinnoline,
93) 4-(6,7-Diethoxy-3,4-dihydro-isoquinolin-2(1H)-yl)-6,7-dimethoxycinnoline,
94) 4-(3,4-Dihydro-5-nitroisoquinolin-2(1H)-yl)-6,7-dimethoxycinnoline,
95) 4-(1,3-Dihydro-2H-isoindol-2-yl)-6,7-dimethoxycinnoline,
96) Methyl 2-(6,7-dimethoxycinnolin-4-yl)-1,2,3,4-tetrahydroisoquinoline-7-carboxylate,
97) 4-[4-(3-Chlorophenyl)piperazin-1-yl]-6,7-dimethoxycinnoline,
98) 4-(3,4-Dihydroisoquinolin-2(1H)-yl)-6,7-dimethoxycinnoline,
99) (6,7-Dimethoxy-cinnolin-4-yl)-(4,5-dihydro-(4S)-4-phenyl-oxazol-2-yl)acetonitrile,
100) 4-(3,4-Dihydro-6,7-dimethoxy-isoquinolin-2(1H)-yl)-6,7-dimethoxycinnoline,
101) 4-(3,4-Dihydroquinolin-1(2H)-yl)-6,7-dimethoxycinnoline,
102) 6,7-Dimethoxy-(4-morpholin-4-yl)cinnoline,
103) 4-[4-(1,2-Benzisothiazol-3-yl)piperazin-1-yl]-6,7-dimethoxycinnoline,
104) 6,7-Dimethoxy-4-[(4aR,8aS)-octahydroisoquinolin-2(1H)-yl]cinnoline,
105) 4-{4-[Bis(4-fluorophenyl)methyl]piperazin-1-yl}-6,7-dimethoxycinnoline,
106) 6,7-Dimethoxy-4-piperidin-1-ylcinnoline,
107) 4-[4-(1,3-Benzodioxol-5-ylmethyl)piperazin-1-yl]-6,7-dimethoxycinnoline,
108) 6-(6,7-Dimethoxycinnolin-4-yl)-5,6,7,8-tetrahydro-[1,3]-dioxolo[4,5-g]isoquinoline,
109) (1-Benzyl-4,5-dihydro-1H-imidazol-2-yl)(6,7-dimethoxycinnolin-4-yl)acetonitrile,
110) (4,5-Dihydro-4,4-dimethyl-1-isopropyl-1H-imidazol-2-yl) (6,7-dimethoxycinnolin-4-yl)-acetonitrile hydrochloride, and
111) (4,5-Dihydro-4,4-dimethyl-1-isopropyl-1H-imidazol-2-yl) (6,7-dimethoxycinnolin-4-yl)-acetonitrile,

wherein salts listed above can also be in free base form or in the form of another pharmaceutically acceptable salt, and free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

The following table presents further structures for selected compounds of Formulas I and II in accordance with the present invention:

| Compound | Structure |
|---|---|
| 83) | 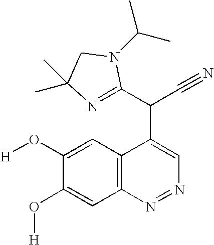 |
| 84) | 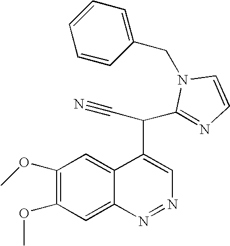 |
| 85) | 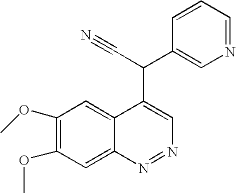 |
| 86) | 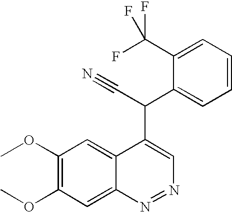 |
| 87) | 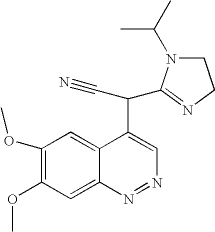 |
| 88) | 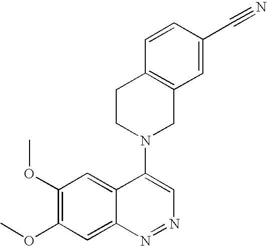 |
| 89) | 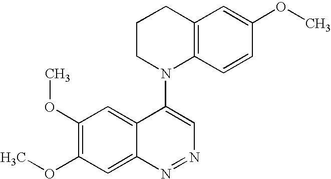 |
| 90) | 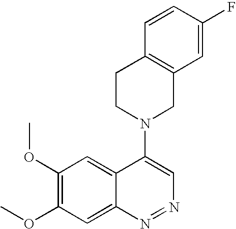 |
| 91) | 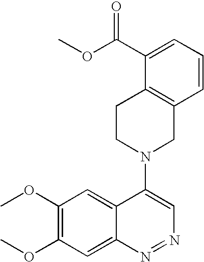 |
| 92) | 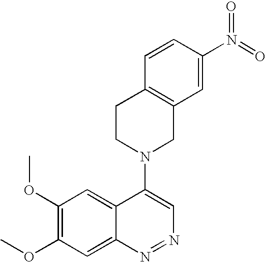 |
| 93) | 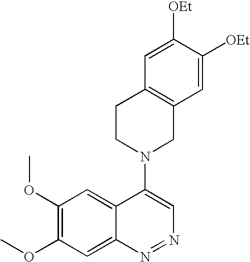 |
| 94) | 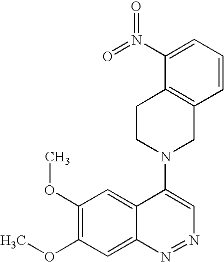 |
| 95) | 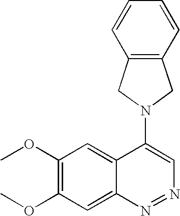 |
| 96) | 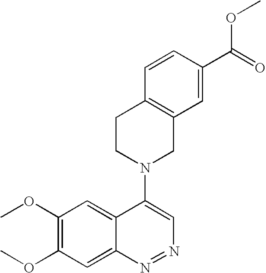 |
| 97) | 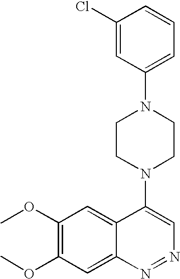 |
| 98) | 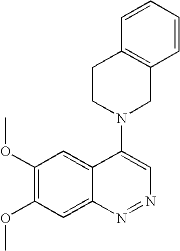 |
| 99) | 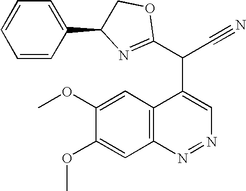 |
| 100) | 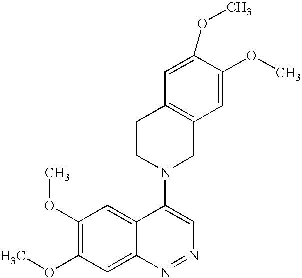 |
| 101) | 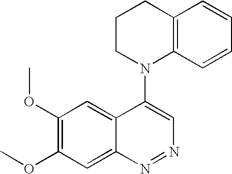 |
| 102) | 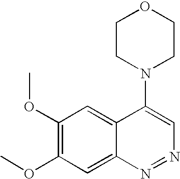 |
| 103) | 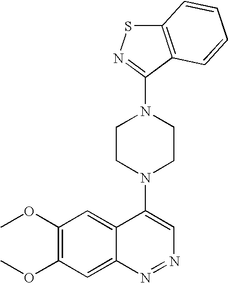 |
| 104) | 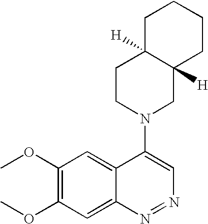 |
| 105) | 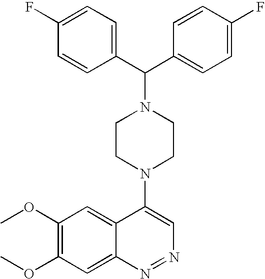 |
| 106) | 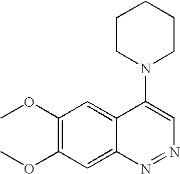 |
| 107) | 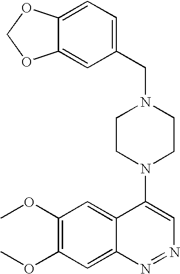 |
| 108) | 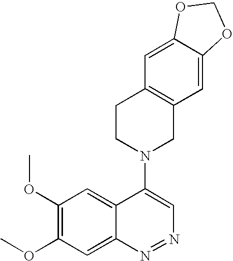 |
| 109) | 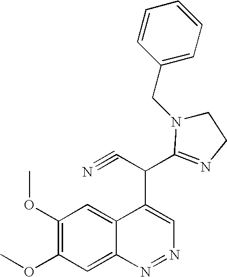 |
| 110) | 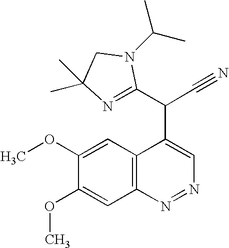 |
| 111) | 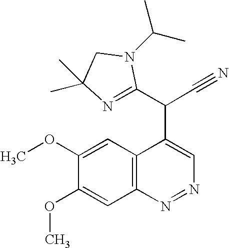 |

Additional aspects of the present invention include pharmaceutical compositions comprising a compound of this invention and a pharmaceutically acceptable carrier and, optionally, one or more additional active agent(s) as discussed below. A further preferred aspect includes a method of inhibiting a PDE10 enzyme, e.g., as determined by a conventional assay or one described herein, either in vitro or in vivo (in an animal, e.g., in an animal model, or in a mammal or in a human); a method of treating a psychiatric or neurological syndrome, e.g., psychoses, obsessive-compulsive disorder and/or Parkinson's disease; a method of treating a disease state modulated by PDE10 activity, in a patient, such as a mammal, e.g., a human, e.g., those disease states mentioned herein. Additionally, the invention includes methods of treating diseases affecting the function of the basal ganglia such as schizophrenia and obsessive-compulsive disorder.

Methods of the invention include, but are not limited to, methods of enhancing cognition in a patient in whom such enhancement is desired, methods of treating a patient suffering from cognition impairment or decline, methods of treating a patient having a disease involving decreased cAMP and/or cGMP levels, methods of inhibiting PDE10 enzyme activity in a patient, methods of treating a patient suffering psychoses, in particular schizophrenia or bipolar disorder, methods of treating a patient suffering from obsessive-compulsive disorder, methods of treating a patient suffering from Parkinson's disease. All methods comprise administering to the patient in need of such treatment an effective amount of one or more compounds of the invention.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The compounds of the present invention may be prepared conventionally. Some of the known processes that can be used are described below. All starting materials are known or can be conventionally prepared from known starting materials.

The core heterocyclic entity of each of the drug candidates described is a 6,7-disubstituted cinnoline. These molecules have been prepared by several effective methods. One method involves cyclization of 2-alkynylanilines. Upon diazotization of the aniline, cyclization occurs onto the terminus of the alkyne, producing the cinnoline. Sonogashira couplings of alkynes to 2-iodoanilines produce the alkynylaniline starting materials. [Queguiner, G. et al. Tetrahedron, 2000, 56, 5499.]

Alternatively, 4-halocinnoline starting materials can be prepared by the method shown in scheme 1. Treatment of 2-amino-4,5-dialkoxyacetophenones 5 with sodium nitrite in concentrated HCl and water provides diazo compound intermediates that cyclize under heating to provide 6,7-dialkoxy-4-hydroxycinnolines 6. The desired 4-halocinnolines 7 are accessed by treatment of the hydroxycinnoline 6 with either phosphorous oxychloride or phosphorous oxybromide.

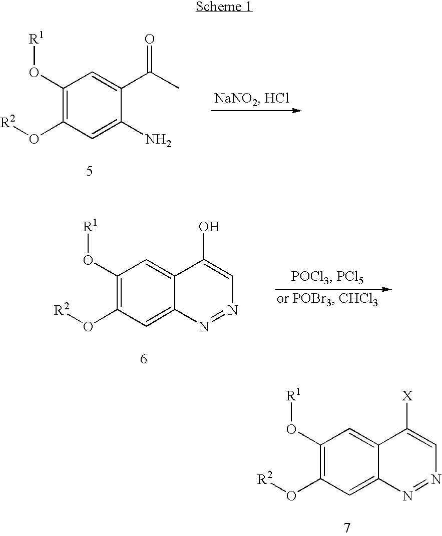

The chloride is formed by reaction of hydroxycinnoline 6 with neat phosphorous oxychloride, followed by recrystallization of the product after neutralization. [Castle, Raymond N. et al. J. Org. Chem., 1952,17,1571. ] The bromide is prepared by mixing a concentrated suspension of the hydroxycinnoline in chloroform and phosphorous oxybromide at room temperature and then warming to reflux for 8 to 16 hours. Extractive workup after neutralization and subsequent recrystallization from ethanol provides pure 4-bromocinnoline targets 7.

Dialkylated aminoacetophenones 5 are either commercially available (e.g., 2-amino-4,5-dimethoxyacetophenone) or can be synthesized by methods common to the art. Simple dialkyl ethers, wherein the alkyl groups at the 3,4-postions are the same, can be readily accessed by standard etherification reactions. For example, 3,4-dihydroxyacetophenone can be treated with an excess of cesium carbonate and the desired alkyl halide to directly provide the dialkylated product. Other bases such as triethylamine, sodium hydride, potassium carbonate, potassium hydride, etc. can be employed in combination with a variety of solvents, including acetone, acetonitrile, DMF, and THF.

Syntheses of differentially substituted 3,4-dialkyl ethers of 5 can be accomplished under standard conditions. If the desired substituent at the 3-position is the methyl ether, acetovanillone (3-methoxy-4-hydroxyacetophenone) can be utilized as a starting material. Simple etherification, as described above, can be utilized to provide the required 4-substitution. When etherification by alkylation proves difficult, recourse to Mitsunobu conditions often provides the desired products. This can generally be accomplished by treatment of the phenol with diethyl or diisopropyl azo-dicarboxylates, triphenylphosphine, and the desired alkyl alcohol in THF solution. Treatment of the phenol with chlorodifluoroacetic acid under basic conditions allows access to difluoromethyl ethers.

When ethers other than methyl are required at the 3-position, 3,4-dihydroxyacetophenone 1 can again be utilized as the starting material. 3,4-Dihydroxyacetophenone 1 can be selectively protected as its 4-benzyl ether 2 [Greenspan, Paul D. et al. J. Med. Chem., 1999, 42, 164.] by treatment with benzyl bromide and lithium carbonate in DMF solution (scheme 2). Functionalization of the remaining phenol with the desired alkyl halide to generate the fully substituted acetophenone 3 can be accomplished by any of etherification reactions described above, including Mitsunobu reaction. Removal of the benzyl ether by hydrogenolysis with palladium on carbon in alcoholic solvents such as methanol and a final etherification yields the 3,4-dialkoxyacetophenones 4. 2-Amino-4,5-dialkoxyacetophenones 5 may be prepared by nitration with nitric acid in one of several solvents including acetic acid or sulfuric acid at ice bath temperatures to provide 2-nitro-4,5-dialkoxyacetophenones [Iwamura, Michiko, et al., Bioorg. Med. Chem., 2002, 10, 675.]. The nitro group is then reduced by one of several methods, including hydrogenation with palladium on carbon, iron powder in acetic acid, or nickel boride, among others, to provide target anilines 5. [Castle, Raymond N. et al. J. Org. Chem. 1954, 19, 1117.]

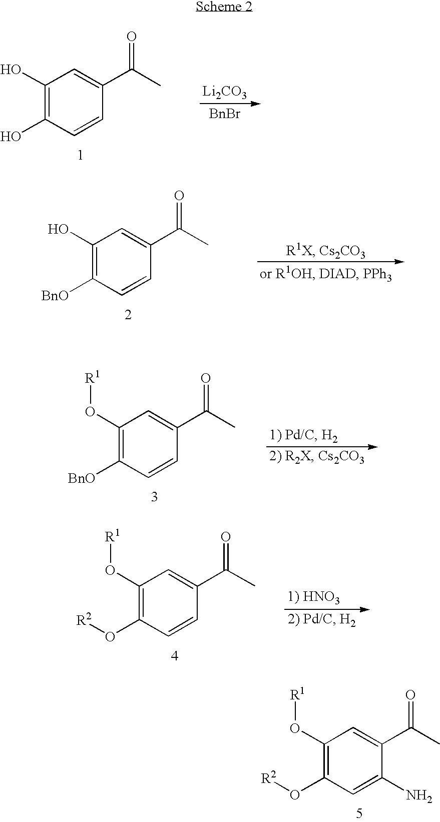

For many of the differentially substituted dialkoxy ethers, other starting materials can prove useful. A large number of substituted 3,4-dialkoxybenzaldehydes are commercially available and can readily be manipulated to provide the corresponding acetophenones. Simple addition of a methyl nucleophile such as methyl Grignard reagent or methyl lithium to the aldehyde in ether or THF solution at low temperature provides secondary benzylic alcohols. The alcohols, in turn, are readily oxidized to acetophenones 4 using a vast array of available methods, such as Dess-Martin periodinane, Swern oxidation, PCC, NaOCl, and the like. 4-Bromo-6,7-bis-difluoromethoxycinnoline analogs (Scheme 3) can be prepared from 2-acetylaniline derivative 12 as described above. Aniline 12 in turn may be synthesized from 3,4-dimethoxyacetophenone 8 by reaction with nitric acid to yield nitro intermediate 9. Cleavage of the methoxy groups by heating with pyridine-HCl provides catechol 10. Reaction with chlorodifluoroacetic acid provides bis-diflouromethoxy derivative 11 which undergoes reduction with Pd/C and hydrogen to give 12.

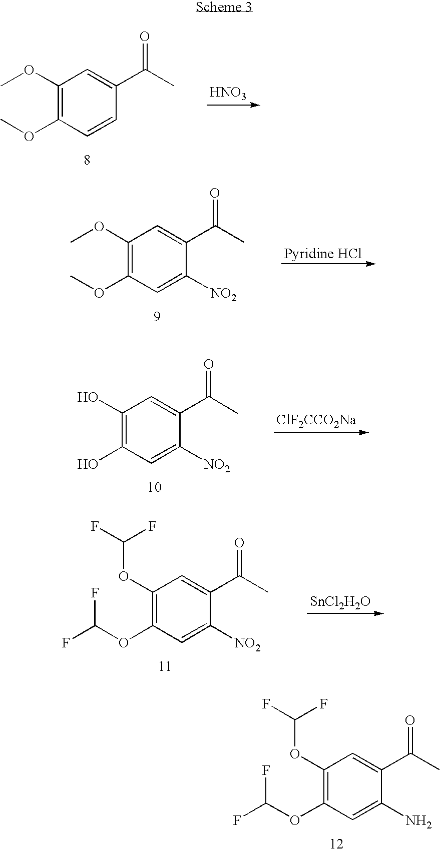

In order to access the compounds of the present invention, 4-halocinnolines 7 are coupled to a variety of different side chains. Simple amines, such as isopropyl, benzyl, or cyclopropylmethylamine can be heated directly, either conventionally or in the microwave, with the halocinnolines to produce the 4-aminocinnolines 13. [Lowrie, Harman S. J. Med. Chem., 1966, 9, 670.] In cases where the use of the amine as a solvent is cost prohibitive, the reaction can be promoted with copper salts such as copper iodide or copper acetate, or even with copper metal, in high boiling solvents such as DMSO. [Lowrie, Harman S. J. Med. Chem., 1966, 9, 670.]

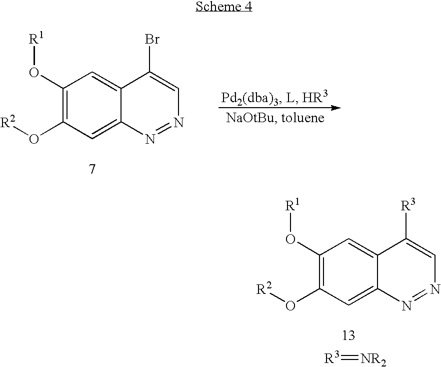

Alternatively, palladium mediated coupling of the bromocinnoline 7 with amines provides the desired 4-aminocinnolines 13 (Scheme 4). A large variety of conditions are effective in these reactions. Palladium sources include, for example, Pd(PPh₃)₄, Pd₂(dba)₃, Pd(OAc)₂, and others, while solvents such as toluene, DMF, THF, and acetonitrile may be employed. Bases and ligands have also been explored extensively, and may include, for example, NaOtBu, NaHMDS, NaOMe, Cs₂CO₃, and other bases. Ligands which may be employed include, but are not limited to, dppb, XANPHOS, BINAP, tBu₃P, and 2-dicyclohexylphosphino-2′-(N,N-dimethylamino)biphenyl. Optimal reaction conditions vary depending on the amine substrate used and also on the halocinnoline starting material. In the example shown in Scheme 4, Pd₂(dba)₃ is the preferred palladium source, with XANPHOS as the ligand and sodium t-butoxide as the base in toluene solution. To complete the couplings, the reactions are generally heated to between about 50 and about 100° C. for about 18 hours. Microwave heating may also be effective in many cases. When benzophenone imine is utilized as the amine source, the result is a protected form of 4-aminocinnoline 13. Upon hydrolysis of the imine, simple alkylations or reductive aminations of the amine can provide a variety of disubstituted alkylaminocinnolines. R³-Zinc halide reagents may also be employed.

In addition 4-bromocinnolines undergo Suzuki coupling reactions (scheme 5) to yield 4-arylcinnoline compounds 13.

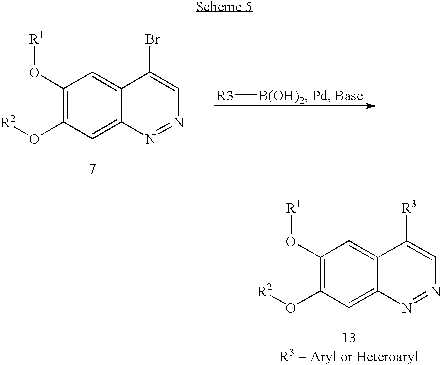

Alternatively, carbon nucleophiles generated by treatment of an activated alkyl with base can be coupled to halo-cinnolines 7 by nucleophilic displacement (scheme 6). Generally, these reactions can be accomplished if one of the substituents (R¹⁷ or R¹⁸) is aromatic or otherwise resonance withdrawing to provide stabilization to the developing anion. A variety of different conditions can be employed. Typically a strong base such as KHMDS, NaNH₂, or LDA, is utilized to deprotonate the side chain substrate at temperatures from about −78° C. to about 0° C. The halocinnoline is then added to the anion as a solution in solvents such as THF, DMF, or benzene, and the reactions are generally warmed to room temperature until complete.

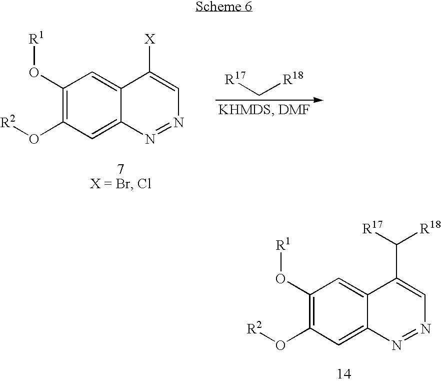

The formation of imidazoline heterocycles 18 requires the generation of a variety of substituted diamines 17 to be synthesized. Thus, resin supported chloroacetamides are reacted with amines, followed by amide reduction and then cleavage from the resin to provide appropriately substituted diamines 17. A combinatorial approach is effective [Barry, Clifton E. et al. J. Comb. Chem., 2003, 5, 172.]

Prerequisite diamines 17 and correspondingly, the cyano-imidazolines 18 can be prepared from nitro alcohols 15 as outlined in scheme 7 [Senkus, Murray et al. J. Am. Chem. Soc. 1946, 68, 10].

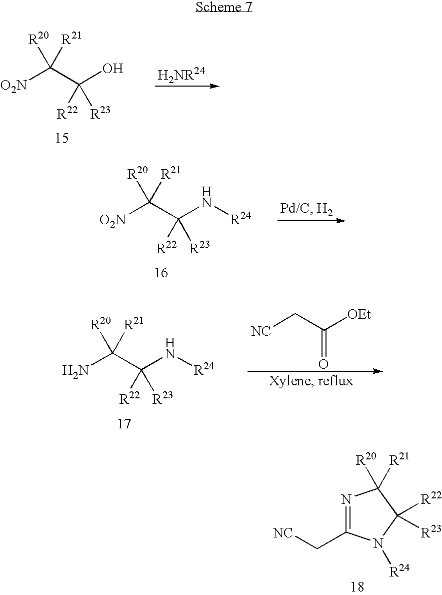

Thus, heating substituted nitro ethanols 15 with primary amines provides nitro ethylamines 16 (condensation reaction). Reduction of the nitro group to the corresponding amine by hydrogenation over palladium on carbon or with iron powder provides the precursor diamine 17. Condensation with ethyl cyanoacetate provides the desired cyanoimidazolines 18. [Riebsomer, J. L. et al., J. Org. Chem., 1950, 15, 909.]

An alternative approach to the desired cyanoimidazolines 18 involves cyclization of diamines 17 with cyano-imidate 19 (scheme 8). [Meyers, A. I. et al., Tetrahedron, 2002, 58, 207.] Treatment of the imidate 19 with amino alcohols or amino thiols provides oxazoline and thiozoline heterocycles 21.

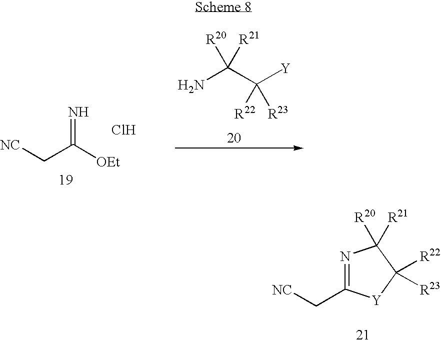

Various carboxylate derivatives can be obtained from the cyano-heterocycle side chains appended to cinnoline 14. Reductions of the nitrile provide amines, which can be further manipulated; while hydrolysis of the nitrile provides carboxamides, and carboxylic acids.

Several methods are available for the synthesis of variously substituted tetrahydroisoquinoline (THIQ) compounds. For example, commercially available THIQ 22 can be protected as the 1-amido analog 23 by reaction with acetic anhydride or acetyl chloride and base (scheme 9). Cleavage of the methoxy group with BBr₃ provides phenolic intermediate 24, which undergoes alkylation reactions with various alkyl halides (such as methoxyethyl chloride) to generate 1-amido analogs 25, which can be hydrolyzed under basic conditions to yield target THIQ compounds 26.

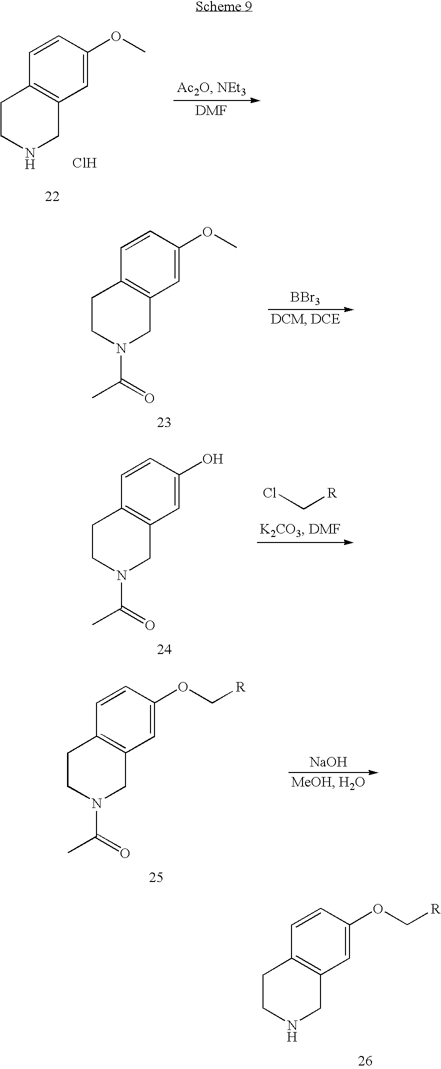

Alternatively, THIQ compounds can be synthesized from phenethylamines 27 by reaction with ethyl chloroformate to generate carbamates of the type 28. Acid promoted cyclization yields dihydroquinolones 29 which are reduced to the target THIQ compound by reaction with lithium aluminum hydride (LAH) (scheme 10).

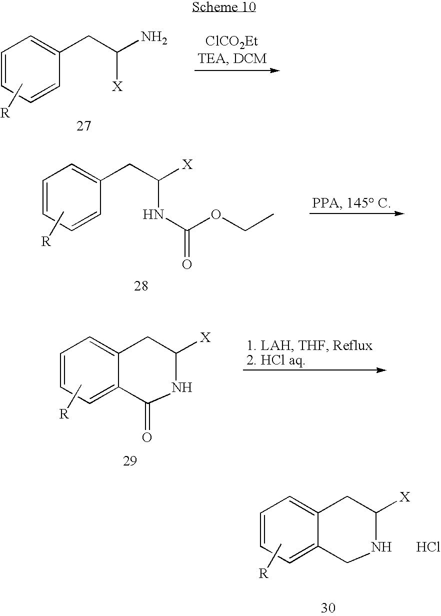

The THIQ compounds can be further functionalized by generating phenol 32 from methoxy derivative 31 by reaction with BBr₃ followed by alkylation type reactions. Thus, as exemplified in scheme 11, dihydroisoquinolone 32 undergoes reaction with alkyl halides, for example 1-chloro-2-methoxyethane, in the presence of a base like K₂CO₃ and a phase transfer catalyst to provide alkyloxy intermediate 33. Subsequent reduction of the amide with borane provides target 26.

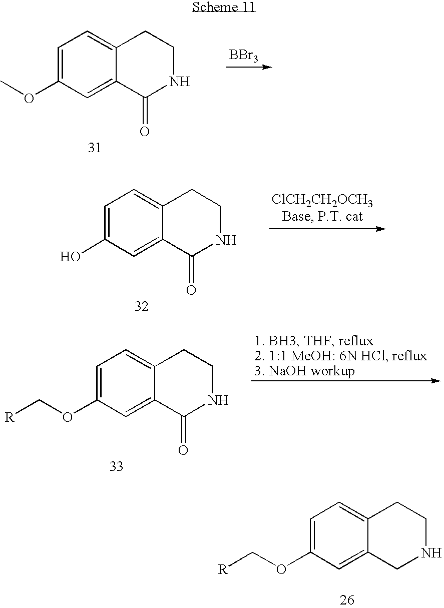

Further, the phenol derivatives 34 can undergo arylation and heteroarylation reactions with appropriately substituted boronic acids to yield dihydroisoquinilones of the type 35 (scheme 12). Reduction with LAH produces THIQ targets 36.

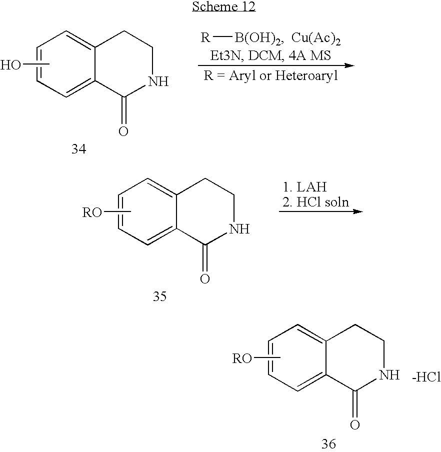

Furthermore, phenols 34 can be converted to the corresponding triflates which undergo reaction with aryl and heteroaryl boronic acids to yield aryl and heteroaryl substituted tetrahydroisoquinolines 39 after treatment with LAH (scheme 13). Additionally, it is possible to displace the triflate with a variety amines under Buchwald conditions.

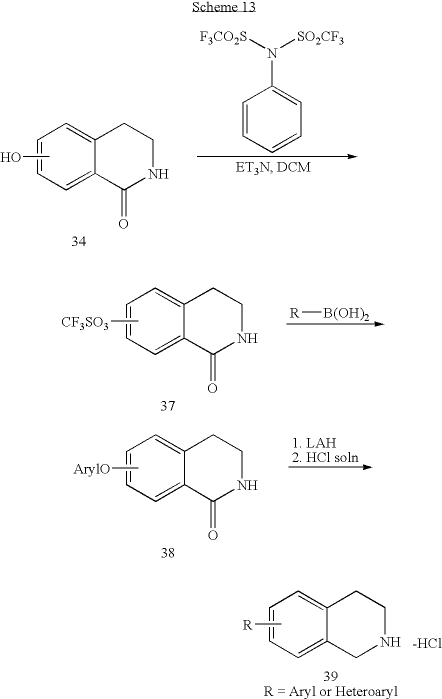

Nitration of dihydroisoquinolones of the type 40 by reaction with nitric acid and sulfuric acid produces 7-nitrodihydroisoquinolones 41 (scheme 14). Borane reduction to 7-nitrotetrahydroisoquinoline 42 followed by acetylation with trifluoroacetic anhydride provides protected nitro analog 43. Reductive hydrogenation over palladium on carbon and subsequent acetylation with acetic anhydride generates acetamide 44. Trifluoroacetamnide hydrolysis by reaction with potassium carbonate in methanol produces tetrahydroisoquinoline 45.

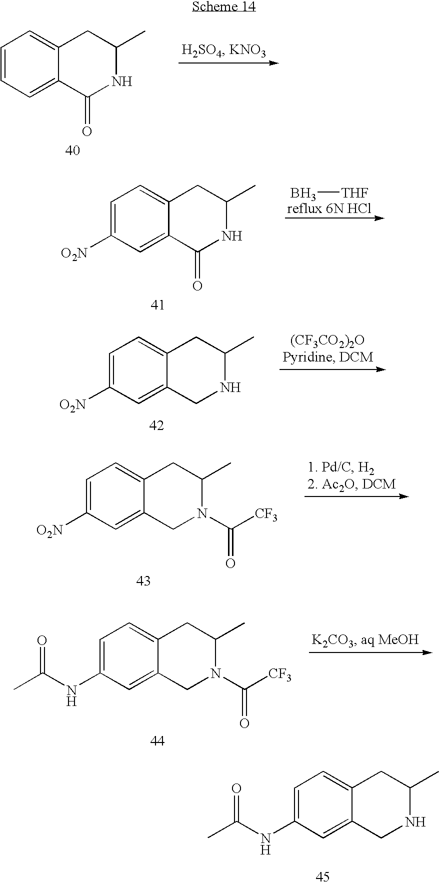

Aminosulfonyl substituted tetrahydroquinolines 49 can be synthesized from N-acetyltetrahydroquinoline 46 (scheme 15). Thus, treatment of 46 with chlorosulfonic acid provides 6-chlorosulfonyl derivative 47. Reaction with an amine, for example dimethylamine, and subsequent acid induced hydrolysis of the acetamide provides target 49.

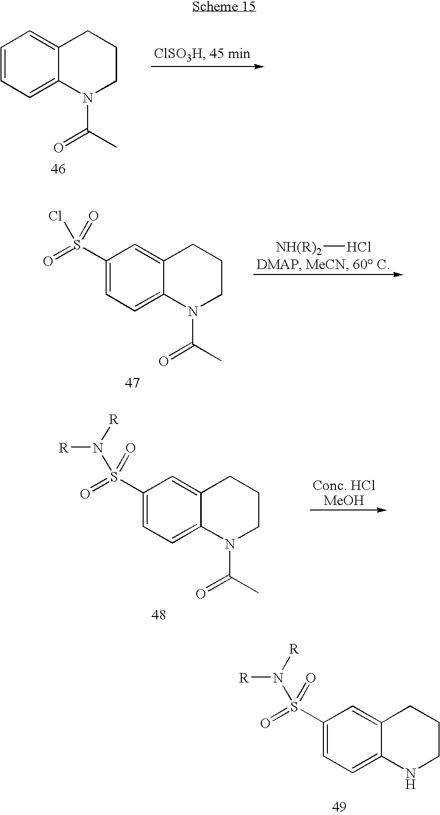

Dihydroquinolones 52 and tetrahydroquinolines such as 53 can be prepared as described in scheme 16. Thus, diazatization and then reaction with sulfur dioxide and cuprous chloride provides sulfonyl chloride derivative 51. Reaction with amines such as dimethylamine provides sulfonamide dihydroquinolones 52, which is readily reduced by reaction with borane in THF to generate the corresponding tetrahydroquinolines 53.

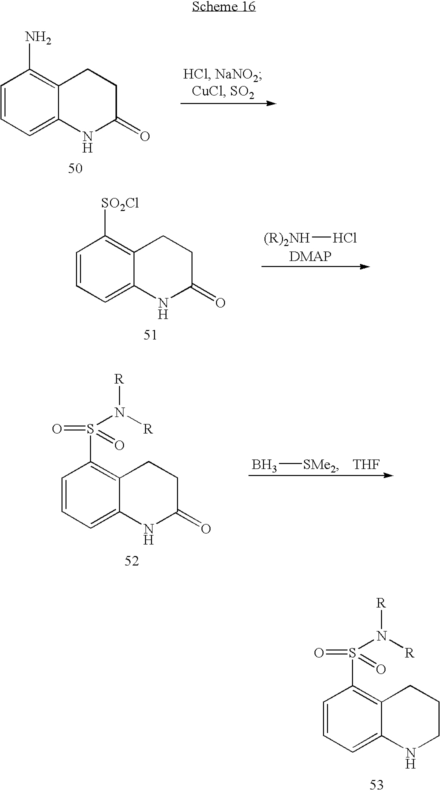

Amino-dihydroquinolone 50 undergoes reaction with methanesulfonyl chloride to yield N,N-dimethanesulfonylamino derivative 54 (scheme 17). Reduction of the dihydroquinolone to the tetrahydroquinoline with borane and subsequent treatment with lithium hydroxide yields 5-methylsulfonamido-tetrahydroquinolines 56.

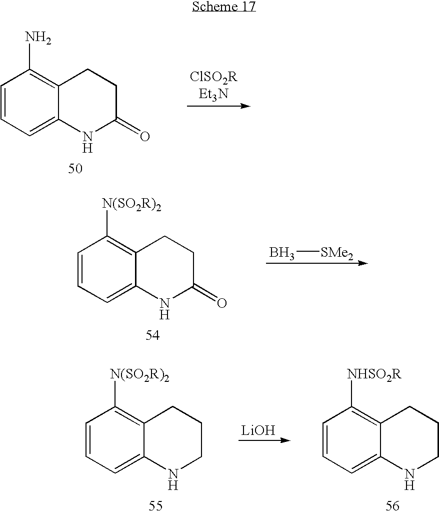

Aminosulfonyl indoline compounds (scheme 18) can be prepared in a similar manner as described in scheme 16. Thus, N-acetyl 5-chlorosulfonylindolines 57 undergo reactions with amines to generate aminosulfonylindolines 59, after N-acetyl hydrolysis of 58 using sodium hydroxide.

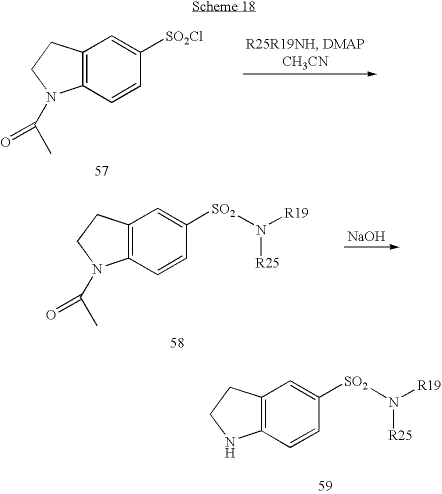

One of ordinary skill in the art will recognize that some of the compounds of Formulas I, Ia, II, Ia and III can exist in different geometrical isomeric forms. In addition, some of the compounds of the present invention possess one or more asymmetric atoms and are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomeric mixtures, racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereomeric salts using an optically active acid or base or formation of covalent diastereomers.

Examples of appropriate acids include, but are not limited to, tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel O D and Chiracel O J among many others, all routinely selectable. Enzymatic separations, with or without derivitization, are also useful. The optically active compounds of Formulas I, Ia, II, Ia and III can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp. CAN 133:68895 AN 2000:473538 CAPLUS; Kabalka, George W.; Varma, Rajender S. The Synthesis of Radiolabeled Compounds via Organometallic Intermediates, Tetrahedron, 1989, 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem., 1981, 64(1-2), 9-32. CODEN: JRACBN ISSN:0022-4081, CAN 95:76229 AN 1981:476229 CAPLUS.

The present invention also relates to useful forms of the compounds as disclosed herein, including free base forms, as well as pharmaceutically acceptable salts or prodrugs of all the compounds of the present invention for which salts or prodrugs can be prepared. Pharmaceutically acceptable salts include those obtained by reacting the main compound, functioning as a base, with an inorganic or organic acid to form a salt, for example, but not limited to, salts of hydrochloric acid, sulfuric acid, phosphoric acid, hydrobromic, methanesulfonic acid, camphorsulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts may be prepared by reacting the compounds of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharamaceutically acceptable salt can be a hydrochloride, a hydroformate, hydrobromide, or a maleate.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formulas I, Ia, II, Ia and III can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formulas I, Ia, II, Ia and III can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates.

The compounds of the invention can be administered alone or as an active ingredient of a formulation. Thus, the present invention also includes pharmaceutical compositions of one or more compounds of Formulas I, Ia, II, Ia and/or III containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

In view of their high degree of selective PDE10 inhibition, the compounds of the present invention can be administered to anyone requiring PDE10 inhibition. Administration may be accomplished according to patient needs, for example, orally, nasally, parenterally (subcutaneously, intravenously, intramuscularly, intrastemally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the invention, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The compounds can be administered as the sole active agent or in combination with other pharmaceutical agents such as other agents used in the treatment of psychoses, especially schizophrenia and bipolar disorder, obsessive-compulsive disorder, Parkinson's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range.

The compounds can be administered in combination with other pharmaceutical agents used in the treatment of schizophrenia, e.g., Clozaril, Zyprexa, Risperidone, and Seroquel. Thus, the invention also includes methods for treating schizophrenia, including memory impairment associated with schizophrenia, comprising administering to a patient, simultaneously or sequentially, the compound of the invention and one or more additional agents used in the treatment of schizophrenia such as, but not limited to, Clozaril, Zyprexa, Risperidone, and Seroquel. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of schizophrenia, e.g., Clozaril, Zyprexa, Risperidone, and Seroquel. Similarly, the invention also includes kits containing a composition comprising a compound according to Formula I, Ia, II, Ia and/or III and another composition comprising one or more additional pharmaceutical agents used in the treatment of schizophrenia, e.g., Clozaril, Zyprexa, Risperidone, and Seroquel.

In addition, the compounds can be administered in combination with other pharmaceutical agents used in the treatment bipolar disorder such as Lithium, Zyprexa, and Depakote. Thus, the invention also includes methods for treating bipolar disorder, including treating memory and/or cognitive impairment associated with the disease, comprising administering to a patient, simultaneously or sequentially, the compound of the invention and one or more additional agents used in the treatment of bipolar disorder such as, but not limited to, Lithium, Zyprexa, and Depakote. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of bipolar disorder such as, but not limited to, Lithium, Zyprexa, and Depakote. Similarly, the invention also includes kits containing a composition comprising a compound according to Formula I, Ia, II, Ia and/or III and another composition comprising one or more additional pharmaceutical agents used in the treatment of bipolar disorder such as Lithium, Zyprexa, and Depakote.

The invention also includes methods for treating Parkinson's disease, including treating memory and/or cognitive impairment associated with Parkinson's disease, comprising administering to a patient, simultaneously or sequentially, the compound of the invention and one or more additional agents used in the treatment of Parkinson's disease such as, but not limited to, Levodopa, Parlodel, Permax, Mirapex, Tasmar, Contan, Kemadin, Artane, and Cogentin. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of Parkinson's disease, such as, but not limited to, Levodopa, Parlodel, Permax, Mirapex, Tasmar, Contan, Kemadin, Artane, and Cogentin. Similarly, the invention also includes kits containing a composition comprising a compound according to Formula I, Ia, II, Iha and/or III and another composition comprising one or more additional pharmaceutical agents gent used in the treatment of Parkinson's disease such as, but not limited to, Levodopa, Parlodel, Permax, Mirapex, Tasmar, Contan, Kemadin, Artane, and Cogentin.

In addition, the invention includes methods for treating memory and/or cognitive impairment associated with Alzheimer's disease comprising administering to a patient, simultaneously or sequentially, the compound of the invention and one or more additional agents used in the treatment of Alzheimer's disease such as, but not limited to, Reminyl, Cognex, Aricept, Exelon, Akatinol, Neotropin, Eldepryl, Estrogen and Cliquinol. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of Alzheimer's disease such as, but not limited to, Reminyl, Cognex, Aricept, Exelon, Akatinol, Neotropin, Eldepryl, Estrogen and Cliquinol. Similarly, the invention also includes kits containing a composition comprising a compound according to Formula I, Ia, II, IIa and/or III and another composition comprising one or more additional pharmaceutical agents used in the treatment of Alzheimer's disease such as, but not limited to Reminyl, Cognex, Aricept, Exelon, Akatinol, Neotropin, Eldepryl, Estrogen and Cliquinol.

Another aspect of the invention includes methods for treating memory and/or cognitive impairment associated with dementia comprising administering to a patient, simultaneously or sequentially, the compound of the invention and one or more additional agents used in the treatment of dementia such as, but not limited to, Thioridazine, Haloperidol, Risperidone, Cognex, Aricept, and Exelon. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of dementia such as, but not limited to, Thioridazine, Haloperidol, Risperidone, Cognex, Aricept, and Exelon. Similarly, the invention also includes kits containing a composition comprising a compound according to Formula I, Ia, II, Iha and/or III and another composition comprising one or more additional pharmaceutical agents used in the treatment of dementia such as, but not limited to, Thioridazine, Haloperidol, Risperidone, Cognex, Aricept, and Exelon.

A further aspect of the invention includes methods for treating memory and/or cognitive impairment associated with epilepsy comprising administering to a patient, simultaneously or sequentially, the compound of the invention and one or more additional agents used in the treatment of epilepsy such as, but not limited to, Dilantin, Luminol, Tegretol, Depakote, Depakene, Zarontin, Neurontin, Barbita, Solfeton, and Felbatol. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol. Similarly, the invention also includes kits containing a composition comprising a compound according to Formula I, Ia, II, Ia and/or III and another composition comprising one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol.

A further aspect of the invention includes methods for treating memory and/or cognitive impairment associated with multiple sclerosis comprising administering to a patient, simultaneously or sequentially, the compound of the invention and one or more additional agents used in the treatment of multiple sclerosis such as, but not limited to, Detrol, Ditropan XL, OxyContin, Betaseron, Avonex, Azothioprine, Methotrexate, and Copaxone. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of multiple sclerosis such as, but not limited to, Detrol, Ditropan XL, OxyContin, Betaseron, Avonex, Azothioprine, Methotrexate, and Copaxone. Similarly, the invention also includes kits containing a composition comprising a compound according to Formula I, Ia, II, Ila and/or III and another composition comprising one or more additional pharmaceutical agents used in the treatment of multiple sclerosis such as, but not limited to, Detrol, Ditropan XL, OxyContin, Betaseron, Avonex, Azothioprine, Methotrexate, and Copaxone.

The invention further includes methods for treating Huntington's disease, including treating memory and/or cognitive impairment associated with Huntington's disease, comprising administering to a patient, simultaneously or sequentially, the compound of the invention and one or more additional agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. In methods using simultaneous administration, the agents can be present in a combined composition or can be administered separately. As a result, the invention also includes compositions comprising a compound according to Formula I, Ia, II, Ia and/or III and one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. Similarly, the invention also includes kits containing a composition comprising a compound according to Formula I, Ia, II, Ia and/or III and another composition comprising one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone.

The present invention involves compounds that inhibit PDE10 enzyme activity. PDE10 inhibitors will raise the levels of cAMP or cGMP within cells that express PDE10. Inhibition of PDE10 enzyme activity may be of relevance to diseases caused by deficient amounts of cAMP or cGMP in cells. Alternatively, PDE10 inhibitors may be of benefit in cases wherein raising the amount of cAMP or cGMP above normal levels results in a therapeutic effect. Inhibitors of PDE10 may be used to treat disorders of the peripheral and central nervous system, cardiovascular diseases, cancer, gastro-enterological diseases, endocrinological diseases and urological diseases. Thus, the present invention includes methods of selective inhibition of PDE10 enzymes in patients, e.g., mammals, especially humans, wherein such inhibition has a therapeutic effect, such as where such inhibition may relieve conditions involving neurological or psychiatric syndromes, such as the loss of memory or psychoses. Such methods comprise administering to a patient in need thereof, especially a mammal, most especially a human, an inhibitory amount of a compound of the invention, alone or as part of a formulation, as disclosed herein.

Indications that may be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsion disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The present invention includes methods for treating patients suffering from all forms of psychoses, including, but not limited to, schizophrenia, late-onset schizophrenia, schizoaffective disorders, prodromal schizophrenia, and bipolar disorders. Treatment may be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality may also be treated with PDE10 inhibitors.

Obsessive-compulsive disorder (OCD) has been linked to deficits in the frontal-striatal neuronal pathways. (Saxena S. et al., Br. J. Psychiatry SuppL., 1998; (35):26-37.) Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. PDE10 inhibitors should be useful for the indication of OCD. OCD may result, in some cases, from streptococcal infections that cause autoimmune reactions in the basal ganglia (Giedd J N et al., Am J Psychiatry., 2000 February; 157(2):281-3). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that since PDE10 degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The present invention includes methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. In another embodiment, the invention includes methods for dealing with memory loss resulting from the use of general anesthetics, chemotherapy, radiation treatment, post-surgical trauma, and therapeutic intervention. Thus, in accordance with a preferred embodiment, the present invention includes methods of treating patients suffering from memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases. The invention also relates to agents and/or methods to stimulate the formation of memory in “normal” subjects (i.e., subjects who do not exhibit an abnormal or pathological decrease in a memory finction), e.g., ageing middle-aged subjects.

The invention is also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-i (ataxin-1); spinocerebellar ataxia type-2 (ataxin-2); spinocerebellar ataxia type-3 also called Machado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor). Thus, in accordance with a further aspect of the invention, there is provided a method of treating a polyglutamine-repeat disease or CAG repeat expansion disease comprising administering to a patient, such as a mammal, especially a human, a therapeutically effective amount of a compound of the invention. In accordance with a further embodiment, there is provided a method of treating Huntington's disease (HD), dentatorubral-pallidoluysian atrophy (DRPLA), spinocerebellar ataxia type-1, spinocerebellar ataxia type-2, spinocerebellar ataxia type-3 (Machado-Joseph disease), spinocerebellar ataxia type-6, spinocerebellar ataxia type-7, or spinal and bulbar muscular atrophy, comprising administering to a patient, such as a mammal, especially a human, a therapeutically effective amount of a compound of the invention.

The basal ganglia are important for regulating the function of motor neurons; disorders of the basal ganglia result in movement disorders. Most prominent among the movement disorders related to basal ganglia function is Parkinson's disease (Obeso J A et al., Neurology., 2004 Jan 13;62(1 Suppl 1):S17-30). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. In one embodiment, the compounds of the invention may be used to treat movement disorders related to dysfunction of basal ganglia neurons.

PDE 10 inhibitors can be used to raise cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. In one embodiment, the compounds of the invention may be used to stop disease progression or restore damaged circuits in the brain by a combination of effects including increased synaptic plasticity, neurogenesis, anti-inflammatory, nerve cell regeneration and decreased apoptosis The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity will inhibit cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention may be used to stop the growth of cancer cells that express PDE10.

The compounds of the invention are also suitable for use in the treatment of diabetes and related disorders such as obesity, by focusing on regulation of the cAMP signaling system. By inhibiting PDE-10A activity, intracellular levels of cAMP and increased, thereby increasing the release of insulin-containing secretory granules and, therefore, increasing insulin secretion. See, for example, WO 2005/012485, which is hereby incorporated by reference in its entirety.

Thus, in accordance with a further aspect of the invention, there is provided a method of treating diabetes and related disorders comprising administering to a patient, such as a mammal, especially a human, a therapeutically effective amount of a compound of the invention. In accordance with a fuirther embodiment, there is provided a method of treating type 1 diabetes, type 2 diabetes, Syndrome X, impaired glucose tolerance, impaired fasting glucose, gestational diabetes, maturity-onset diabetes of the young (MODY), latent autoirimune diabetes adult (LADA), associated diabetic dyslipidemia, hyperglycemia, hyperinsulinemia, dyslipidemia, hypertriglyceridemia, obesity and insulin resistance, comprising administering to a patient, such as a mammal, especially a human, a therapeutically effective amount of a compound of the invention.

The compounds of the present invention may also be administered in combination with other known therapies for the treatment of diabetes, including, but not limited to, PPAR ligands (e.g. agonists, antagonists, such as Rosiglitazone, Troglitazone and Pioglitazone), insulin secretagogues (for example, sulfonylurea drugs (such as Glyburide, Glimepiride, Chlorpropamide, Tolbutamide, and Glipizide) and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forrns and formulations of insulin), and anti-obesity drugs (such as P-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat)).

When used in combination with one or more additional pharmaceutical agent or agents, the compounds of the present invention may be administered prior to, concurrently with, or following administration of the additional pharmaceutical agent or agents.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The compounds of the invention are typically administered at dosage levels and in a mammal customary for PDE10 inhibitors such as those known compounds mentioned above. For example, the compounds can be administered, in single or multiple doses, by oral administration at a dosage level of generally 0.001-100 mg/kg/day, for example, 0.01-100 mg/kg/day, preferably 0.1-70 mg/kg/day, especially 0.5-10 mg/kg/day. Unit dosage forms can contain generally 0.01-1000 mg of active compound, for example, 0.1-50 mg of active compound. For intravenous administration, the compounds can be administered, in single or multiple dosages, at a dosage level of, for example, 0.001-50 mg/kg/day, preferably 0.001-10 mg/kg/day, especially 0.01-1 mg/kg/day. Unit dosage forms can contain, for example, 0.1-10 mg of active compound.

In carrying out the procedures of the present invention, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be fuirther described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference in their entirety.

### EXAMPLES

All spectra were recorded at 300 MHz on a Bruker Instruments NMR unless otherwise stated. Coupling constants (J) are in Hertz (Hz) and peaks are listed relative to TMS (6 0.00 ppm). Microwave reactions were performed using a Personal Chemistry Optimizer™ microwave reactor in 10 mL Personal Chemistry microwave reactor vials. All reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C18 5 μm column using (i) a gradient of 20/80 to 80/20 acetonitrile (0.1% formic acid)/water (0.1% formic acid) over 6 min (Method A), (ii) a gradient of 20/80 to 80/20 acetonitrile (0.1% formic acid)/water (0.1% formnic acid) over 8 min (Method B), (iii) a gradient of 40/60 to 80/20 acetonitrile (0.1% formic acid)/water (0.1% formic acid) over 6 min (Method C), or (iv) a gradient of 40/60 to 80/420 acetonitrile (0.1% formic acid)/water (0.1% formic acid) over 8 min (Method D). Preparative HPLC was performed on 30 mm×100 mm Xtera Prep RP₁₈ 5 μp columns using an 8 min gradient of 95/5 to 20/80 water (0.1% formic acid)/acetonitrile (0.1% formic acid).

### Example 1

### Intermediate 6,7-Dimethoxycinnolin-4-ol.

1-(2-Amino-4,5-dimethoxyphenyl)ethanone (15.60 g, 0.07991 mol) was dissolved in concentrated hydrogen chloride in water (555 mL) and water (78 mL). The mixture was cooled to −5° C. (ice/brine) and a solution of sodium nitrite (5.55 g, 0.0804 mol) in water (20 mL) was added over a period of 45 min. The mixture was stirred another 1 h at 0° C. and then warmed to 60-75° C. for 4 h. The mixture was then cooled to room temperature using an ice bath and the resulting precipitate was collected via filtration. The solid HCl salt was added to about 1.0 L of water and then basified to pH ˜12 with NaOH. The resulting brown solution was neutralized with HCl and the resulting precipitate was collected to provide 12.77 g (78%) of 6,7-dimethoxycinnolin-4-ol as a light tan solid, which was used without further purification. MS [M+H]=207. ¹H NMR (DMSO d6) δ (ppm) 7.62 (s, 1H), 7.30 (s, 1H), 6.93(s, 1H), 3.89 (s, 3H), 3.85 (s, 3H),

### Example 2

### Intermediate 4-Chloro-6,7-dimethoxycinnoline.

Phosphoryl chloride (8.35 mL, 0.0896 mol) was added to 6,7-dimethoxycinnolin-4-ol (4.20 g, 0.0204 mol) with stirring. The warm yellow solution became a brick solid after 5 minutes. Additional phosphorus pentachloride (5.95 g, 0.0286 mol) was then added and the mixture was warmed to 50° C. for 15 min. The reaction mixture was cooled to room temperature and crushed ice was added (with a strong exotherm) to bring the volume to around 250 mL. The mixture was then neutralized to ˜pH 7 using saturated NaOAc and the resulting precipitate was collected by filtration and recrystallized from absolute ethanol (300 mL, boiling), to provide 3.81 g (83%) of 4-chloro-6,7-dimethoxycinnoline as superfine tan needles. MS [M+H]=225, ¹H NMR (DMSO d6) δ (ppm) 9.29 (s, 1H), 7.80 (s, 1H), 7.29 (s, 1H), 4.04 (s, 6H).

### Example 3

### Intermediate 4-bromo-6,7-dimethoxycinnoline.

Phosphorus oxybromide (12.2 g, 0.0426 mol) was added to 6,7-dimethoxycinnolin-4-ol (2.00 g, 0.00970 mol) in chloroform (20 mL). Brief solvation was observed for 10 min after addition of the POBr₃ then a suspension formed. The mixture was stirred for 8 h at room temperature, and was then heated to reflux for 18 h. The mixture was poured onto crushed ice (resulting in gas evolution), warmed to room temperature (giving a volume of around 125 mL) and neutralized to ˜pH 7 with saturated NaOAc. The mixture was then extracted with dichloromethane (5×50 mL) and the combined organics were dried (MgSO₄), filtered, and concentrated. Recrystallization from absolute ethanol provided 1.30 g (50%) of 4-bromo-6,7-dimethoxycinnoline as light yellow superfine fibrous crystals. MS [M+]=269, [M+2]=271, ¹H NMR (DMSO d6) δ (ppm) 9.38 (s, 1H), 7.77 (s, 1H), 7.21 (s, 1H), 4.03 (s, 6H).

### Example 4

### Intermediate (4,5-Dihydro-1-isopropyl-1H-inmidazol-2-yl)acetonitrile.

Ethyl 2-cyanoethanimidoate hydrochloride (500.00 mg, 3.3650 mmol) was dissolved in dry methylene chloride (5 mL) under an atmosphere of argon. N-isopropylethylenediamine (0.416 ml, 3.36 mmol) was added and the reaction was stirred for 18 hours. Saturated NaHCO₃ (20mL) was then added and the mixture was extracted with ethyl acetate (2×10 mL), washed with a saturated solution of NH₄Cl (2×10 mL), dried (MgSO₄), filtered, and concentrated to provide 313 mg (62%) of (4,5-dihydro-1-isopropyl-1H-imidazol-2-yl)acetonitrile as a light brown solid. MS [M+H]=152, ¹H NMR (CDCl₃) δ (ppm) 5.02(br s, 1H), 3.52 (m, 1H), 3.35 (m, 4H), 2.95 (s, 1H), 1.15 (s, 3H), 1.09 (s, 3H).

The following compound was prepared in a similar fashion with different starting materials:

[4,5-Dihydro-(4S)-4-phenyl-1,3-oxazol-2-yl]acetonitrile.

### Example 5

### Intermediate (1-Benzyl-4,5-dihydro-1H-imidazol-2-yl)acetonitrile.

Cyanoacetic acid, ethyl ester (1.42 mL, 0.0133 mol) and N-benzylethylenediamine (1.00 g, 0.00666 mol) were dissolved in 1,2-dimethylbenzene (50 mL). The reaction mixture was heated to reflux for 18 h with a dean-stark trap affixed. Upon cooling to room temperature, the entire mixture was loaded onto a 10 g SCX column, washed with MeOH (1 volume), eluted with 7M NH₃ (1 volume) in MeOH, and then concentrated to provide the crude product. Purification by rotary chromatography, using a gradient elution from 100% CHCl₃ to 10% MeOH in CHCl₃ provided 279 mg (21%) of (1-benzyl-4,5-dihydro-1H-imidazol-2-yl)acetonitrile as an orange solid. ¹H NMR (CDCl₃) δ (ppm) 7.30(m, 5H), 4.92 (br s, 1H), 4.21 (s, 2H), 3.53(m, 2H), 3.37 (m, 2H), 3.18 (s, 1H).

The following compound was prepared in a similar fashion with different starting materials:

(4,5-Dihydro-4,4-dimethyl-1-isopropyl-1H-imidazol-2-yl)acetonitrile MS [M+H]=180

### Example 6

### 109) (1-Benzyl4,5-dihydro-1H-inmidazol-2-yl)(6,7-dimethoxycinnolin-4-yl)acetonitrile.

(1-Benzyl-4,5-dihydro-1H-imidazol-2-yl)acetonitrile (106 mg, 0.534 mmol) and 4-chloro-6,7-dimethoxycinnoline (100 mg, 0.445 mmol) were placed in a dry flask under an atmosphere of argon. N,N-Dimethylformamide (3 mL) was added and a solution formed upon heating to 60° C. for 10 minutes. The mixture was then cooled to 0° C. and 2.67 mL of 0.500 M potassium bis(trimethylsilyl)amide in toluene was added dropwise over 5 min. The mixture was stirred for 14 h at room temperature. The entire mixture was then loaded onto a 10g SCX column and washed with MeOH (1 volume). Elution with 7M NH₃ (1 volume) in MeOH, followed by concentration on the rotovap provided the crude product, which was purified by prep HPLC/MS to provide 47 mg (28%) of (1-benzyl-4,5-dihydro-1H-imidazol-2-yl)(6,7-dimethoxycinnolin-4-yl)acetonitrile as an orange solid. MS [M+H]=388, ¹H NMR (CDCl₃) δ (ppm) 8.94 (s, 1H), 7.42 (s, 1H), 7.32 (m, 6H), 4.73 (s, 2H), 4.04 (s, 3H), 4.03(s, 3H), 3.65 (m, 4H).

The following compounds were prepared in a similar fashion with different starting materials:
111) (4,5-Dihydro-4,4-dimethyl-1-isopropyl-1H-imidazol-2-yl) (6,7-dimethoxycinnolin-4-yl)-acetonitrile, MS [M+H]=368
99) (6,7-Dimethoxycinnolin-4-yl)-((S)-4-phenyl-4,5-dihydro-oxazol-2-yl)-acetonitrile, MS [M+H]=375
84) (1-Benzyl-1H-imidazol-2-yl)(6,7-dimethoxycinnolin-4-yl)acetonitrile, MS [M+H]=386
85) (6,7-Dimethoxycinnolin-4-yl)(pyridin-3-yl)acetonitrile, MS [M+H]=307
86) (6,7-Dimethoxycinnolin-4-yl) [2-(trifluoromethyl)phenyl]acetonitrile, MS [M+H]=374
87) (4,5-Dihydro-1-isopropyl-1H-imidazol-2-yl) (6,7-dimethoxycinnolin-4-yl)acetonitrile, MS [M+H]=340

### Example 7

### 110) (4,5-Dihydro-4,4-dimethyl-i -isopropyl-1H-imidazol-2-yl) (6,7-dimethoxycinnolin-4-yl)-acetonitrile hydrochloride.

Crude (4,5-Dihydro-4,4-dimethyl-1-isopropyl-1H-imidazol-2-yl) (6,7-dimethoxycinnolin-4-yl)-acetonitrile (4.00 g, 0.0109 mol) was dissolved in ethyl acetate (250 mL) and the brown residue that remained was separated by decanting off the solution. 7.1 mL of 2.0 M hydrogen chloride in ether was added slowly to the solution with stirring, and the resultant yellow precipitate was collected by filtration to provide 4.15 g (94%) of (4,5-dihydro-4,4-dimethyl-1-isopropyl-1 H-imidazol-2-yl) (6,7-dimethoxycinnolin-4-yl)-acetonitrile hydrochloride as a yellow powder. mp 210-212° C. (dec). MS [M+H]=368, ¹H NMR (CDCl₃) δ (ppm) 14.79 (s, 1H), 11.59 (s, 1H), 8.20(s, 1H), 8.03 (s, 1H), 7.48 (s, 1H), 4.47 (m, 1H), 3.97 (s, 6H), 3.57 (s, 2H), 1.67 (s, 6H), 1.33 (s, 3H), 1.27 (s, 3H).

The following compounds were prepared in a similar fashion with different starting materials:
9) 4-(6,7-Dimethoxy-3,4-dihydroisoquinolin-2(1 H)-yl)-6,7-dimethoxycinnoline hydrochloride, MS [M+H]=382, LC/MS (EI) t_{R} 2.46 min (Method D)
58) 6,7-Dimethoxy-4-[7-(2-methoxyethoxy)-3,4-dihydroisoquinolin-2(1H)-yl]cinnoline hydrochloride, MS [M+H]=396, LC/MS (EI) t_{R} 2.18 min (Method C)
81) 2-(6,7-dimethoxycinnolin-4-yl)-1,2,3,4-tetrahydroisoquinoline-5-carboxylic acid hydrochloride MS [M+H]=366.2, LC/MS (EI) t_{R} 3.49 min (Method B)

### Example 8

### 83) (4,5-Dihydro4,4-dimethyl-1-isopropyl-1H-imidazol-2-yl) (6,7-dihydroxycinnolin-4-yl)-acetonitrile.

(4,5-Dihydro-4,4-dimethyl-1-isopropyl-1H-imidazol-2-yl) (6,7-dimethoxycinnolin-4-yl)-acetonitrile (20.0 mg, 0. 0544 mmol), N,N-dimethylformamide (1.00 mL, 0.0129 mol) and sodium ethanethiolate (45.8 mg, 0.544 mmol) were combined in a 10 mL sealed tube. The reaction was irradiated in a microwave on 300 watts, 200° C. for 600 seconds. The entire mixture was then loaded onto a 10 g SCX column and washed with MeOH (1 volume). Elution with 7M NH₃ (1 volume) in MeOH provided crude (4,5-dihydro-4,4-dimethyl-1-isopropyl-1H-imidazol-2-yl) (6,7-dihydroxycinnolin-4-yl)-acetonitrile as a reddish brown powder after concentration under reduced pressure. MS [M+H]=340, ¹H NMR (MeOD) δ (ppm) 8.60 (br. s, 1H), 7.97 (s, 1H), 6.88 (s,1H), 4.35 (m, 1H), 2.99 (s, 1H), 2.86 (s, 1H), 1.33 (s, 6H), 1.25 (s, 3H), 1,23 (s, 3H).

### Example 9

### 98) 4-(3,4-Dihydroisoquinolin-2(1H)-yl)-6,7-dimethoxycinnoline.

Into a dry 10 mL flask under argon was added 4-bromo-6,7-dimethoxycinnoline (50.0 mg, 0.186 mmol), 1,2,3,4-tetrahydroisoquinoline (27.9 uL, 0.223, mol), toluene (1.50 mL), tris(dibenzylideneacetone)dipalladium(0) (8 mg, 0.009 nmmol), 9,9-dimethyl-4,5-bis(diphenylphosphino)xanthene (11 mg, 0.018 mmol), and sodium tert-butoxide (26.8 mg, 0.279 mmol). The reaction mixture was heated to 50° C. for 8 h with stirring, and then cooled to room temperature and stirred for a fuirther 10 h. The entire reaction mixture was loaded onto a 10 g SCX column, washed with MeOH (1 volume), eluted with NH₃ in MeOH (7M), and concentrated to provide the crude product. Purification by rotary chromatography, using gradient elution from 100% chloroform to 10% methanol in chloroform, provided 36 mg (60%) 4-(3,4-dihydroisoquinolin-2(1H)-yl)-6,7-dimethoxycinnoline as a reddish oil, which was contaminated with ˜7% of the undesired reduction product (6,7-dimethoxycinnoline), as determined by ¹H NMR. MS [M+H]=322. ¹H NMR (CDCl₃) δ (ppm) 8.83 (s, 1H), 7.66(s, 1H), 7.22 (m, 4H), 7.12 (s, 1H), 4.50(s, 2H), 4.06 (s, 3H), 4.00 (s, 3H), 3.65 (t, J=6.0 Hz, 2H), 3.11 (t, J=6.0 Hz, 2H).

The following compounds were prepared in a similar fashion with different starting materials:
1) 6,7-Dimethoxy-4-[7-(trifluoromethyl)-3,4-dihydroisoquinolin-2(1H)-yl]cinnoline, MS [M+H]=390, LC/MS (EI) t_{R} 2.57 min (Method D)
2) 6,7-Dimethoxy-4-(5-methyl-2,3-dihydro-1H-indol-1-yl)cinnoline, MS [M+H]=322, LC/MS (EI) t_{R} 2.47 min (Method D)
3) 6,7-Dimethoxy-4-[7-(trifluoromethyl)-3,4-dihydroquinolin-1(2H)-yl]cinnoline, MS [M+H]=390, LC/MS (EI) t_{R} 3.51 min (Method D)
4) 6,7-Dimethoxy-4-(6-methyl-3,4-dihydroisoquinolin-2(1H)-yl)cinnoline, MS [M+H]=336, LC/MS (EI) t_{R} 2.55 min (Method D)
5) 6,7-Dimethoxy-4-(8-methyl-3,4-dihydroisoquinolin-2(1H)-yl)cinnoline, MS [M+H]=336, LC/MS (EI) t_{R} 2.54 min (Method D)
6) 4-(6,8-Dimethoxy-3,4-dihydroisoquinolin-2(1H)-yl)-6,7-dimethoxycinnoline, MS [M+H]=382, LC/MS (EI) t_{R} 2.55 min (Method D)
7) 6,7-Dimethoxy-4-(6-methyl-3,4-dihydroquinolin-1 (2H)-yl)cinnoline, MS [M+H]=336, LC/MS (EI) t_{R} 2.57 min (Method D)
8) 4-(6,7-Dimethoxy-3-methyl-3,4-dihydroisoquinolin-2(1H)-yl)-6,7-dimethoxycinnoline, MS [M+H]=396, LC/MS (EI) t_{R} 2.19 min (Method D)
10) 4-(5,7-Dimethoxy-3,4-dihydroisoquinolin-2(1H)-yl)-6,7-dimethoxycinnoline, MS [M+H]=382, LC/MS (EI) t_{R} 2.16 min (Method D)
11) 6,7-Dimethoxy-4-(5-methyl-3,4-dihydroisoquinolin-2(1H)-yl)cinnoline, MS [M+H]=336, LC/MS (EI) t_{R} 2.15 min (Method D)
12) 6,7-Dimethoxy-4-(7-methoxy-3,4-dihydroisoquinolin-2(1H)-yl)cinnoline, MS [M+H]=352, LC/MS (EI) t_{R} 2.27 min (Method D)
13) 6,7-Dimethoxy-4-(7-methyl-3,4-dihydroisoquinolin-2(1H)-yl)cinnoline, MS [M+H]=336, LC/MS (EI) t_{R} 2.27 min (Method D)
14) 6,7-Dimethoxy-4-(5-methoxy-3,4-dihydroisoquinolin-2(1H)-yl)cinnoline, MS [M+H]=352, LC/MS (EI) t_{R} 2.28 min (Method D)
15) Methyl 2-(6,7-dimethoxycinnolin-4-yl)-1,2,3,4-tetrahydroisoquinoline-6-carboxylate, MS [M+H]=380, LC/MS (EI) t_{R} 2.27 min (Method D)
16) 4-(6,7-Dihydrothieno[3,2-c]pyridin-5(4H)-yl)-6,7-dimethoxycinnoline, MS [M+H]=328, LC/MS (EI) t_{R} 2.26 min (Method D)
17) 6,7-Dimethoxy-4-[7-(2-methoxyethoxy)-3,4-dihydroisoquinolin-2(1H)-yl]cinnoline, MS [M+H]=396, LC/MS (EI) t_{R} 2.26 min (Method D)
19) 6,7-Dimethoxy-4-(6-nitro-2,3-dihydro-1H-indol-1-yl)cinnoline, MS [M+H]=353, LC/MS (EI) t_{R} 2.27 min (Method D)
20) Tert-butyl [1-(6,7-dimethoxycinnolin-4-yl)-1,2,3,4-tetrahydroquinolin-6-yl]carbamate, MS [M+H]=437, LC/MS (EI) t_{R} 2.76 min (Method D)
21) 6,7-Dimethoxy-4-(5-nitro-2,3-dihydro-1H-indol-1-yl)cinnoline, MS [M+H]=353, LC/MS (EI) t_{R} 2.94 min (Method D)
22) 4-(5-Fluoro-2,3-dihydro-1H-indol-1-yl)-6,7-dimethoxycinnoline, MS [M+H]=326, LC/MS (EI) t_{R} 2.11 min (Method D)
23) 1-(6,7-Dimethoxycinnolin-4-yl)-N,N-dimethylindoline-5-sulfonamnide, MS [M+H]=415, LC/MS (EI) t_{R} 2.20 min (Method D)
24) 6,7-Dimethoxy-4-(6-pyridin-4-yl-3,4-dihydroisoquinolin-2(1H)-yl)cinnoline, MS [M+H]=399, LC/MS (EI) t_{R} 1.96 min (Method D)
25) 6,7-Dimethoxy-4-(7-phenoxy-3,4-dihydroisoquinolin-2(1H)-yl)cinnoline, MS [M+H]=414, LC/MS (EI) t_{R} 2.61 min (Method D)
26) 4-(6,7-Dimethoxy-3,4-dihydroquinolin-1(2H)-yl)-6,7-dimethoxycinnoline, MS [M+H]=382, LC/MS (EI) t_{R} 2.28 min (Method D)
34) 4-(5,6-Dimethoxy-2,3-dihydro-1H-indol-1-yl)-6,7-dimethoxycinnoline, MS [M+H]=368, LC/MS (EI) t_{R} 2.22 min (Method D)
35) 6,7-Dimethoxy-4-[6-(1,3-thiazol-2-yl)-3,4-dihydroisoquinolin-2(1H)-yl]cinnoline, MS [M+H]=405, LC/MS (EI) t_{R} 2.30 min (Method D)
36) 1-(6,7-Dimethoxycinnolin-4-yl)-N,N-diethylindoline-5-sulfonamide, MS [M+H]=443, LC/MS (EI) t_{R} 2.72 min (Method D)
37) N-(Cyclopropylmethyl)-1-(6,7-dimethoxycinnolin-4-yl)indoline-5-sulfonamide, MS [M+H]=441, LC/MS (EI) t_{R} 2.55 min (Method C)
38) 1-(6,7-Dimethoxycinnolin-4-yl)-N-methylindoline-5-sulfonamide, MS [M+H]=401, LC/MS (EI) t_{R} 2.33 min (Method C)
39) 1-(6,7-Dimethoxycinnolin-4-yl)-N,N-dimethyl-1,2,3,4-tetrahydroquinoline-5-sulfonaniide, MS [M+H]=429, LC/MS (EI) t_{R} 2.54 min (Method C)
41) 4-(2,3-Dihydro-1H-indol-1-yl)-6,7-dimethoxycinnoline, MS [M+H]=308, LC/MS (EI) t_{R} 3.8 min (Method A)
42) 6,7-Dimethoxy-4-[5-(methylsulfonyl)-2,3-dihydro-1H-indol-1-yl]cinnoline, MS [M+H]=386, LC/MS (EI) t_{R} 3.7 min (Method A)
43) 4-[5-(3-Furyl)-2,3-dihydro-1H-indol-1-yl]-6,7-dimethoxycinnoline, MS [M+H]=374, LC/MS (EI) t_{R} 4.2 min (Method A)
44) 4-(1H-Indol-1-yl)-6,7-dimethoxycinnoline, MS [M+H]=306, LC/MS (EI) t_{R} 3.8 min (Method C)
47) 6,7-Dimethoxy-4-[5-(3-thienyl)-2,3-dihydro-1H-indol-1-yl]cinnoline, MS [M+H]=390, LC/MS (EI) t_{R} 2.8 min (Method C)
48) 6,7-Dimethoxy-4-(5-pyrimidin-5-yl-2,3-dihydro-1H-indol-1-yl)cinnoline, MS [M+H]=386, LC/MS (EI) t_{R} 3.6 min (Method A)
50) 1-(6,7-Dimethoxy-1-naphthyl)-N-ethylindoline-5-sulfonamide hydroformate, (isolated as the hydroformate salt from the free base by preparative HPLC using acetonitrile:water with 0.1% hydroformic acid) MS [M+H]=415, LC/MS (EI) t_{R} 4.01 min (Method B)
51) 1-(6,7-Dimethoxy-1-naphthyl)-N-isopropylindoline-5-sulfonamide hydroformate, (isolated as the hydroformate salt from the free base by preparative HPLC using acetonitrile:water with 0.1% hydroformic acid), MS [M+H]=429, LC/MS (EI) t_{R} 4.24 min (Method B)
52) N-cyclopropyl-1-(6,7-dimethoxycinnolin-4-yl)indoline-5-sulfonamide, MS [M+H]=427, LC/MS (EI) t_{R} 4.0 min (Method A)
53) 6,7-dimethoxy-4-[5-(pyrrolidin-1-ylsulfonyl)-2,3-dihydro-1H-indol-1-yl]cinnoline, MS [M+H]=441, LC/MS (EI) t_{R} 4.22 min (Method B)
54) 1-(6,7-dimethoxycinnolin-4-yl)-N,N-diisopropylindoline-5-sulfonamide MS [M+H]=471, LC/MS (EI) t_{R} 4.7 min (Method A)
55) 1-(6,7-dimethoxycinnolin-4-yl)-N-(2-methoxyethyl)indoline-5-sulfonamide MS [M+H]=445, LC/MS (EI) t_{R} 3.8 min (Method A)
56) 1-(6,7-dimethoxycinnolin-4-yl)-N-(2-morpholin-4-ylethyl)indoline-5-sulfonamide MS [M+H]=251, LC/MS (EI) t_{R} 2.5 min (Method A)
57) 6,7-dimethoxy-4-(5-pyridin-4-yl-2,3-dihydro-1H-indol-1-yl)cinnoline MS [M+H]=385, LC/MS (EI) t_{R} 2.5 min (Method A)
59) 4-[5-(3,5-dimethylisoxazol-4-yl)-2,3-dihydro-1H-indol-1-yl]-6,7-dimethoxycinnoline, MS [M+H]=403, LC/MS (EI) t_{R} 4.2 min (Method B).
88) 2-(6,7-Dimethoxycinnolin-4-yl)-1,2,3,4-tetrahydroisoquinoline-7-carbonitrile MS [M+H]=347
89) 4-(3,4-Dihydro-6-methoxy-isoquinolin-2(1H)-yl)-6,7-dimethoxycinnoline MS [M+H]=352
90) 4-(3,4-Dihydro-7-fluoro-isoquinolin-2(1H)-yl)-6,7-dimethoxycinnoline MS [M+H]=340
91) Methyl 2-(6,7-Dimethoxycinnolin-4-yl)-1,2,3,4-tetrahydroisoquinoline-5-carboxylate MS [M+H]=380
92) 4-(3,4-Dihydro-7-nitroisoquinolin-2(1H)-yl)-6,7-dimethoxycinnoline MS [M+H]=367
93) 4-(6,7-Diethoxy-3,4-dihydro-isoquinolin-2(1H)-yl)-6,7-dimethoxycinnoline MS [M+H]=410
94) 4-(3,4-Dihydro-5-nitroisoquinolin-2(1H)-yl)-6,7-dimethoxycinnoline MS [M+H]=367
95) 4-(1,3-Dihydro-2H-isoindol-2-yl)-6,7-dimethoxycinnoline MS [M+H]=308
96) Methyl 2-(6,7-dimethoxycinnolin-4-yl)-1,2,3,4-tetrahydroisoquinoline-7-carboxylate, MS [M+H]=380
97) 4-[4-(3-Chlorophenyl)piperazin-1-yl]-6,7-dimethoxycinnoline MS [M+H]=385
100) 4-(3,4-Dihydro-6,7-dimethoxy-isoquinolin-2(1H)-yl)-6,7-dimethoxycinnoline MS [M+H]=382
101) 4-(3,4-Dihydroquinolin-1(2H)-yl)-6,7-dimethoxycinnoline MS [M+H]=322
102) 6,7-Dimethoxy-(4-morpholin-4-yl)cinnoline MS [M+H]=276
103) 4-[4-(1,2-Benzisothiazol-3-yl)piperazin-1-yl]-6,7-dimethoxycinnoline MS [M+H]=408
104) 6,7-Dimethoxy-4-[(4aR,8aS)-octahydroisoquinolin-2(1H)-yl]cinnoline MS [M+H]=328
105) 4-{4-[Bis(4-fluorophenyl)methyl]piperazin-1-yl}-6,7-dimethoxycinnoline MS [M+H]=477
106) 6,7-Dimethoxy-4-piperidin-1-ylcinnoline, MS [M+H]=274
107) 4-[4-(1,3-Benzodioxol-5-ylmethyl)piperazin-1-yl]-6,7-dimethoxycinnoline, ¹H NMR (CDCl₃, 300 MHz) δ (ppm) 8.83 (s, 1H), 7.65 (s, 1H), 7.10 (s, 1H), 6.87 (s, 1H), 6.76 (s, 2H), 5.96 (s, 2H), 4.10 (s, 3H), 4.02 (s, 3H), 3.55 (s, 2H), 3.28 (m, 4H), 2.73 (m, 4H)
108) 6-(6,7-Dimethoxycinnolin-4-yl)-5,6,7,8-tetrahydro-[1,3]-dioxolo[4,5-g]isoquinoline, MS [M+H]=366
61) 6,7-dimethoxy-4-[5-(piperidin-1-ylsulfonyl)-2,3-dihydro-1 H-indol-1-yl]cinnoline, MS [M+H]=497.2, LC/MS (EI) t_{R} 5.53 min (Method B)
67) 6,7-bis(difluoromethoxy)-4-[7-(2-methoxyethoxy)-3,4-dihydroisoquinolin-2(1H)-yl]cinnoline, MS [M+H]=468.2, LC/MS (EI) t_{R} 5.06 min (Method B)
71) 6,7-dimethoxy-4-[6-(2-methoxyethoxy)-3,4-dihydroisoquinolin-2(1H)-yl]cinnoline, MS [M+H]=396.2, LC/MS (EI) t_{R} 3.77 min (Method B)
77) 6,7-dimethoxy-4-[7-(tetrahydrofuran-3-yloxy)-3,4-dihydroisoquinolin-2(1H)-yl]cinnoline, MS [M+H]=408, LC/MS (EI) t_{R} 2.05 min (Method C)
78) 6,7-dimethoxy-4-[7-(2-morpholin-4-ylethoxy)-3,4-dihydroisoquinolin-2(1H)-yl]cinnoline, MS [M+H]=451, LC/MS (EI) t_{R} 1.21 min (Method C)
79) 2-{[2-(6,7-dimethoxycinnolin-4-yl)-1,2,3,4-tetrahydroisoquinolin-5-yl]oxy} ethanol, MS [M+H]=382.2, LC/MS (EI) t_{R} 2.02 min (Method C)
80) 4-[7-[2-(benzyloxy)ethoxy]-3,4-dihydroisoquinolin-2(1H)-yl]-6,7-dimethoxycinnoline. MS [M+H]=472.3, LC/MS (EI) t_{R} 4.62 min (Method B)
82) 6,7-dimethoxy-4-[8-(2-methoxyethoxy)-3,4-dihydroisoquinolin-2(1H)-yl]cinnoline, MS [M+H]=396.2, LC/MS (EI) t_{R} 4.00 min (Method B)

### Example 10

### 18) 2-(6,7-Dimethoxycinnolin-4-yl)-7-methoxy-3,4-dihydroisoquinolin-1(2H)-one.

4-Bromo-6,7-dimethoxycinnoline (100.0 mg, 0.3716 mmol) and toluene (0.50 mL) were added to a dry 10 mL sealed tube under an atmosphere of argon. 7-Methoxy-3,4-dihydro-2H-isoquinolin-1-one (79.0 mg, 0.446 mmol), copper(I) iodide (4 mg, 0.02 mmol), potassium carbonate (103 mg, 0.743 mmol) and N,N′-dimethyl-1,2-ethanediamine (5.9 uL, 0.056 mmol) were added, and the reaction maintained for 24 hours at 115° C. The reaction mixture was cooled and filtered through a bed of celite. The celite was washed with chloroform, and the combined solutions were concentrated. Purification by rotary chromatography using a gradient elution from 100% chloroform to 10% methanol in chloroform provided 41 mg (30%) of 2-(6,7-dimethoxycinnolin-4-yl)-7-methoxy-3,4-dihydroisoquinolin-1(2H)-one as a yellow solid. MS [M+H]=366, LC/MS (EI) t_{R} 3.22 min (Method D). ¹H NMR (CDCl₃, 300 MHz) 6 (ppm) 9.15 (s, 1H), 7.84 (s, 1H), 7.74 (d, J=3 Hz, 1H), 7.33 (d. J=6 Hz, 1H), 7.17 (dd, J=3, 6 Hz, 1H), 6.97 (1H), 4.25 (m, 1H), 4.17 (s, 3H), 4.04 (s, 3H), 3.98 (m, 1H), 3.92 (s, 3H), 3.38 (m, 1H), 3.21 (m, 1H).

The following compounds were prepared in a similar fashion with different starting materials:
27) 2-(6,7-Dimethoxycinnolin-4-yl)-8-fluoro-7-methoxy-3,4-dihydroisoquinolin-1(2H)-one, MS [M+H]=384, LC/MS (EI) t_{R} 3.35 min (Method D)
28) 2-(6,7-Dimethoxycinnolin-4-yl)-6-fluoro-3,4-dihydroisoquinolin-1(2H)-one, MS [M+H]=354, LC/MS (EI) t_{R} 3.05 min (Method D)
29) 2-(6,7-Dimethoxycinnolin-4-yl)-7-fluoro-6-methoxy-3,4-dihydroisoquinolin-1(2H)-one, MS [M+H]=384, LC/MS (EI) t_{R} 3.11 min (Method D)
30) 7-(Cyclopropylmethoxy)-2-(6,7-dimethoxycinnolin-4-yl)-6-methoxy-3,4-dihydroisoquinolin-1(2H)-one, MS [M+H]=436, LC/MS (EI) t_{R} 3.27 min (Method D)
31) 2-(6,7-Dimethoxycinnolin-4-yl)-8-methoxy-2,3,4,5-tetrahydro-1H-2-benzazepin-1-one, MS [M+H]=380, LC/MS (EI) t_{R} 3.42 min (Method D)
32) 2-(6,7-Dimethoxycinnolin-4-yl)-7,8-dimethoxy-3,4-dihydroisoquinolin-1(2H)-one, MS [M+H]=396, LC/MS (EI) t_{R} 2.92 min (Method D)
33) 6-(Cyclopropylmethoxy)-2-(6,7-dimethoxycinnolin-4-yl)-7-methoxy-3,4-dihydroisoquinolin-1(2H)-one, MS [M+H]=436, LC/MS (EI) t_{R} 3.29 min (Method D)
40) 2-(6,7-Dimethoxycinnolin-4-yl)-6,7-dimethoxy-3,4-dihydroisoquinolin-1(2H)-one, MS [M+H]=396, LC/MS (EI) t_{R} 2.9 min (Method C)
69) 6-(benzyloxy)-2-(6,7-dimethoxycinnolin-4-yl)-3,4-dihydroisoquinolin-1(2H)-one, MS [M+H]=442, LC/MS (EI) t_{R} 4.01 min (Method C)
70) 2-(6,7-dimethoxycinnolin-4-yl)-5-hydroxy-3,4-dihydroisoquinolin-1(2H)-one, MS [M+H]=352, LC/MS (EI) t_{R} 2.26 min (Method C)
75) 2-(6,7-dimethoxycinnolin-4-yl)-5-(2-methoxyethoxy)-3,4-dihydroisoquinolin-1(2H)-one, MS [M+H]=410, LC/MS (EI) t_{R} 2.79 min (Method C)

### Example 11

### 45) 4-(1-Benzyl-1H-pyrazol4-yl)-6,7-dimethoxycinnoline

4-Bromo-6,7-dimethoxycinnoline (100 mg, 0.0004 mol), bis(triphenylphosphine)palladium(II) chloride (45.6 mg, 0.0650 mmol), 1-benzyl-1H-pyrazole-4-boronic acid (110 mg, 0.56 mmol), 133 μL of 2.00 M of sodium carbonate in water and 1 mL of dimethoxyethane:water:ethanol (7:3:2) were combined in a 10 mL sealed tube. The reaction was microwaved on 300 watts, 140° C. for 600 seconds. The reaction contents were filtered through a pad of Celite with MeOH, and concentrated. The residue was purified by ISCO chromatography with 50% EtOAc:Hex followed by 70:30:1 EtOAc:MeOH:NH₃ to give 25.4 mg (20%) of 4-(1-benzyl-1H-pyrazol-4-yl)-6,7-dimethoxycinnoline as a yellow solid. MS [M+H]=347, LC/MS (EI) t_{R} 5.11 min (Method B), ¹H NMR (CDCl₃, 300 MHz) δ (ppm) 9.05 (s, 1 H), 7.96 (s, 1 H), 7.76-7.75 (m, 2 H), 7.71-7.54 (m, 0.5 H), 7.52-7.46 (m, 0.5 H), 7.45-7.38 (m, 5 H), 5.45 (s, 2 H), 4.11 (s, 3 H), 3.96 (s, 3 H).

The following compounds were prepared in a similar fashion with different starting materials:
46) 6,7-dimethoxy-4-pyridin-3-ylcinnoline, MS [M+H]=268, LC/MS (EI) t_{R} 3.49 min (Method B)
60) 6,7-dimethoxy-4-(6-methoxy-2-naphthyl)cinnoline, MS [M+H]=347.2, LC/MS (EI) t_{R} 6.2 min (Method B)
64) 3-(6,7-dimethoxycinnolin-4-yl)-N-ethylbenzamide, MS [M+H]=338.1, LC/MS (EI) t_{R} 4.5 min (Method B)
65) 3-(6,7-dimethoxycinnolin-4-yl)-N-isobutylbenzamide, MS [M+H]=366.2, LC/MS (EI) t_{R} 5.41 min (Method B)
66) N-cyclopropyl-3-(6,7-dimethoxycinnolin-4-yl)benzamide, MS [M+H]=350.14, LC/MS (EI) t_{R} 4.62 min (Method B)
73) N-cyclohexyl-3-(6,7-dimethoxycinnolin-4-yl)benzamide, MS [M+H]=392.3, LC/MS (EI) t_{R} 5.93 min (Method B)
74) 3-(6,7-dimethoxycinnolin-4-yl)-N,N-diethylbenzamide, MS [M+H]=366.2, LC/MS (EI) t_{R} 5.09 min (Method B)
76) 4-(3,4-dihydronaphthalen-2-yl)-6,7-dimethoxycinnoline hydroformate, MS [M+H]=319.2, LC/MS (EI) t_{R} 6.17 min (Method B) (isolated as the hydroformate salt from the free base by preparative HPLC using acetonitrile:water with 0.1% hydroformic acid)

### Example 12

### 49) 6,7-Dimethoxy-4-(1,3-thiazol-2-yl)cinnoline

4-Bromo-6,7-dimethoxycinnoline (200 mg, 0.0007 mol), tetrakis(triphenylphosphine)palladium(0) (150 mg, 0.00013 mol) and 7 mL of 0.5 M bromo(1,3-thiazol-2-yl)zinc in tetrahydrofuran were added to a 10 mL sealed tube. The reaction was microwaved on 300 watts, 100° C. for 3600 seconds after which LC/MS showed the desired product. The entire mixture was concentrated and purified by ISCO chromatography with 50% EtOAc:Hex followed by 100% EtOAc as eluent to provide 3 mg (1%) of 6,7-dimethoxy-4-(1,3-thiazol-2-yl)cinnoline as a yellow solid. MS [M+H]=274, LC/MS (EI) t_{R} 5.29 min (Method B), ¹H NMR (CDCl₃, 300MHz) δ (ppm) 9.44 (s, 1 H), 8.55 (s, 8.22 (d, J=3.3 Hz, 1 H), 7.95 (d, J=3.3 Hz, 1 H),7.76 (s, 1 H), 4.12 (s, 3 H), 4.07 (s, 3 H).

### Example 13

### 62) 6,7-dimethoxy-N-(5-methyl4H-pyrazol-3-yl)cinnolin4-amnine hydroformate

4-Bromo-6,7-dimethoxycinnoline (530 mg, 2.0 mmol), 5-methyl-4H-pyrazol-3-amine (150 mg, 1.5 mmol), toluene (4 mL), tris(dibenzylideneacetone)dipalladium(0) (45 mg, 0.049 mmol), 9,9-dimethyl-4,5-bis(diphenylphosphino)xanthane (70.0 mg, 0.121 mmol) and sodium tert-butoxide (1.80E2 mg, 1.87 mmol) were combined in a 10 mL sealed tube. The reaction was irradiated in a microwave on 300 watts, at 140° C. for 600 seconds The reaction mixture was then filtered through Celite using methanol and methylene chloride and subsequently concentrated. The crude product was then dissolved in 1 mL methanol and filtered through a Gelman Acrodisk 0.45 micron HPLC filter. Purification using a C18 column preparative (30×00 mm) HPLC column and a gradient of 20-80% acetonitrile:water (with 0.1% formic acid) and a flow rate of 45 mL/min afforded 40.9 mg (8%) of 6,7-dimethoxy-N-(5-methyl-4H-pyrazol-3-yl)cinnolin-4-amine hydroformate as a yellow solid. MS [M+H]=286.1, LC/MS (EI) t_{R} 3.14 min (Method B), ¹H NMR (DMSO-d6) δ (ppm) d 12.57 (s, 1 H), 9.38 (s, 1 H), 7.94 (s, 1 H), 6.12 (s, 1.5 H), (s, 0.5 H), 4.01 (s, 3 H), 4.00 (s, 3 H), 2.30 (s, 3 H)

The following compound was prepared in a similar fashion with different starting materials:
63) 6,7-dimethoxy-N-(4-methyl-1,3-thiazol-2-yl)cinnolin-4-amine hydroformate, MS [M+H]=303.1, LC/MS (EI) t_{R} 3.82 min (Method B)

### Example 14

68) 2-(6,7-dimethoxycinnolin-4-yl)-6,7-dimethoxy-1,4-dihydroisoquinolin-3(2H)-one

N-(6,7-Dimethoxycinnolin-4-yl)-2-[2-(hydroxymethyl)-4,5-dimethoxyphenyl]acetamide (62.0 mg, 0.150 mmol), triethylamine (104 uL, 0.750 mmol) methanesulfonyl chloride (17 uL, 0.22 mmol) and methylene chloride (1 mL) were added to a dry flask under argon. The mixture was stirred at room temp for 16 hours, then poured into water. The product was extracted using ethyl acetate. The combined organic layers were washed with water, dried (MgSO₄), filtered, and concentrated to provide 2-(6,7-dimethoxycinnolin-4-yl)-6,7-dimethoxy-1,4-dihydroisoquinolin-3(2H)-one in 9.4%yield. MS [M+H]=346, LC/MS (EI) t_{R} 2.58 min (Method C), ¹H NMR (DMSO-d6) 6 (ppm) 9.11 (s, 1H), 7.80 (s, 1H), 6.81 (s, 1H), 6.73 (s, 1H), 6.70 (s, 1H), 5.00 (m. 2H), 4.67 (m, 2H), 4.11 (s, 3H), 3.95 (s, 3H), 3.88 (s, 6H)

### Example 15

### mPDElOA7 Enzyme Activity and Inhibition

To analyze the enzyme activity, 5 μL of serial diluted mmPDE10A7 containing lysate were incubated with equal volumes of diluted (100-fold) fluorescein labeled cAMP or cGMP for 30 minutes in MDC HE 96-well assay plates at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Device) was used to assess enzyme properties of mmPDE10A7. Data were analyzed with SoftMax Pro.

To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro. Compounds of the invention show activity with IC50 values of generally less than 5 μm.

### Example 16

### Apomorphine Induced Deficits in Prepulse Inhibition of the Startle Response in Rats, an in vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors, as they reduce the apormorphine-induced deficit in the prepulse inhibition of startle. Therefore, PDE10 inhibitors may be useful in restoring the deficits in sensorimotor gating that contribute to the thought disorders that characterize schizophrenia.

The preceding examples can be repeated with similar success by substituting the generically or specifically described reactants and/or operating conditions of this invention for those used in the preceding examples.

While the invention has been illustrated with respect to the production and of particular compounds, it is apparent that variations and modifications of the invention can be made without departing from the spirit or scope of the invention. Upon further study of the specification, further aspects, objects and advantages of this invention will become apparent to those skilled in the art.

1-97. (canceled)

98. A compound of Formula I or Formula II: 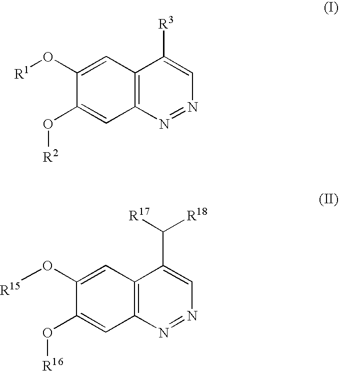
wherein:
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from formulas (a)-(h): 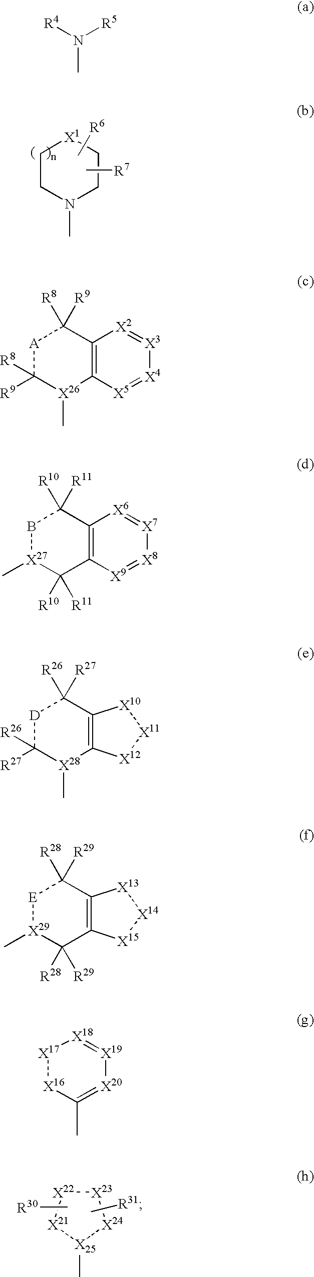
where:
n is 0, 1, 2, or 3;
-A-is a single bond, a double bond, —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁸—, ═CR⁸—CR⁸═, —CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, or ═CR⁸—CR⁸R⁹—CR⁸═;
—B— is a single bond, a double bond, —CR¹⁰R¹¹—, ═CR¹⁰—,—CR¹⁰═, —CR¹⁰R¹¹—CR¹⁰R¹¹—, ═CR¹⁰—CR¹⁰R¹¹—, —CR¹⁰R¹¹—CR¹⁰═, —CR¹⁰═CR¹⁰—, ═CR¹⁰—CR¹⁰═, —CR¹⁰R¹¹—CR¹⁰R¹¹—CR¹⁰R¹¹—, ═CR¹⁰—CR¹⁰R¹¹—CR¹⁰R¹¹—, —CR¹⁰═CR¹⁰—CR¹⁰R¹¹—, CR¹⁰R¹¹—CR¹⁰═CR¹⁰—, —CR¹⁰R¹¹—CR¹⁰R¹¹—CR¹⁰═, ═CR¹⁰—CR¹⁰═CR¹⁰—, —CR¹⁰═CR^{10—CR}¹⁰═, or ═CR¹⁰—CR¹⁰R¹¹—CR¹⁰═, OR ¹—CR -, with the proviso that when X²⁷ is N, then —B— is not a double bond, ═CR¹⁰—, ═CR¹⁰—CR¹⁰R¹¹—, ═CR¹⁰—CR¹⁰═, ═CR¹⁰—CR¹⁰R¹¹—CR¹⁰R¹¹—, ═CR¹⁰—CR¹⁰—, or ═CR¹⁰—CR¹⁰R¹¹—CR¹⁰═;
-D-is a single bond, a double bond, —CR²⁶R²⁷—, ═CR²⁶—, —CR²⁶═, —CR²⁶R²⁷—CR²⁶R²⁷—, ═CR²⁶—CR²⁶R²⁷—, —CR²⁶R²⁷ —CR²⁶═, —CR²⁶═CR²⁷—, ═CR²⁶—CR²⁶═, —CR²⁶R²⁷—CR²⁶R²⁷—CR²⁶R²⁷—, ═CR²⁶—CR²⁶R²⁷—CR²⁶R²⁷—, —CR²⁶═CR²⁶—CR²⁶R²⁷—, —CR²⁶R²⁷—CR²⁶═CR²⁶—, —CR²⁶R²⁷—CR²⁶R²⁷—CR²⁶═, ═CR²⁶—CR²⁶═CR²⁶—, —CR²⁶═CR²⁶—CR²⁶═, or ═CR²⁶—CR²⁶R²⁷—CR²⁶═;
-E-is a single bond, a double bond, —CR²⁸R²⁹—, ═CR²⁸—, —CR²⁸═, —CR²⁸R²⁹—CR²⁸R²⁹—, ═CR²⁸—CR²⁸R²⁹—, —CR²⁸R²⁹—CR²⁸═, —CR²⁸+CR²⁹—, ═CR²⁸—CR²⁸═, —CR²⁸R²⁹—CR²⁸R²⁹—CR²⁸R²⁹—, ═CR²⁸—C²⁸R²⁹—CR²⁸R²⁹—, —CR²⁸═C²⁸—CR²⁸R²⁹—, —CR²⁸R²⁹—CR²⁸═CR²⁸—,CR²⁸R²⁹—CR²⁸R²⁹—CR²⁸═, ═CR²⁸—CR²⁹═CR²⁸—, —CR²⁸═CR²⁸—CR²⁸═, or ═CR²⁸—CR²⁸R²⁹—CR²⁸═,
with the proviso that when X²⁹ is N, then -E-is not a double bond, ═CR²⁸—, ═CR²⁸—CR²⁸R²²⁹—, ═CR²⁸—CR²⁸═, CR²⁸—CR²⁸R²⁹—CR²⁸R²⁹—, ═CR²⁸—CR²⁸═CR²⁸—, or ═CR²⁸—CR²⁸R²⁹—CR²⁸═;
the dotted lines in formula (e) independently represent a single bond or a double bond, wherein there is at least one double bond between X¹⁰ and X¹¹ or X¹¹ and X¹²;
the dotted lines in formula (f) independently represent a single bond or a double bond, wherein there is at least one double bond between X¹³ and X¹⁴ or X¹⁴ and X¹⁵;
the dotted line in formula (g) independently represents a single bond or a double bond;
the dotted lines in formula (h) independently represent a single bond or a double bond, with the proviso that when two double bonds are present, they are not adjacent to each other;
R⁴ and R⁵ are each independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkysulphinyl, C₁₋₄- alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C═—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated CI-4 alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄alkylsulphonyl, —SO₂NHR¹², —NHSO₁₂R , —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹², or combinations thereof, or
R⁶ and R⁷ optionally form a cycloalkyl group, spiro or fused, having 3 to 8 carbon atoms, or
R ⁶ and R⁷ together with the carbon to which they are attached form a C(═O) group;
X¹ is O, S, NR¹³, CH₂, CHR⁶or CR⁶R⁷;
X², X³, X⁴, X⁵, X⁶, X⁷, X⁸, and X⁹ are each independently N or CR¹⁴, and wherein two adjacent X²-X⁹ groups can each be CR¹⁴ in which the two R¹⁴ groups are together a methylenedioxy, ethylenedioxy, difluoromethylenedioxy, or tetrafluoro-ethylenedioxy group, to form a fused ring structure;
x¹⁰, X¹¹, X¹², X¹³, X¹⁴, and X¹⁵ are each independently S, O, N, NR¹⁴, C(R¹⁴)₂, or CR¹⁴;
X¹⁶, X¹⁷, X¹⁸, X¹⁹, and X²⁰, are each independently N or CR¹⁴,
X¹⁶ and X¹⁷ can also each, independently, be NR¹⁴ or C(R¹⁴)₂, and
X¹⁸ and X¹⁹ or X¹⁹ and X²⁰ optionally form a fused aryl or heteroaryl, each of which may be substituted by one or more R¹⁴ groups;
X²¹, X²², X²³ , and X²⁴ are each independently O, S, N, NR¹⁴, CR¹⁴, or C(R¹⁴)₂;
X²⁵ is N, C or CR¹⁴;
wherein at least two of X²¹, X²², X²³, X²⁴, and X²⁵ are each, independently, O, S, N, or NR¹⁴;
X²⁶ is N or CR⁸;
X²⁷is C, N, or CR¹⁰;
X²⁸ is N or CR²⁶;
X²1 is C, N, or CR²;
R⁸, R⁹, R¹⁰, R¹¹, R²⁶ , R²⁷ , R²⁸, and R²⁹ are, in each case, independently:
absent, H, or alkyl having 1 to 8 carbon atoms, cycloalkyl having 3 to 12 carbon atoms, or cycloalkylalkyl having 4 to 12 carbon atoms, each of which is branched or unbranched and which is unsubstituted or substituted one or more times with halogen, C₁₋₄-alkyl, Cl-₄-alkoxy, oxo, or combinations thereof; or
R⁸ and R⁹, R¹⁰ and R¹¹, R²⁶ and R²⁷, and/or R²⁸ and R²⁹ together optionally-form a cycloalkyl group, spiro or fused, having 3 to 8 carbon atoms, or
one or more of R⁸and R⁹and the carbon atom to which they are attached, or one or more of RR¹⁰ and R¹¹ and the carbon atom to which they are attached, or one or more of R²⁶ and R²⁷ and the carbon atom to which they are attached, or one or more of R²⁸ and R²⁹ and the carbon atom to which they are attached, in each case form a C(═O) group;
R¹² is H or alkyl having 1 to 8 carbon atoms, cycloalkyl having 3 to 12 carbon atoms, or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or combinations thereof;
R¹³ is H,
straight, branched or cyclic alkyl having up to 12 carbon atoms which is unsubstituted or substituted one or more times by halogen, hydroxy, C₁₋₄-alkoxy, halogenated C₁₌₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —Ch₂— groups is, in each case independently, replaced by —O—, —S—, or —NH—, and wherein optionally one or more —CH₂CH₂— groups is replaced in each case by —CH═CH— or —C—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
—CH(aryl)₂ wherein each aryl group has 6 to 14 carbon atoms and is unsubstituted or substituted one or more times by halogen, C₁₋₄ alkyl, halogenated CA4 alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, Cl ₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxy-carbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄alkylsulphonyl, —SO₂NHR¹², —NHSO₂R¹², —NR¹²COR¹², —CONHR¹², —NHCONHR¹², —OCONHR , —NHCOOR¹², —SCONHR¹², —SCSNHR¹², or —NHCSNHR¹² or combinations thereof, wherein optionally one or more —Ch₂— groups is, in each case independently, replaced by —O—, —S—, or —NH—, and wherein optionally one or more —CH₂CH₂- groups is replaced in each case by —CH═CH— or —C—C—,
a heterocyclic group, which is saturated, partially saturated, or unsaturated, having 5 to 10 ring atoms in which at least 1 ring atom is a heteroatom which is unsubstituted or substituted one or more times by halogen, C₆₋₁₄ aryl, arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
aryloxy having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryloxy having 5 to 10 ring atoms in which at least 1 ring atom is a heteroatom which is unsubstituted or substituted one or more times by halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
O-heterocyclic group, in which the heterocyclic group is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof;
O-heterocyclicalkyl group, in which the heterocyclic group is nonaromatic, having 5 to 10 ring atoms in which at least 1 ring atom is a heteroatom, and the alkyl portion has 1 to 3 carbon atoms and the heterocyclic group is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof; or
halogen, hydroxy, C₁₋₄-alkoxy, C₁₋₄-alkyloxyC₁₋₄-alkoxy, C₄₋₁₂-cycloalkylalkyloxy, C₁₋₄-alkyloxyC₇₋₁₆-arylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —SO₂NHR²⁵, —SO₂NR¹⁹R²⁵, —SO₂R³², —NHSO₂R¹², —NR¹²COR¹², —CONHR¹²R²⁵, —CONHCH₂CH(CH₃)₂, CONH-cycloalkyl, —CONR¹²R²⁵, C₁₋₄alkyl-CONR¹²R²⁵, —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂- groups is replaced in each case by —CH═CH— or —C—C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
R¹⁹ is H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof;
R²⁵ is H,
alkyl having 1 to 8 carbon atoms, which is branched or unbranched and which is unsubstituted or substituted one or more times with halogen, C₁₋₄-alkyl, C₁₋₄-alkoxy, oxo, or combinations thereof;
cycloalkyl having 3 to 10 carbon atoms, which is unsubstituted or substituted one or more times with halogen, C₁₋₄-alkyl, C₁₋₄-alkoxy, oxo, or combinations thereof,
cycloalkylalkyl having 4-12 carbon atoms which is unsubstituted or substituted one or more times with halogen, C₁₋₄-alkyl, C₁₋₄-alkoxy, oxo, or combinations thereof,
heterocyclic group which is saturated, partially saturated, or unsaturated, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof; or
heterocyclicalkyl group wherein the heterocyclic group has 5 to 10 ring atoms in which at least 1 ring atom is a heteroatom and the alkyl portion has 1 to 4 carbon atoms, the heterocyclic group is unsubstituted or is substituted one or more times by halogen C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof;
R³⁰ and R³¹ are, in each case, independently:
H or alkyl having 1 to 8 carbon atoms, cycloalkyl having 3 to 12 carbon atoms or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or combinations thereof; or
R³⁰ and R³¹ form a cycloalkyl group, spiro or fused, having 3 to 8 carbon atoms, or R³⁰ and R³¹ and the carbon atom to which they are attached form a C(═O) group; and
R³² is a heterocyclic group which is saturated or partially saturated and has 5 to 10 ring atoms in which at least 1 ring atom is a heteroatom and which is unsubstituted or substituted one or more times by halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof; or
a solvate, a single enantiomer, a single diastereomer, a mixture of enantiomers, a mixture of diastereomers, a polymorph, or a pharmaceutically acceptable salt thereof,
with the proviso that said compound is not:
4-(4-methoxyanilino)-6,7-dimethoxycinnoline,
4-(4-ethoxyanilino)-6,7-dimethoxycinnoline,
4-(4-methylanilino)-6,7-dimethoxycinnoline,
4-(3,4-dimethylanilino)-6,7-dimethoxycinnoline,
4-(2-chloroanilino)-6,7-dimethoxycinnoline,
4-(3-chloroanilino)-6,7-dimethoxycinnoline,
4-(4-chloroanilino)-6,7-dimethoxycinnoline,
4-(3-bromoanilino)-6,7-dimethoxycinnoline,
4-(3-hydroxy-4-methylanilino)-6,7-dimethoxycinnoline,
4-(2-fluoro-5-hydroxy-4-methylanilino)-6,7-dimethoxycinnoline,
4-(4-chloro-2-fluoro-5-hydroxyanilino)-6,7-dimethoxycinnoline,
6,7-dimethoxy-4-(1-piperazinyl)cinnoline,
4-amino-6,7-dimethoxycinnoline,
4-anilino-6,7-dimethoxycinnoline,
6,7-dimethoxy-α-1-naphthyl-4-cinnoline-acetonitrile,
4-(4-aminobenzyl)-6,7-dimethoxy-cinnoline,
6,7-dimethoxy-α-(3-methoxyphenyl)-4-cinnoline-acetonitrile,
α-[4,5-dihydro-4,4-dimethyl-1-(1-methylethyl)-1 H-imidazol-2-yl]-6,7-dimethoxy-4-cinnolineacetonitrile,
α-(3,4-dimethoxyphenyl)-6,7-dimethoxy-4-cinnoline-acetamide,
6,7-dimethoxy-α-phenyl-4-cinnoline-acetonitrile,
α-(3,4-dimethoxyphenyl)-6,7-dimethoxy-4-cinnoline-acetonitrile,
6,7-dimethoxy-α-(4-iodophenyl)-4-cinnoline-acetonitrile,
6,7-dimethoxy-α-(4-bromophenyl)-4-cinnoline-acetonitrile,
6,7-dimethoxy-α-(4-chlorophenyl)-4-cinnoline-acetonitrile,
α-(3,4-dichlorophenyl)-6,7-dimethoxy-4-cinnoline-acetonitrile,
6,7-dimethoxy-α-(phenyl)-4-cinnoline-acetamide (also called α-(6,7-dimethoxy-4-cinnolyl)phenylacetamide),
α-(4-aminophenyl)-6,7-dimethoxy-4-cinnolinea-cetonitrile, or
4-benzyl-6,7-dimethoxycinnoline,
or a pharmaceutically acceptable salt thereof, or a solvate thereof, or a solvate of a pharmaceutically acceptable salt thereof.

99. The compound according to claim 98, wherein R³ is of formula (a).

100. The compound according to claim 98, wherein R³ is of formula (b).

101. The compound according to claim 98, wherein R³ is of formula (c).

102. The compound according to claim 98, wherein R³ is of formula (d).

103. The compound according to claim 98, wherein R³ is of formula (e).

104. The compound according to claim 98, wherein R³ is of formula (f).

105. The compound according to claim 98, wherein R³ is of formula (g).

106. The compound according to claim 98, wherein R³ is of formula (h).

107. The compound according to claim 98, wherein:
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from: 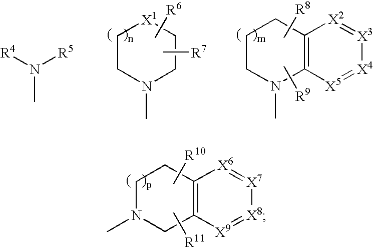
n is 0, 1, 2, or 3;
m is 0, 1, 2, or 3;
p is 0, 1, 2, or 3;
R⁴ and R⁵ are each independently:
H,
straight, branched or cyclic alkyl having up to 12 carbon atoms, which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, Cl 4-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C═C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR¹², or combinations thereof, or
R⁶ and R⁷ optionally form a cycloalkyl group, spiro or fused, having 3 to 8 carbon atoms,
X¹is O, S, NR¹³, CH₂, CHR⁶ or CR⁶R⁷;
X², X³, X⁴, X⁵, X⁶, X⁷, X⁸ , and X⁹ are each independently N or CR¹⁴, and wherein two adjacent X²-X⁹ groups can together be a methylenedioxy, ethylenedioxy group, difluoromethylenedioxy, or tetrafluoromethylenedioxy, to form a fused ring structure;
R⁸ and R⁹ are in each case independently:
H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof, or R⁸ and R⁹ form a cycloalkyl group, spiro or fused, having 3 to 8 carbon atoms;
R¹⁰ and R¹¹ are in each case independently:
H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof, or R¹⁰ and R¹¹ form a cycloalkyl group, spiro or fused, having 3 to 8 carbon atoms;
R¹² is H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof;
R¹³ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
—CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C24-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C═C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is:
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, Cl 4-alkoxy, halogenated C₁₋₄ alkoxy, nitro, oxo, amino, C14-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₂₋₄-alkylsulphinyl, C₁₋₄-alkylsulphonyl, phenoxy, or combinations thereof;
R¹⁸ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹², —CONHR¹², —NHCONHR¹⁹, —OCONHR¹², —NHCOOR¹², —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, CIX₄-alkylsulphonyl, —SO₂NHR¹⁹, —NHSO₂R¹⁹, —NR COR¹², —CONHR¹⁹, —NHCONHR¹⁹, —OCONHR¹⁹, —NHCOOR¹⁹, —SCONHR¹⁹, —SCSNHR¹², or —NHCSNHR¹⁹, or combinations thereof;
R¹⁹ is H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof; or or a pharmaceutically acceptable salt thereof.

108. The compound according to claim 98, wherein said compound is a compound of Formula I or a pharmaceutically acceptable salt thereof.

109. The compound according to claim 108, wherein when n is 1 and X¹ is NH, and R⁶ and R⁷ are not both H.

110. The compound according to claim 108, wherein when one of R⁴ or R⁵ is H, phenyl, or phenyl substituted by alkyl, hydroxyl and/or halogen, the other is not H.

111. The compound according to claim 108, wherein when n is 1 and X¹ is NH, R⁶ and R⁷ are not both H, and when one of R⁴ or R⁵ is H, phenyl, or phenyl substituted by alkyl, hydroxyl and/or halogen, the other is not H.

112. The compound according to claim 108, wherein when one of R⁴ and R⁵ is H or substituted or unsubstituted phenyl, the other is not H.

113. The compound according to claim 108, wherein —NR⁴R⁵ is not NH₂, unsubstituted monoalkylamino, or substituted or unsubstituted anilino.

114. The compound according to claim 108, wherein —NR⁴R⁵ is not NH₂, unsubstituted monoalkylamino, unsubstituted dialkylamino, or substituted or unsubstituted anilino.

115. The compound according to claim 98, wherein when R¹⁸ is cyano, then R¹⁷ is other than halo-substituted phenyl.

116. The compound according to claim 98, wherein R¹⁸ is other than H.

117. The compound according to claim 98, wherein R¹⁸ is not H, cyano, or —CONHR¹⁹.

118. The compound according to claim 98, wherein R³ is: 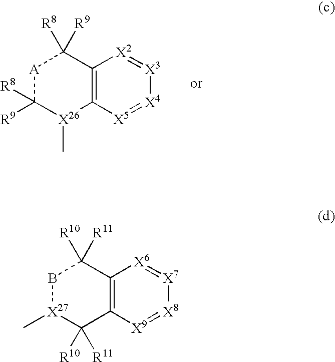
in which X²,X³, X⁴,X⁵,X⁶, X⁷, X⁸, and X⁹ are each CR¹⁴,and R¹⁴ is H, CH₃, CN, F, CF₃, OCH₂-cyclopropyl, OCH₃, OC₂H₅, CH₂OH, OCH₂CH₂OH, OCH₂CH₂OCH₃, SO₂NHCH₂-cyclopropyl, S0₂N(CH₃)₂, a heterocyclic group, or CO₂CH₃. SO₂NHCH₃, SO₂NHCH₂-cyclopropyl, SO₂N(CH₃)₂, a heterocyclic group, or CO₂CH₃.

119. The compound according to claim 98, wherein X²⁶ is N.

120. The compound according to claim 118, wherein X²⁶ is N.

121. The compound according to claim 98 wherein X²⁷ is N or CR¹⁰.

122. The compound according to claim 118 wherein X²⁷ is N or CR¹⁰.

123. A compound according to claim 122, wherein X²⁷ is N.

124. The compound according to claim 98, wherein R⁸, R⁹, R¹⁰, and R¹¹ are each H or CH₃.

125. The compound according to claim 124, wherein R⁸, R⁹, R¹⁰, and R¹¹ are each H.

126. A compound according to claim 98, wherein R³ is of formula (c) and one set of R⁸ and R⁹ together with the carbon to which they are attached form a C(═O) group.

127. A compound according to claim 98, wherein R³ is of formula (d) and one set of R¹⁰ and R¹¹ together with the carbon to which they are attached form a C(═O) group.

128. A compound according to claim 98, wherein R³ is of formula (c) or formula (d) and R¹⁴ is H, halogen, alkoxy, alkoxyalkyl, cycloalkylalkyloxy, or alkyloxyalkoxy.

129. A compound according to claim 98, wherein R³is of formula (c) and -A- represents a single bond, a double bond, —CR⁸R⁹—, ═CR⁸—, or —CR⁸═.

130. A compound according to claim 98, wherein R³is of formula (d) and —B— represents a single bond, —CR¹⁰R¹¹—, or —CR°═.

131. A compound according to claim 98, wherein R³ is of formula (e) or formula (f), -D- is a single bond, a double bond, —CR²⁶R²⁷—, ═CR²⁶—, or —CR²⁶═, and -E- is a single bond, —CR²⁸R²⁹—, or —CR²⁸50 .

132. A compound according to claim 98, wherein R²⁶, R²⁷, R²⁸, R²⁹, R³⁰, and R³¹ are each H or CH₃.

133. A compound according to claim 98, wherein R³⁰ and R³¹ together with the carbon to which they are attached form a C(═O) group.

134. A compound according to claim 98, wherein R³is of formula (h) and contains no double bonds.

135. A compound according to claim 98, wherein R³ is of formula (h) and contains two non-adjacent double bonds.

136. A compound according to claim 98, wherein R³ is of formula (f) and -E- is CR²⁸R²⁹—, R²⁸ and R²⁹ are H, X¹³ and X¹⁴ are N, and X¹⁵ is CR¹⁴ .

137. A compound according to claim 98, wherein R³ is of formula (g) and X¹⁶, X¹⁷, X¹⁸, X¹⁹, and X²⁰ are C or CR¹⁴, or one of X¹⁶, X¹⁷, X¹⁸, X¹⁹, and X²⁰ is N and the rest are C or CR¹⁴.

138. A compound according to claim 98, wherein R is of formula (g) and:X¹⁶, X¹⁷, X¹⁸ and X²⁰ are CR¹⁴ and X¹⁹ is N; X¹⁶, X¹⁷, X¹⁸ and X²⁰ are CH and X¹⁹ is CR¹⁴; or X¹⁶, X¹⁷ and X²⁰ are CR¹⁴ (e.g., CH) and X¹⁸ and X¹⁹ form a fused aryl.

139. A compound according to claim 98, wherein R³ is of formula (h) and: X²³ is N or NR¹⁴, X²⁵ is N, and X²¹, X²² and X²⁴ are C or CHR¹⁴; X²² and X²³ are N or NR¹⁴ and X²¹, X²⁴, and X²⁵ are C or CR¹⁴; or X²¹ is S, X²⁴ is N, X²² and X²³ are CR¹⁴ and X²⁵ is C.

140. A compound according to claim 98, wherein R¹⁵ and R¹⁶ are each alkyl having 1 to 4 carbon atoms, which is unsubstituted or substituted one or more times by halogen.

141. A compound according to claim 98, wherein R¹ and R² are each alkyl having 1 to 4 carbon atoms, which is unsubstituted or substituted one or more times by halogen.

142. The compound according to claim 98, wherein the compound is a compound of Formula I and R³ is (b).

143. The compound according to claim 98, wherein the compound is a compound of Formula I and R³ is (g).

144. The compound according to claim 98, wherein the compound is a compound of Formula I and R³ is (h).

145. A compound of Formula III: 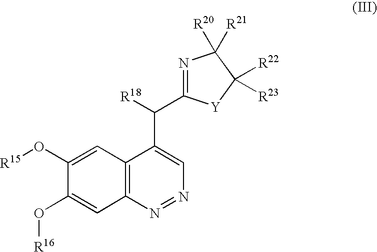
wherein:
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁸ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR ¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹², —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹, or combinations thereof;
Y is NR¹², O or S;
R²⁰, R²¹ R²², and R²³ are independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least I ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy; halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof, or
cyano, carboxy, C₁₋₄-hydroxyalkyl, COR¹⁹, COOR¹⁹, CONHR¹⁹ or combinations thereof,
wherein two of R²⁰, R²¹, R²² , and R²³ together may optionally form a spiro or fused cycloalkyl group having 3 to 8 carbon atoms, and R²⁰ and R²¹ or R²² and R²³ together may optionally form an oxo group;
R²⁴ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHS₂R¹⁹, —NR¹⁹COR¹⁹, —CONHR¹⁹, —NHCONHR¹⁹, —OCONHR¹⁹, —NHCOOR¹, —SCONHR¹⁹, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C1 ₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof; or
or a pharmaceutically acceptable salt or solvate thereof, or a solvate of a pharmaceutically acceptable salt thereof,
wherein said compound is not α-[4,5-dihydro-4,4-dimethyl-1-(1-methylethyl)-1H-imidazol-2-yl]-6,7-dimethoxy-4-cinnolineacetonitrile, or a pharmaceutically acceptable salt or solvate thereof, or a solvate of a pharmaceutically acceptable salt thereof.

146. The compound according to claim 145, wherein said compound is in the form of a single enantiomer, a single diastereomer, a mixture of enantiomers, a mixture of diastereomers, or a polymorph.

147. The compound according to claim 145, wherein if R²⁴ is isopropyl, then R²⁰ and R²¹ are not both methyl.

148. The compound according to claim 145, wherein if R²⁴ is isopropyl, then R²⁰ and R²¹ are not both alkyl.

149. The compound according to claim 145, wherein R¹⁸ is other than H.

150. The compound according to claim 145, wherein R18 is not H, cyano, or —CONHR¹⁹.

151. The compound according to claim 145, wherein Y is NR²⁴ or 0, and R²⁰ and R²¹ are each H, CH₃ or phenyl.

152. The compound according to claim 145, wherein R²² and R²³ are each H or CH₃.

153. The compound according to claim 145, wherein R²⁴ is cyclopropyl, benzyl or cyclopropylmethyl.

154. A compound according to claim 98, wherein said compound is:
6,7-Dimethoxy-4-[7-(trifluoromethyl)-3,4-dihydroisoquinolin-2(1H)-yl]cinnoline;
6,7-Dimethoxy-4-(5-methyl-2,3-dihydro-1H- indol-1-yl)cinnoline;
6,7-Dimethoxy-4-[7-(trifluoromethyl)-3,4-dihydroquinolin-1(2H)-yl]cinnoline;
6,7-Dimethoxy-4-(6-methyl-3,4-dihydroisoquinolin-2(1H)-yl)cinnoline;
6,7-Dimethoxy-4-(8-methyl-3,4-dihydroisoquinolin-2(1H)-yl)cinnoline;
4-(6,8-Dimethoxy-3,4-dihydroisoquinolin-2(1H)-yl)-6,7-dimethoxycinnoline;
6,7-Dimethoxy-4-(6-methyl-3,4-dihydroquinolin-1 (2H)-yl)cinnoline;
4-(6,7-Dimethoxy-3-methyl-3,4-dihydroisoquinolin-2(1 H)-yl)-6,7-dimethoxycinnoline;
4-(6,7-Dimethoxy-3,4-dihydroisoquinolin-2(1 H)-yl)-6,7-dimethoxycinnoline;
4-(5,7-Dimethoxy-3,4-dihydroisoquinolin-2(1 H)-yl)-6,7-dimethoxycinnoline;
6,7-Dimethoxy-4-(5-methyl-3,4-dihydroisoquinolin-2(1H)-yl)cinnoline;
6,7-Dimethoxy-4-(7-methoxy-3,4-dihydroisoquinolin-2(1H)-yl)cinnoline;
6,7-Dimethoxy-4-(7-methyl-3,4-dihydroisoquinolin-2(1H)-yl)cinnoline;
6,7-Dimethoxy-4-(5-methoxy-3,4-dihydroisoquinolin-2(1H)-yl)cinnoline;
Methyl 2-(6,7-dimethoxycinnolin-4-yl)-1,2,3,4-tetrahydroisoquinoline-6-carboxylate;
4-(6,7-Dihydrothieno[3,2-c]pyridin-5(4H)-yl)-6,7-dimethoxycinnoline;
6,7-Dimethoxy-4-[7-(2-methoxyethoxy)-3,4-dihydroisoquinolin-2(1H)-yl]cinnoline;
2-(6,7-Dimethoxycinnolin-4-yl)-7-methoxy-3,4-dihydroisoquinolin-1(2H)-one;
6,7-Dimethoxy-4-(6-nitro-2,3-dihydro-1H-indol-1-yl)cinnoline;
Tert-butyl [1-(6,7-dimethoxycinnolin-4-yl)-1,2,3,4-tetrahydroquinolin-6-yl]carbamate;
6,7-Dimethoxy-4-(5-nitro-2,3-dihydro-1H-indol-1-yl)cinnoline;
4-(5-Fluoro-2,3-dihydro-1H-indol-1-yl)-6,7-dimethoxycinnoline;
1-(6,7-Dimethoxycinnolin-4-yl)-N,N-dimethylindoline-5-sulfonamide;
6,7-Dimethoxy-4-(6-pyridin-4-yl-3,4-dihydroisoquinolin-2(1H)-yl)cinnoline;
6,7-Dimethoxy-4-(7-phenoxy-3,4-dihydroisoquinolin-2(1H)-yl)cinnoline;
4-(6,7-Dimethoxy-3,4-dihydroquinolin-1(2H)-yl)-6,7-dimethoxycinnoline;
2-(6,7-Dimethoxycinnolin-4-yl)-8-fluoro-7-methoxy-3,4-dihydroisoquinolin-1(2H)-one;
2-(6,7-Dimethoxycinnolin-4-yl)-6-fluoro-3,4-dihydroisoquinolin-1(2H)-one;
2-(6,7-Dimethoxycinnolin-4-yl)-7-fluoro-6-methoxy-3,4-dihydroisoquinolin-1(2H)-one;
7-(Cyclopropylmethoxy)-2-(6,7-dimethoxycinnolin-4-yl)-6-methoxy-3,4-dihydroisoquinolin-1(2H)-one;
2-(6,7-Dimethoxycinnolin-4-yl)-8-methoxy-2,3,4,5-tetrahydro-1H-2-benzazepin-1-one;
2-(6,7-Dimethoxycinnolin-4-yl)-7,8-dimethoxy-3,4-dihydroisoquinolin-1(2H)-one;
6-(Cyclopropylmethoxy)-2-(6,7-dimethoxycinnolin-4-yl)-7-methoxy-3,4-dihydroisoquinolin-1(2H)-one;
4-(5,6-Dimethoxy-2,3-dihydro-1H-indol-1-yl)-6,7-dimethoxycinnoline;
6,7-Dimethoxy-4-[6-(1,3-thiazol-2-yl)-3,4-dihydroisoquinolin-2(1H)-yl]cinnoline;
1-(6,7-Dimethoxycinnolin-4-yl)-N,N-diethylindoline-5-sulfonamide;
N-(Cyclopropylmethyl)-1-(6,7-dimethoxycinnolin-4-yl)indoline-5-sulfonamide;
1-(6,7-Dimethoxycinnolin-4-yl)-N-methylindoline-5-sulfonamide;
1-(6,7-Dimethoxycinnolin-4-yl)-N,N-dimethyl-1,2,3,4-tetrahydroquinoline-5-sulfonamide;
2-(6,7-Dimethoxycinnolin-4-yl)-6,7-dimethoxy-3,4-dihydroisoquinolin-1 (2H)-one 4-(2,3-Dihydro-1H-indol-1-yl)-6,7-dimethoxycinnoline;
6,7-Dimethoxy-4-[5-(methylsulfonyl)-2,3-dihydro-1H-indol-1-yl]cinnoline;
4-[5-(3-Furyl)-2,3-dihydro-1H-indol-1-yl]-6,7-dimethoxycinnoline;
4-(1 H-Indol-1-yl)-6,7-dimethoxycinnoline;
4-(1-Benzyl-1H- pyrazol-4-yl)-6,7-dimethoxycinnoline;
6,7-Dimethoxy-4-pyridin-3-ylcinnoline;
6,7-Dimethoxy-4-[5-(3-thienyl)-2,3-dihydro-1H-indol-1-yl]cinnoline;
6,7-Dimethoxy-4-(5-pyrimidin-5-yl-2,3-dihydro-1H-indol-1-yl)cinnoline;
6,7-Dimethoxy-4-(1,3-thiazol-2-yl)cinnoline;
1-(6,7-Dimethoxy-1-naphthyl)-N-ethylindoline-5-sulfonamide;
1-(6,7-Dimethoxy-1-naphthyl)-N-isopropylindoline-5-sulfonamide;
N-cyclopropyl-1-(6,7-dimethoxycinnolin-4-yl)indoline-5-sulfonamide;
6,7-dimethoxy-4-[5-(pyrrolidin-1-ylsulfonyl)-2,3-dihydro-1H-indol-1-yl]cinnoline;
1-(6,7-dimethoxycinnolin-4-yl)-N,N-diisopropylindoline-5-sulfonamide;
1-(6,7-dimethoxycinnolin-4-yl)-N-(2-methoxyethyl)indoline-5-sulfonamide;
1-(6,7-dimethoxycinnolin-4-yl)-N-(2-morpholin-4-ylethyl)indoline-5-sulfonamide;
6,7-dimethoxy-4-(5-pyridin-4-yl-2,3-dihydro-1H-indol-1-yl)cinnoline;
6,7-dimethoxy-4-[7-(2-methoxyethoxy)-3,4-dihydroisoquinolin-2(1H)-yl]cinnoline;
4-[5-(3,5-dimethylisoxazol-4-yl)-2,3-dihydro-1H-indol-1-yl]-6,7-dimethoxycinnoline;
6,7-dimethoxy-4-(6-methoxy-2-naphthyl)cinnoline;
6,7-dimethoxy-4-[5-(piperidin-1-ylsulfonyl)-2,3-dihydro-1H-indol-1-yl]cinnoline;
6,7-dimethoxy-N-(5-methyl-4H-pyrazol-3-yl)cinnolin-4-amine;
6,7-dimethoxy-N-(4-methyl-1,3-thiazol-2-yl)cinnolin-4-amine;
3-(6,7-dimethoxycinnolin-4-yl)-N-ethylbenzamide;
3-(6,7-dimethoxycinnolin-4-yl)-N-isobutylbenzamide;
N-cyclopropyl-3-(6,7-dimethoxycinnolin-4-yl)benzamide;
6,7-bis(difluoromethoxy)-4-[7-(2-methoxyethoxy)-3,4-dihydroisoquinolin-2(1H)-yl]cinnoline;
2-(6,7-dimethoxycinnolin-4-yl)-6,7-dimethoxy-1,4-dihydroisoquinolin-3(2H)-one;
6-(benzyloxy)-2-(6,7-dimethoxycinnolin-4-yl)-3,4-dihydroisoquinolin-1(2H)-one;
2-(6,7-dimethoxycinnolin-4-yl)-5-hydroxy-3,4-dihydroisoquinolin-1(2H)-one;
6,7-dimethoxy-4-[6-(2-methoxyethoxy)-3,4-dihydroisoquinolin-2(1H)-yl]cinnoline;
6,7-bis(difluoromethoxy)-4-[7-(2-methoxyethoxy)-3,4-dihydroisoquinolin-2(1H)-yl]cinnoline;
N-cyclohexyl-3-(6,7-dimethoxycinnolin-4-yl)benzamide;
3-(6,7-dimethoxycinnolin-4-yl)-N,N-diethylbenzamide;
2-(6,7-dimethoxycinnolin-4-yl)-5-(2-methoxyethoxy)-3,4-dihydroisoquinolin-1(2H)-one;
4-(3,4-dihydronaphthalen-2-yl)-6,7-dimethoxycinnoline;
6,7-dimethoxy-4-[7-(tetrahydrofuran-3-yloxy)-3,4-dihydroisoquinolin-2(1H)-yl]cinnoline;
6,7-dimethoxy-4-[7-(2-morpholin-4-ylethoxy)-3,4-dihydroisoquinolin-2(1H)-yl]cinnoline;
2-{[2-(6,7-dimethoxycinnolin-4-yl)-1,2,3,4-tetrahydroisoquinolin-5-yl]oxy}ethanol;
4-[7-[2-(benzyloxy)ethoxy]-3,4-dihydroisoquinolin-2(1 H)-yl]-6,7-dimethoxycinnoline;
2-(6,7-dimethoxycinnolin-4-yl)-1,2,3,4-tetrahydroisoquinoline-5-carboxylic acid; or
6,7-dimethoxy-4-[8-(2-methoxyethoxy)-3,4-dihydroisoquinolin-2(1H)-yl]cinnoline; or a pharmaceutically acceptable salt thereof, or a solvate thereof, or a solvate of a pharmaceutically acceptable salt thereof, and
wherein if said compound exhibits chirality it can be in the form of a mixture of enantiomers, a mixture of diastereomers, or in the form of a single enantiomer or a single diastereomer.

155. The compound according to claim 98, wherein said compound is:
4-(6,7-Dimethoxy-3,4-dihydroisoquinolin-2(1H)-yl)-6,7-dimethoxycinnoline hydrochloride;
1-(6,7-Dimethoxy-1-naphthyl)-N-ethylindoline-5-sulfonamide hydroformate;
1-(6,7-Dimethoxy-1-naphthyl)-N-isopropylindoline-5-sulfonamide hydroformate;
6,7-dimethoxy-4-[7-(2-methoxyethoxy)-3,4-dihydroisoquinolin-2(1H)-yl]cinnoline hydrochloride;
6,7-dimethoxy-N-(5-methyl-4H-pyrazol-3-yl)cinnolin-4-amine hydroformate;
6,7-dimethoxy-N-(4-methyl-1,3-thiazol-2-yl)cinnolin-4-amine hydroformate;
6,7-bis(difluoromethoxy)-4-[7-(2-methoxyethoxy)-3,4-dihydroisoquinolin-2(1H)-yl]cinnoline hydroformate; or
4-(3,4-dihydronaphthalen-2-yl)-6,7-dimethoxycinnoline hydroformate;
or a solvate thereof; and
wherein if said compound exhibits chirality it can be in the form of a mixture of enantiomers, a mixture of diastereomers, or in the form of a single enantiomer or a single diastereomer.

156. The compound according to claim 98, wherein said compound is:
(4,5-Dihydro-4,4-dimethyl-1-isopropyl-1H-imidazol-2-yl) (6,7-dihydroxycinnolin-4-yl)-acetonitrile
(1-Benzyl-1H-imidazol-2-yl)(6,7-dimethoxycinnolin-4-yl)acetonitrile;
(6,7-Dimethoxycinnolin-4-yl)(pyridin-3-yl)acetonitrile;
(6,7-Dimethoxycinnolin-4-yl)[2-(trifluoromethyl)phenyl]acetonitrile;
(4,5-Dihydro-1-isopropyl-1H- imidazol-2-yl) (6,7-dimethoxycinnolin-4-yl)acetonitrile;
2-(6,7-Dimethoxycinnolin-4-yl)-1,2,3,4-tetrahydroisoquinoline-7-carbonitrile;
4-(3,4-Dihydro-6-methoxy-isoquinolin-2(1H)-yl)-6,7-dimethoxycinnoline;
4-(3,4-Dihydro-7-fluoro-isoquinolin-2(1H)-yl)-6,7-dimethoxycinnoline;
Methyl 2-(6,7-Dimethoxycinnolin-4-yl)-1,2,3,4-tetrahydroisoquinoline-5-carboxylate
4-(3,4-Dihydro-7-nitroisoquinolin-2(1H)-yl)-6,7-dimethoxycinnoline;
4-(6,7-Diethoxy-3,4-dihydro-isoquinolin-2(1H)-yl)-6,7-dimethoxycinnoline;
4-(3,4-Dihydro-5-nitroisoquinolin-2(1H)-yl)-6,7-dimethoxycinnoline;
4-(1,3-Dihydro-2H-isoindol-2-yl)-6,7-dimethoxycinnoline;
Methyl 2-(6,7-dimethoxycinnolin-4-yl)-1,2,3,4-tetrahydroisoquinoline-7-carboxylate;
4-[4-(3-CHlorophenyl)piperazin-1-yl]-6,7-dimethoxycinnoline;
4-(3,4-Dihydroisoquinolin-2(1H)-yl)-6,7-dimethoxycinnoline;
(6,7-Dimethoxy-cinnolin-4-yl)-(4,5-dihydro-(4S)-4-phenyl-oxazol-2-yl)acetonitrile;
4-(3,4-Dihydro-6,7-dimethoxy-isoquinolin-2(1H)-yl)-6,7-dimethoxycinnoline;
4-(3,4-Dihydroquinolin-1 (2H)-yl)-6,7-dimethoxycinnoline;
6,7-Dimethoxy-(4-morpholin-4-yl)cinnoline;
4-[4-(1,2-Benzisothiazol-3-yl)piperazin-1-yl]-6,7-dimethoxycinnoline;
6,7-Dimethoxy-4-[(4aR, 8aS)-octahydroisoquinolin-2(1H)-yl]cinnoline;
4-{4-[Bis(4-fluorophenyl)methyl]piperazin-1-yl }-6,7-dimethoxycinnoline;
6,7-Dimethoxy-4-piperidin-1-ylcinnoline;
4-[4-(1,3-Benzodioxol-5-ylmethyl)piperazin-1-yl]-6,7-dimethoxycinnoline;
6-(6,7-Dimethoxycinnolin-4-yl)-5,6,7,8-tetrahydro-[1,3]-dioxolo[4,5-g]isoquinoline; and
(1-Benzyl-4,5-dihydro-1H- imidazol-2-yl)(6,7-dimethoxycinnolin-4-yl)acetonitrile;
or a pharmaceutically acceptable salt thereof, or a solvate thereof, or a solvate of a pharmaceutically acceptable salt thereof, and
wherein if said compound exhibits chirality it can be in the form of a mixture of enantiomers, a mixture of diastereomers, or in the form of a single enantiomer or a single diastereomer.

157. A pharmaceutical composition comprising a compound according to claim 98 and a pharmaceutically acceptable carrier.

158. A method of inhibiting PDE1O enzyme in a patient in need thereof comprising administering to said patient an effective amount of a compound of Formula I or Formula II: 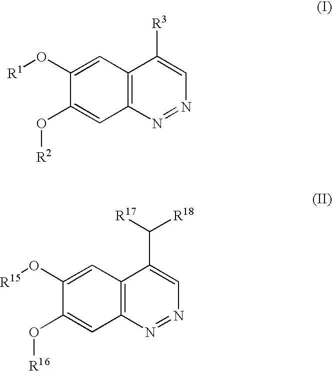
wherein:
R¹ is H or alkyl having 1 to 4 carbon atoms, which is unsubstituted or substituted one or more times by halogen;
R² is H or alkyl having 1 to 4 carbon atoms, which is unsubstituted or substituted one or more times by halogen;
R³ is selected from formulas (a)-(h): 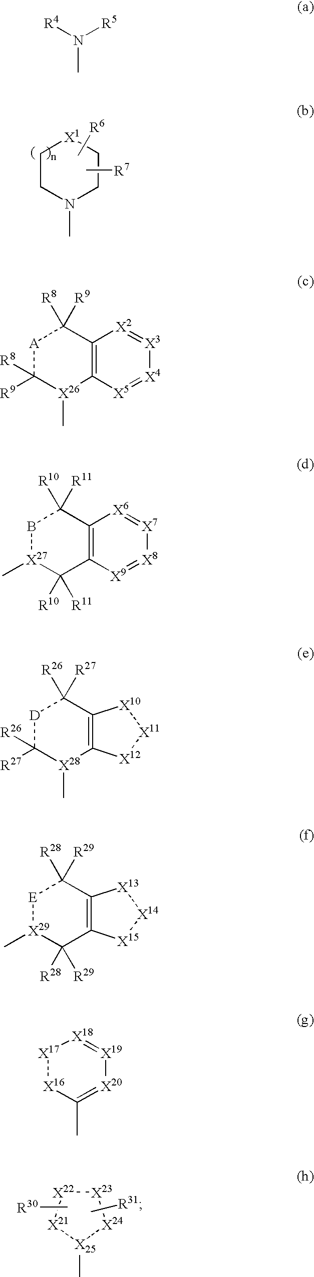
where:
n is 0, 1, 2, or 3;
-A- is a single bond, a double bond, —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁸—, ═CR⁸—CR⁸═, —CR⁸R⁹—CR⁸CR⁹—CR⁸CR⁹—, ═CR⁸—CR⁸CR⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, or ═CR —CR⁸—CR⁸R⁹—CR⁸═;
—B— is a single bond, a double bond, —CR¹⁰R¹¹—, ═CR¹⁰—, —CR¹⁰═, —CR¹⁰R¹¹—CR¹⁰R¹¹—, ═CR¹⁰—CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, ═CR¹⁰—CR¹⁰═CR¹⁰—, —CR¹⁰═CR¹⁰—CR¹⁰═, or ═CR¹⁰—CR¹⁰R¹¹-CR¹⁰═,
with the proviso that when X²⁷ is N, then —B— is not a double bond, ═CR¹⁰—, ═CR¹⁰—CR¹⁰R¹¹—CR¹⁰R¹¹—═CR¹⁰—CR¹⁰═, ═CR¹⁰—CR¹⁰R¹¹—CR¹⁰R¹¹—, ═CR¹⁰—CR¹⁰═CR¹⁰—, or ═CR¹⁰—CR¹⁰R¹¹—CR¹⁰═;
-D- is a single bond, a double bond, —CR²⁶R²⁷—, ═CR²⁶—, —CR²⁶═, —CR²⁶R²⁷—CR²⁶R²⁷—, ═CR²⁶—CR²⁶R²⁷—, —CR²⁶R²⁷—CR26═, —CR²⁶═CR²⁷—, ═CR²⁶—CR²⁶═, —CR²⁶R²⁷—CR²⁶R²⁷—CR²⁶R²⁷—, ═CR²⁶—CR²⁶R²⁷—CR²⁶CR²⁷—, —CR²⁶═CR²⁶—CR²⁶R²⁷—, —CR²⁶R²⁷—CR²═CR²⁶—, —CR²⁶R²⁷—CR²⁶R²⁷—CR²⁶═, ═CR²⁶—CR²⁶═CR²⁶—, —CR²⁶═CR²⁶—CR²⁶═, or ═CR²⁶—CR²⁶R²⁷—CR²⁶═;
-E- is a single bond, a double bond, —CR²⁸R²⁹R—, ═CR²⁹—, —CR²⁸═, —CR²⁸—, —CR²⁸═, —CR²⁸R²⁹—CR²⁸R²⁹—, ═CR²⁸—CR²⁸R²⁹—, —CR²⁸R²⁹—CR²⁸═, —CR²⁸═CR²⁹—, ═CR²⁹—, ═CR²⁸—CR²⁸═, —CR²⁸R²⁹—CR²⁸R²⁹—CR²⁸R²⁹—, ═CR²⁸—CR²⁸R²⁹—CR²⁸R²⁹—, —CR²⁸═CR²⁸—CR²⁸R²⁹—, —CR²⁸R²⁹—CR²⁸═CR²⁸—, —CR²⁸—, —CR²⁸R²⁹—CR²⁸R²⁹—CR²⁸═, ═CR²⁸—CR²⁹═CR²⁸—, —CR²⁸═CR²⁸—CR²⁸═, or ═CR²⁸—CR²⁸R²⁹—CR²⁸═,
with the proviso that when X²⁹ is N, then -E- is not a double bond, ═CR²⁸—, ═CR²⁸—CR²⁸R²²⁹—, ═CR²⁸—CR²⁸═, ═CR²⁸—CR²⁸R²⁹—CR²⁸R²⁹—, ═CR²⁸—CR²⁸═CR²⁸—CR²⁸—, or ═CR²⁸—CR²⁸R²⁹—CR²⁸═;
the dotted lines in forrnula (e) independently represent a single bond or a double bond, wherein there is at least one double bond between Xl and X¹¹ or X¹¹ and X¹²;
the dotted lines in forrnula (f) independently represent a single bond or a double bond, wherein there is at least one double bond between X¹³ and X¹⁴ or X¹⁴ and X¹⁵;
the dotted line in formnula (g) independently represents a single bond or a double bond;
the dotted lines in formula (h) independently represent a single bond or a double bond, with the proviso that when two double bonds are present, they are not adjacent to each other;
R⁴ and R⁵ are each independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR^{1 , C}₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof, arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₌₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
R⁶ and R⁷ are each independently:
H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹² NHSO₂R¹², —NR COR¹², —CONHR¹², —NHCONHR¹², —OCONHR¹², —NHCOOR¹², —SCONHR², —SCSNHR¹², or —NHCSNHR¹², or combinations thereof, or
R⁶ and R⁷ optionally form a cycloalkyl group, spiro or fused, having 3 to 8 carbon atoms, or
R⁶ and R⁷ together with the carbon to which they are attached form a C(═O) group;
X¹ is O, S, NR¹³,CH₂, CHR or CR⁶R⁷;
X², X³, X⁴, X⁵, X⁶, X⁷, X⁸, and X⁹ are each independently N or CR¹⁴, and wherein two adjacent X²-X⁹ groups can each be CR¹⁴ in which the two R¹⁴ groups are together a methylenedioxy, ethylenedioxy, difluoromethylenedioxy, or tetrafluoro-ethylenedioxy group, to form a fused ring structure;
X¹⁰, X¹¹, X¹², X¹³, X¹⁴, and X¹⁵are each independently S, O, N, NR¹⁴, C(R¹⁴)₂, or CR¹⁴;
X¹⁶, X¹⁷, X¹⁸, X¹⁹, and X²⁰, are each independently N or CR14,
X¹⁸ and X¹⁹ or X¹⁹ and X²⁰ optionally form a fused aryl or heteroaryl, each of which may be substituted by one or more R¹⁴ groups;
X²¹, X²², X²³, and X²⁴ are each independently O, S, N, NR¹⁴, CR¹⁴, or C(R¹⁴)₂;
X²⁵ is N, C or CR¹⁴;
wherein at least two of X²¹, X²², X²³, X²⁴, and X²⁵ are each, independently, O, S, N, or NR¹⁴;
X²⁶ is N or CR⁸;
X²⁷ is C, N, or CR¹⁰;
X²⁸ is N or CR²⁶
X²⁹ is C, N, or CR²⁸;
R⁸, R⁹, R¹⁰, R¹¹, R²⁶, R²⁷, R²⁸, and R²⁹ are, in each case, independently:
absent, H, or alkyl having 1 to 8 carbon atoms, cycloalkyl having 3 to 12 carbon atoms, or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or combinations thereof; or
R⁸ and R⁹, R¹⁰ and R¹¹, R²⁶, and R²⁷, and/or R²⁸ and R²⁹ together optionally-form a cycloalkyl group, spiro or fused, having 3 to 8 carbon atoms, or
one or more of R⁸ and R⁹ and the carbon atom to which they are attached, or one or more of R¹⁰ and R¹¹ and the carbon atom to which they are attached, or one or more of R²⁶ and R²⁷ and the carbon atom to which they are attached, or one or more of R²⁸ and R²⁹ and the carbon atom to which they are attached, in each case form a C(═O) group;
R¹² is H or alkyl having 1 to 8 carbon atoms, cycloalkyl having 3 to 12 carbon atoms, or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or combinations thereof;
R¹³ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄- alkylsulphonyl, —SO₂NHR¹², —NHSO₂R¹², —NR¹²COR¹², —CONHR¹², —NHCONHR¹², —OCONHR¹², —NHCOOR¹², —SCONHR¹², —SCSNHR¹², or —NHCSNHR¹² or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—,
aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
—CH(aryl)₂ wherein each aryl group has 6 to 14 carbon atoms and is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₁₋₄ alkyl, halogenated C₁₋₄ alkyl, hydroxy, C₁₋₄-alkoxy, halogenated C, ₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heteroarylalkyl wherein the heteroaryl portion has 5 to 10 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, heterocycle, which is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxy-carbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁴ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCPR¹², C₁₋₄-alkylthio, C₁₋₄-alkylsulphinyl, C₁₋₄-alkylsulphonyl, —SO₂NHR¹², —NHSO₂R¹², —NR COR¹², —CONHR¹², —NHCONHR¹², —OCONHR¹², —NHCOOR¹², —SCONHR¹², —SCSNHR¹², or —NHCSNHR or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—,
a heterocyclic group, which is saturated, partially saturated, or unsaturated, having 5 to 10 ring atoms in which at least 1 ring atom is a heteroatom which is unsubstituted or substituted one or more times by halogen, C₆₋₁₄ aryl, arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
arylalkyl having 7 to 16 carbon atoms which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroarylalkyl wherein the heteroaryl portion has 5 to 1 0 ring atoms in which at least 1 ring atom is a heteroatom and the alkyl portion has 1 to 3 carbon atoms, the heteroaryl portion is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
aryloxy having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryloxy having 5 to 10 ring atoms in which at least 1 ring atom is a heteroatom which is unsubstituted or substituted one or more times by halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
O-heterocyclic group, in which the heterocyclic group is nonaromatic, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
O-heterocyclicalkyl group, in which the heterocyclic group is nonaromatic, having 5 to 10 ring atoms in which at least 1 ring atom is a heteroatom, and the alkyl portion has 1 to 3 carbon atoms and the heterocyclic group is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof; or
halogen, hydroxy, C₁₋₄-alkoxy, C₁₋₄-alkyloxyC₁₋₄-alkoxy, C₄₋₂-cycloalkylalkyloxy, C₁₋₄-alkyloxyC₇₋₁₆-arylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹², —COOR¹², —OCOR¹², C₁₋₄-alkylthio, C₁₋₄-alkylsulphinyl, C₁₋₄-alkylsulphonyl, —SO₂NHR¹², —SO₂NHR²⁵, —SO₂NR¹⁹R²⁵, —SO₂R³², —NHSO₂R¹², —NR¹²COR¹², —CONHR¹², NHCONHR¹², —OCONHR¹², —NHCOOR¹², —SCONHR¹², —SCSNHR¹², or —NHCSNHR¹²;
R¹⁵ is H or alkyl having 1 to 4 carbon atoms, which is unsubstituted or substituted one or more times by halogen;
R¹⁶ is H or alkyl having 1 to 4 carbon atoms, which is unsubstituted or substituted one or more times by halogen;
R¹⁷ is aryl having 6 to 14 carbon atoms, which is unsubstituted or substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof,
heteroaryl having 5 to 10 ring atoms in which at least 1 ring atom is a heteroatom which is unsubstituted or substituted one or more times by halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof,
heterocycle, which is nonaromatic, having 5 to 1 0 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
carbocycle which is a nonaromatic, monocyclic or bicyclic, group having 5 to 14 carbon atoms, which is unsubstituted or is substituted one or more times by halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, phenoxy, or combinations thereof;
R¹⁸ is H,
straight, branched or cyclic alkyl having up to 12 carbon atoms which is unsubstituted or substituted one or more times by halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR¹⁹, —NHSO₂R¹⁹, —NR¹⁹COR¹⁹, —CONHR¹⁹, —NHCONHR¹⁹, —OCONHR 9, —NHCOOR¹⁹, —SCONHR19, —SCSNHR¹⁹, or —NHCSNHR¹⁹ or combinations thereof, wherein optionally one or more —CH₂— groups is, in each case independently, replaced by —O—, —S—, or —NH—, and wherein optionally one or more —CH₂CH₂— groups is replaced in each case by —CH═CH— or —C—C—, or
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁹, —COOR¹⁹, —OCOR¹⁹, C₁₋₄-alkylthio, C₁₋₄-alkylsulphinyl, C₁₋₄-alkylsulphonyl, —SO₂NHR19, —NHSO₂R¹⁹, —NR¹⁹COR¹⁹, —CONHR¹⁹, —NHCONHR¹⁹, —OCONHR¹⁹, —NHCOOR¹⁹, —SCONHR¹⁹, —SCSNHR¹⁹, or —NHCSNHR19, or combinations thereof;
R¹⁹ is H or alkyl having 1 to 8 carbon atoms, which is branched or unbranched and which is unsubstituted or substituted one or more times with halogen, C₁₋₄-alkyl, C₁₋₄-alkoxy, oxo, or combinations thereof;
R²⁵ is H,
alkyl having 1 to 8 carbon atoms, which is branched or unbranched and which is unsubstituted or substituted one or more times with halogen, C₁₋₄-alkyl, C₁₋₄-alkoxy, oxo, or combinations thereof;
cycloalkyl having 3 to 10 carbon atoms, which is unsubstituted or substituted one or more times with halogen, C₁₋₄-alkyl, C₁₋₄-alkoxy, oxo, or combinations thereof, cycloalkylalkyl having 4-12 carbon atoms which is unsubstituted or substituted one or more times with halogen, C₁₋₄-alkyl, C₁₋₄-alkoxy, oxo, or combinations thereof,
heterocyclic group which is saturated, partially saturated, or unsaturated, having 5 to 10 ring atoms in which at least 1 ring atom is a heteroatom, and is unsubstituted or is substituted one or more times by halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof, or
heterocyclicalkyl group wherein the heterocyclic group has 5 to 10 ring atoms in which at least 1 ring atom is a heteroatom and the alkyl portion has 1 to 4 carbon atoms, the heterocyclic group is unsubstituted or is substituted one or more times by halogen C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof;
R³⁰ and R³¹ are, in each case, independently:
H or alkyl having 1 to 8 carbon atoms, cycloalkyl having 3 to 12 carbon atoms or cycloalkylalkyl having 4 to 12 carbon atoms, each of which is branched or unbranched and which is unsubstituted or substituted one or more times with halogen, C₁₋₄-alkyl, C₁₋₄-alkoxy, oxo, or combinations thereof; or
R³⁰ and R³¹ form a cycloalkyl group, spiro or fused, having 3 to 8 carbon atoms, or R³⁰ and R³¹ and the carbon atom to which they are attached form a C(═O) group; and
R³² is a heterocyclic group which is saturated or partially saturated and has 5 to 10 ring atoms in which at least 1 ring atom is a heteroatom and which is unsubstituted or substituted one or more times by halogen, C₆-₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, C₁₋₄-alkylsulphonyl, or combinations thereof; or
a solvate, a single enantiomer, a single diastereomer, a mixture of enantiomers, a mixture of diastereomers, a polymorph, or a pharmaceutically acceptable salt thereof.

159. The method of claim 158 wherein the patient is suffering from a psychiatric or neurological syndrome.

160. The method of claim 158 wherein the patient is suffering from bipolar disorder, schizophrenia, obsessive-compulsive disorder, Parkinson's disease, Alzheimer's disease, multiple sclerosis, Huntington's disease, a disorder affecting the function of the basal ganglia, diabetes or obesity.
